(19) United States
(12) Patent Application Publication
(10) Pub. No.: US 2018/0298009 A1
(43) Pub. Date: Oct. 18, 2018

(54) Fused Pyrazole Derivatives As Kinase Inhibitors

(71) Applicants: UCB Biopharma SPRL, Brussels (BE); Katholieke Universiteit Leuven, K.U.Leuven R&D, Leuven (BE)

(72) Inventors: Daniel James Ford, Slough, Berkshire (GB); Helen Tracey Horsley, Slough, Berkshire (GB); James Thomas Reuberson, Slough, Berkshire (GB)

(21) Appl. No.: 15/762,670

(22) PCT Filed: Sep. 28, 2016
(86) PCT No.: PCT/EP2016/073028

§ 371 Date: Mar. 23, 2018

(30) Foreign Application Priority Data
Sep. 30, 2015 (GB) 1517263.8

(51) Int. Cl. C07D 487/04; C07D 498/04

(52) U.S. Cl. CPC C07D 487/04; C07D 498/04

(57) ABSTRACT

A series of substituted pyrazolo[1,5-a]pyrimidine and pyrazolo[1,5-a][1,3,5]-triazine derivatives of formula (I), as defined herein, being selective inhibitors of phosphatidylinositol-4-kinase IIIβ (PI4KIIIβ) activity, are beneficial in the treatment and/or prevention of various human ailments, including inflammatory, autoimmune and oncological disorders; viral diseases and malaria; and organ and cell transplant rejection.

The present invention relates to a class of fused pyrazole derivatives, and to their use in therapy. More particularly, the present invention provides substituted pyrazolo[1,5-a]pyrimidine and pyrazolo[1,5-a][1,3,5]triazine derivatives. These compounds are selective inhibitors of phosphatidylinositol-4-kinase IIIβ (PI4KIIIβ) activity, and are accordingly of benefit as pharmaceutical agents, especially in the treatment of adverse inflammatory, autoimmune and oncological disorders, in the treatment of viral diseases and malaria, and in the management of organ and cell transplant rejection.

In addition, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/034738 discloses that inhibitors of PI4KIIIβ activity are useful as medicaments for the treatment of autoimmune and inflammatory disorders, and organ and cell transplant rejection.

Inhibitors of PI4KIIIβ have been identified as molecules with an ideal activity profile for the prevention, treatment and elimination of malaria (cf. C. W. McNamara et al., Nature, 2013, 504, 248-253).

WO 2010/103130 describes a family of oxazolo[5,4-d]pyrimidine, thiazolo[5,4-d]-pyrimidine, thieno[2,3-d]pyrimidine and purine derivatives that are active in a range of assays, including the Mixed Lymphocyte Reaction (MLR) test, and are stated to be effective for the treatment of immune and autoimmune disorders, and organ and cell transplant rejection. WO 2011/147753 discloses the same family of compounds as having significant antiviral activity. Furthermore, WO 2012/035423 discloses the same family of compounds as having significant anticancer activity.

WO 2013/024291, WO 2013/068458, WO 2014/053581 and WO 2014/096423 describe various series of fused pyrimidine derivatives that are stated to be of benefit as pharmaceutical agents, especially in the treatment of adverse inflammatory, autoimmune and oncological disorders, in the treatment of viral diseases, and in the management of organ and cell transplant rejection.

Copending international patent applications PCT/EP2015/063048, PCT/EP2015/063051 and PCT/EP2015/063052 (published on 23 Dec. 2015 as WO 2015/193167, WO 2015/193168 and WO 2015/193169 respectively) describe various series of fused bicyclic heteroaromatic derivatives that are stated to be selective inhibitors of PI4KIIIβ activity, and accordingly of benefit as pharmaceutical agents, especially in the treatment of adverse inflammatory, autoimmune and oncological disorders, in the treatment of viral diseases, and in the management of organ and cell transplant rejection.

Various classes of substituted fused bicyclic heteroaromatic compounds that are stated to be selective PI4KIIIβ inhibitors, and to exhibit antiviral activity, are described in the scientific literature (cf. I. Mejdroveá et al., J. Med. Chem., 2015, 58, 3767-3793; A. M. MacLeod et al., ACS Med. Chem. Lett., 2013, 4, 585-589; and M. Arita et al., J. Virol., 2011, 85, 2364-2372).

None of the prior art available to date, however, discloses or suggests the precise structural class of fused pyrazole derivatives as provided by the present invention as having activity as PI4KIIIβ inhibitors.

The compounds of the present invention are potent and selective inhibitors of PI4KIIIβ activity, inhibiting the kinase affinity of human PI4KIIIβ (IC₅₀) at concentrations of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound). The compounds of the invention may possess at least a 10-fold selective affinity, typically at least a 20-fold selective affinity, suitably at least a 50-fold selective affinity, and ideally at least a 100-fold selective affinity, for human PI4KIIIβ relative to other human kinases.

Certain compounds in accordance with the present invention are active as inhibitors when subjected to the Mixed Lymphocyte Reaction (MLR) test. The MLR test is predictive of immunosuppression or immunomodulation. Thus, when subjected to the MLR test, certain compounds of the present invention display an IC₅₀ value of 10 μM or less, generally of 5 μM or less, usually of 2 μM or less, typically of 1 μM or less, suitably of 500 nM or less, ideally of 100 nM or less, and preferably of 20 nM or less (again, the skilled person will appreciate that a lower IC₅₀ figure denotes a more active compound).

The present invention provides a compound of formula (I) or an N-oxide thereof, or a pharmaceutically acceptable salt or solvate thereof:

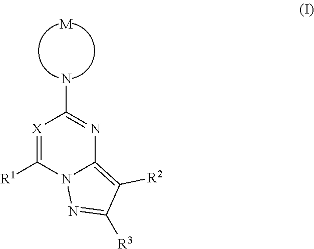

wherein

X represents N or CH;

M represents the residue of an optionally substituted saturated four-, five-, six- or seven-membered monocyclic ring containing one nitrogen atom and 0, 1, 2 or 3 additional heteroatoms independently selected from N, O and S, but containing no more than one O or S atom; or

M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom; or

M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom; or

M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom;

R¹ and R² independently represent hydrogen, halogen, cyano, nitro, hydroxy, trifluoromethyl, trifluoromethoxy, —OR^{a}, —SR^{a}, —SOR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b} or —SO₂NR^{b}R^{c}; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, heteroaryl or heteroaryl(C₁₋₆)alkyl, any of which groups may be optionally substituted by one or more substituents;

R³ represents hydrogen, halogen, cyano, trifluoromethyl or C₁₋₆ alkyl;

R^{a} represents hydrogen; or R^{a} represents C₁₋₆ alkyl, aryl, aryl(C₁₋₆)alkyl, heteroaryl or heteroaryl(C₁₋₆)alkyl, any of which groups may be optionally substituted by one or more substituents;

R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or

R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;

R^{d} represents hydrogen; or C₁₋₆ alkyl, C₃₋₇ cycloalkyl, aryl, C₃₋₇ heterocycloalkyl or heteroaryl, any of which groups may be optionally substituted by one or more substituents; and

R^{e} represents C₁₋₆ alkyl, aryl or heteroaryl, any of which groups may be optionally substituted by one or more substituents.

Where any of the groups in the compounds of formula (I) above is stated to be optionally substituted, this group may be unsubstituted, or substituted by one or more substituents. Typically, such groups will be unsubstituted, or substituted by one or two substituents.

For use in medicine, the salts of the compounds of formula (I) will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulfuric acid, methanesulfonic acid, fumaric acid, maleic acid, succinic acid, acetic acid, benzoic acid, citric acid, tartaric acid or phosphoric acid. Furthermore, where the compounds of the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope solvates of the compounds of formula (I) above. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

Suitable alkyl groups which may be present on the compounds of the invention include straight-chained and branched C₁₋₆ alkyl groups, for example C₁₋₄ alkyl groups. Typical examples include methyl and ethyl groups, and straight-chained or branched propyl, butyl, pentyl and hexyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulfonyl” and “C₁₋₆ alkylamino” are to be construed accordingly.

Suitable C₂₋₆ alkenyl groups include vinyl, allyl and prop-1-en-2-yl.

Suitable C₃₋₇ cycloalkyl groups, which may comprise benzo-fused analogues thereof, include cyclopropyl, cyclobutyl, cyclopentyl, indanyl, cyclohexyl and cycloheptyl.

Suitable aryl groups include phenyl and naphthyl, preferably phenyl.

Suitable aryl(C₁₋₆)alkyl groups include benzyl, phenylethyl, phenylpropyl and naphthylmethyl.

Suitable heterocycloalkyl groups, which may comprise benzo-fused analogues thereof, include oxetanyl, azetidinyl, tetrahydrofuranyl, dihydrobenzofuranyl, dihydroisobenzofuranyl, pyrrolidinyl, indolinyl, thiazolidinyl, imidazolidinyl, tetrahydropyranyl, chromanyl, piperidinyl, 1,2,3,4-tetrahydroquinolinyl, 1,2,3,4-tetrahydroisoquinolinyl, piperazinyl, 1,2,3,4-tetrahydroquinoxalinyl, homopiperazinyl, morpholinyl, benzoxazinyl and thiomorpholinyl.

Examples of suitable heterocycloalkenyl groups include oxazolinyl.

Suitable heteroaryl groups include furyl, benzofuryl, dibenzofuryl, thienyl, benzothienyl, dibenzothienyl, pyrrolyl, indolyl, pyrrolo[2,3-b]pyridinyl, pyrrolo[3,2-d-pyridinyl, pyrazolyl, pyrazolo[1,5-a]pyridinyl, pyrazolo[3,4-d]pyrimidinyl, indazolyl, oxazolyl, benzoxazolyl, isoxazolyl, thiazolyl, benzothiazolyl, isothiazolyl, imidazolyl, imidazo[2,1-b]thiazolyl, benzimidazolyl, imidazo[1,2-a]pyridinyl, imidazo[1,5-a]-pyridinyl, imidazo[4,5-b]pyridinyl, purinyl, imidazo[1,2-a]pyrimidinyl, imidazo[1,2-a]-pyrazinyl, oxadiazolyl, benzoxadiazolyl, thiadiazolyl, benzothiadiazolyl, triazolyl, benzotriazolyl, [1,2,4]triazolo[4,3-a]pyridinyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds of the invention possess two or more asymmetric centres, they may additionally exist as diastereomers. The invention is to be understood to extend to all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In one embodiment, X represents N. In another embodiment, X represents CH.

Individual sub-classes of compounds in accordance with the present invention are represented by the compounds of formula (IA) and (IB):

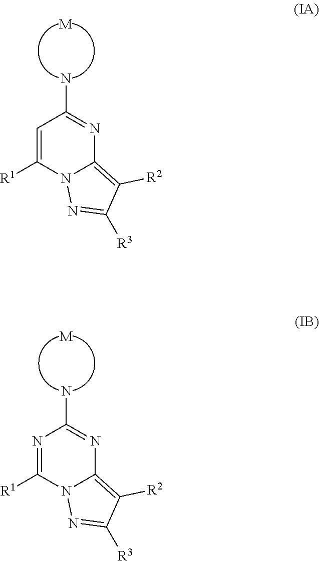

wherein M, R¹, R² and R³ are as defined above.

In a first aspect, M represents the residue of an optionally substituted saturated four-, five-, six- or seven-membered monocyclic ring containing one nitrogen atom and 0, 1, 2 or 3 additional heteroatoms independently selected from N, O and S, but containing no more than one O or S atom.

In a first embodiment, M represents the residue of an optionally substituted saturated four-membered monocyclic ring. In a second embodiment, M represents the residue of an optionally substituted saturated five-membered monocyclic ring. In a third embodiment, M represents the residue of an optionally substituted saturated six-membered monocyclic ring. In a fourth embodiment, M represents the residue of an optionally substituted saturated seven-membered monocyclic ring.

In a first embodiment, the monocyclic ring of which M is the residue contains one nitrogen atom and no additional heteroatoms (i.e. it is an optionally substituted azetidin-1-yl, pyrrolidin-1-yl, piperidin-1-yl or azepan-1-yl ring). In a second embodiment, the monocyclic ring of which M is the residue contains one nitrogen atom and one additional heteroatom selected from N, O and S. In a third embodiment, the monocyclic ring of which M is the residue contains one nitrogen atom and two additional heteroatoms selected from N, O and S, of which not more than one is O or S. In a fourth embodiment, the monocyclic ring of which M is the residue contains one nitrogen atom and three additional heteroatoms selected from N, O and S, of which not more than one is O or S.

Typical values of the monocyclic ring of which M is the residue include azetidin-1-yl, pyrrolidin-1-yl, imidazolidin-1-yl, piperidin-1-yl, morpholin-4-yl, thiomorpholin-4-yl, piperazin-1-yl, azepan-1-yl and [1,4]diazepan-1-yl, any of which rings may be optionally substituted by one or more substituents.

Selected values of the monocyclic ring of which M is the residue include azetidin-1-yl, pyrrolidin-1-yl, piperidin-1-yl, morpholin-4-yl, piperazin-1-yl, azepan-1-yl and [1,4]diazepan-1-yl, any of which rings may be optionally substituted by one or more substituents.

Suitable values of the monocyclic ring of which M is the residue include azetidin-1-yl, morpholin-4-yl, piperazin-1-yl and azepan-1-yl, any of which rings may be optionally substituted by one or more substituents.

A particular value of the monocyclic ring of which M is the residue is optionally substituted piperazin-1-yl.

In a second aspect, M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom.

In a first embodiment, M represents the residue of an optionally substituted saturated or unsaturated five-membered fused bicyclic ring system. In a second embodiment, M represents the residue of an optionally substituted saturated or unsaturated six-membered fused bicyclic ring system. In a third embodiment, M represents the residue of an optionally substituted saturated or unsaturated seven-membered fused bicyclic ring system. In a fourth embodiment, M represents the residue of an optionally substituted saturated or unsaturated eight-membered fused bicyclic ring system. In a fifth embodiment, M represents the residue of an optionally substituted saturated or unsaturated nine-membered fused bicyclic ring system. In a sixth embodiment, M represents the residue of an optionally substituted saturated or unsaturated ten-membered fused bicyclic ring system.

In a first embodiment, the fused bicyclic ring system of which M is the residue is saturated. In a second embodiment, the fused bicyclic ring system of which M is the residue is unsaturated.

In a first embodiment, the fused bicyclic ring system of which M is the residue contains one nitrogen atom and no additional heteroatoms. In a second embodiment, the fused bicyclic ring system of which M is the residue contains one nitrogen atom and one additional heteroatom selected from N, O and S. In a third embodiment, the fused bicyclic ring system of which M is the residue contains one nitrogen atom and two additional heteroatoms selected from N, O and S, of which not more than one is O or S. In a fourth embodiment, the fused bicyclic ring system of which M is the residue contains one nitrogen atom and three additional heteroatoms selected from N, O and S, of which not more than one is O or S.

Illustrative values of the fused bicyclic ring system of which M is the residue include 1,2,3,3a,4,5,6,6a-octahydrocyclopenta[c]pyrrol-2-yl, 2,3,4,4a,5,6,7,7a-octahydro-pyrrolo[3,4-b][1,4]oxazin-6-yl, 1,2,3,4,6,7,8,8a-octahydropyrrolo[1,2-a]pyrazin-2-yl and 4,5,6,7-tetrahydropyrazolo[1,5-a]pyrazin-5-yl, any of which ring systems may be optionally substituted by one or more substituents.

Selected values of the fused bicyclic ring system of which M is the residue include 1,2,3,3a,4,5,6,6a-octahydrocyclopenta[c]pyrrol-2-yl, 2,3,4,4a,5,6,7,7a-octahydropyrrolo-[3,4-b][1,4]oxazin-6-yl and 1,2,3,4,6,7,8,8a-octahydropyrrolo[1,2-a]pyrazin-2-yl, any of which ring systems may be optionally substituted by one or more substituents.

Typical values of the fused bicyclic ring system of which M is the residue include 1,2,3,4,6,7,8,8a-octahydropyrrolo[1,2-a]pyrazin-2-yl and 4,5,6,7-tetrahydropyrazolo[1,5-a]pyrazin-5-yl, either of which ring systems may be optionally substituted by one or more substituents.

Suitable values of the fused bicyclic ring system of which M is the residue include 1,2,3,4,6,7,8,8a-octahydropyrrolo[1,2-a]pyrazin-2-yl, which ring system may be optionally substituted by one or more substituents.

In a third aspect, M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom.

In a first embodiment, M represents the residue of an optionally substituted saturated five-membered bridged bicyclic ring system. In a second embodiment, M represents the residue of an optionally substituted saturated six-membered bridged bicyclic ring system. In a third embodiment, M represents the residue of an optionally substituted saturated seven-membered bridged bicyclic ring system. In a fourth embodiment, M represents the residue of an optionally substituted saturated eight-membered bridged bicyclic ring system. In a fifth embodiment, M represents the residue of an optionally substituted saturated nine-membered bridged bicyclic ring system.

In a first embodiment, the bridged bicyclic ring system of which M is the residue contains one nitrogen atom and no additional heteroatoms. In a second embodiment, the bridged bicyclic ring system of which M is the residue contains one nitrogen atom and one additional heteroatom selected from N, O and S. In a third embodiment, the bridged bicyclic ring system of which M is the residue contains one nitrogen atom and two additional heteroatoms selected from N, O and S, of which not more than one is O or S. In a fourth embodiment, the bridged bicyclic ring system of which M is the residue contains one nitrogen atom and three additional heteroatoms selected from N, O and S, of which not more than one is O or S.

Typical values of the bridged bicyclic ring system of which M is the residue include 3-azabicyclo[3.1.0]hexan-3-yl, 2-oxa-5-azabicyclo[2.2.1]heptan-5-yl, 6-azabicyclo[3.2.0]heptan-6-yl, 3-azabicyclo[3.1.1]heptan-3-yl, 3-azabicyclo[4.1.0]heptan-3-yl, 2-oxa-5-azabicyclo[2.2.2]octan-5-yl, 3-azabicyclo[3.2.1]octan-3-yl, 8-azabicyclo-[3.2.1]octan-8-yl, 3-oxa-8-azabicyclo[3.2.1]octan-8-yl, 3,8-diazabicyclo[3.2.1]octan-3-yl, 3,8-diazabicyclo[3.2.1]octan-8-yl, 3,6-diazabicyclo[3.2.2]nonan-3-yl, 3,6-diazabicyclo-[3.2.2]nonan-6-yl, 3-oxa-7-azabicyclo[3.3.1]nonan-7-yl, 3,9-diazabicyclo[4.2.1]nonan-3-yl and 3,9-diazabicyclo[4.2.1]nonan-9-yl, any of which ring systems may be optionally substituted by one or more substituents.

Selected values of the bridged bicyclic ring system of which M is the residue include 3-azabicyclo[3.1.0]hexan-3-yl, 2-oxa-5-azabicyclo[2.2.1]heptan-5-yl and 8-azabicyclo[3.2.1]octan-8-yl, any of which ring systems may be optionally substituted by one or more substituents.

In a fourth aspect, M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom.

In a first embodiment, M represents the residue of an optionally substituted saturated five-membered spirocyclic ring system. In a second embodiment, M represents the residue of an optionally substituted saturated six-membered spirocyclic ring system. In a third embodiment, M represents the residue of an optionally substituted saturated seven-membered spirocyclic ring system. In a fourth embodiment, M represents the residue of an optionally substituted saturated eight-membered spirocyclic ring system. In a fifth embodiment, M represents the residue of an optionally substituted saturated nine-membered spirocyclic ring system.

In a first embodiment, the spirocyclic ring system of which M is the residue contains one nitrogen atom and no additional heteroatoms. In a second embodiment, the spirocyclic ring system of which M is the residue contains one nitrogen atom and one additional heteroatom selected from N, O and S. In a third embodiment, the spirocyclic ring system of which M is the residue contains one nitrogen atom and two additional heteroatoms selected from N, O and S, of which not more than one is O or S. In a fourth embodiment, the spirocyclic ring system of which M is the residue contains one nitrogen atom and three additional heteroatoms selected from N, O and S, of which not more than one is O or S.

Typical values of the spirocyclic ring system of which M is the residue include 5-azaspiro[2.3]hexan-5-yl, 5-azaspiro[2.4]heptan-5-yl, 2-azaspiro[3.3]heptan-2-yl, 2-oxa-6-azaspiro[3.3]heptan-6-yl, 2-oxa-6-azaspiro[3.4]octan-6-yl, 2-oxa-6-azaspiro[3.5]nonan-2-yl, 7-oxa-2-azaspiro[3.5]nonan-2-yl and 2-oxa-7-azaspiro[3.5]nonan-7-yl, any of which ring systems may be optionally substituted by one or more substituents.

Suitable values of the spirocyclic ring system of which M is the residue include 2-oxa-6-azaspiro[3.3]heptan-6-yl, which ring system may be optionally substituted by one or more substituents.

Appositely, M represents the residue of an azetidin-1-yl, pyrrolidin-1-yl, piperidin-1-yl, morpholin-4-yl, piperazin-1-yl, azepan-1-yl or [1,4]diazepan-1-yl ring, any of which rings may be optionally substituted by one or more substituents; or M represents the residue of a 1,2,3,3a,4,5,6,6a-octahydrocyclopenta[c]pyrrol-2-yl, 2,3,4,4a,5,6,7,7a-octahydropyrrolo[3,4-b][1,4]oxazin-6-yl, 1,2,3,4,6,7,8,8a-octahydropyrrolo[1,2-a]-pyrazin-2-yl, 3-azabicyclo[3.1.0]hexan-3-yl, 2-oxa-5-azabicyclo[2.2.1]heptan-5-yl, 8-azabicyclo[3.2.1]octan-8-yl or 2-oxa-6-azaspiro[3.3]heptan-6-yl ring system, any of which ring systems may be optionally substituted by one or more substituents.

Suitably, M represents the residue of an azetidin-1-yl, morpholin-4-yl, piperazin-1-yl or azepan-1-yl ring, any of which rings may be optionally substituted by one or more substituents; or M represents the residue of a 1,2,3,4,6,7,8,8a-octahydropyrrolo[1,2-a]-pyrazin-2-yl or 2-oxa-6-azaspiro[3.3]heptan-6-yl ring system, either of which ring systems may be optionally substituted by one or more substituents.

In a first embodiment, the cyclic moiety of which M is the residue is unsubstituted. In a second embodiment, the cyclic moiety of which M is the residue is substituted by one or more substituents. In one subset of that embodiment, the cyclic moiety of which M is the residue is monosubstituted. In another subset of that embodiment, the cyclic moiety of which M is the residue is disubstituted.

Typical examples of optional substituents on the cyclic moiety of which M is the residue include one, two or three substituents independently selected from halogen, C₁₋₆ alkyl, benzyl, heteroaryl, C₁₋₆ alkoxy, difluoromethoxy, trifluoromethoxy, C₁₋₆ alkoxy-(C₁₋₆)alkyl, C₁₋₆ alkylthio, C₁₋₆ alkylsulfonyl, hydroxy, hydroxy(C₁₋₆)alkyl, cyano, trifluoromethyl, oxo, C₂₋₆ alkylcarbonyl, hydroxy(C₁₋₆)alkylcarbonyl, di(C₁₋₆)alkylamino-(C₁₋₆)alkylcarbonyl, carboxy, carboxy(C₁₋₆)alkyl, C₂₋₆ alkoxycarbonyl, C₂₋₆ alkoxy-carbonyl(C₁₋₆)alkyl, amino, amino(C₁₋₆)alkyl, C₁₋₆ alkylamino, di(C₁₋₆)alkylamino, phenylamino, pyridinylamino, C₂₋₆ alkylcarbonylamino, hydroxy(C₁₋₆)alkylcarbonyl-amino, (C₃₋₇)cycloalkylcarbonylamino, C₂₋₆ alkoxycarbonylamino, C₁₋₆ alkylsulfonyl-amino, aminocarbonyl, C₁₋₆ alkylaminocarbonyl, di(C₁₋₆)alkylaminocarbonyl, aminocarbonyl(C₁₋₆)alkyl, (C₁₋₆)alkylaminocarbonyl(C₁₋₆)alkyl, di(C₁₋₆)alkylamino-carbonyl(C₁₋₆)alkyl and (C₁₋₆ alkoxy)(C₁₋₆ alkyl)phenylaminocarbonyl. Additional examples include (C₁₋₆)alkylheteroaryl, di(C₁₋₆)alkylamino(C₁₋₆)alkyl, N—[(C₁₋₆)alkyl]-N—[(C₂₋₆)alkylcarbonyl]amino, C₃₋₆ alkenyloxycarbonylamino, morpholinyl, dioxo-thiomorpholinyl, morpholinylcarbonyl and pyrrolidinylcarbonyl(C₁₋₆)alkyl.

Selected examples of optional substituents on the cyclic moiety of which M is the residue include one, two or three substituents independently selected from halogen, C₁₋₆ alkyl, benzyl, heteroaryl, (C₁₋₆)alkylheteroaryl, C₁₋₆ alkoxy, C₁₋₆ alkoxy(C₁₋₆)alkyl, C₁₋₆ alkylsulfonyl, oxo, C₂₋₆ alkylcarbonyl, C₂₋₆ alkoxycarbonyl, di(C₁₋₆)alkylamino, di(C₁₋₆)-alkylamino(C₁₋₆)alkyl, morpholinyl, dioxothiomorpholinyl, N—[(C₁₋₆)alkyl]-N—[(C₂₋₆)alkyl-carbonyl]amino, C₂₋₆ alkoxycarbonylamino, C₃₋₆ alkenyloxycarbonylamino, aminocarbonyl, di(C₁₋₆)alkylaminocarbonyl, (C₁₋₆ alkoxy)(C₁₋₆ alkyl)phenylamino-carbonyl, morpholinylcarbonyl and pyrrolidinylcarbonyl(C₁₋₆)alkyl.

Suitable examples of optional substituents on the cyclic moiety of which M is the residue include one, two or three substituents independently selected from halogen, C₁₋₆ alkyl, benzyl, heteroaryl, oxo, C₂₋₆ alkylcarbonyl, C₂₋₆ alkoxycarbonyl and (C₁₋₆ alkoxy)-(C₁₋₆ alkyl)phenylaminocarbonyl.

Typical examples of specific substituents on the cyclic moiety of which M is the residue include one, two or three substituents independently selected from fluoro, chloro, bromo, methyl, ethyl, propyl, isopropyl, benzyl, pyridinyl, pyrazinyl, methoxy, isopropoxy, difluoromethoxy, trifluoromethoxy, methoxymethyl, methylthio, ethylthio, methylsulfonyl, hydroxy, hydroxymethyl, hydroxyethyl, cyano, trifluoromethyl, oxo, acetyl, ethylcarbonyl, tert-butylcarbonyl, hydroxyacetyl, dimethylaminoacetyl, carboxy, carboxymethyl, methoxycarbonyl, ethoxycarbonyl, tert-butoxycarbonyl, methoxy-carbonylmethyl, ethoxycarbonylmethyl, amino, aminomethyl, methylamino, ethylamino, dimethylamino, phenylamino, pyridinylamino, acetylamino, hydroxyacetylamino, cyclopropylcarbonylamino, tert-butoxycarbonylamino, methylsulfonylamino, amino-carbonyl, methylaminocarbonyl, dimethylaminocarbonyl, aminocarbonylmethyl, methylaminocarbonylmethyl, dimethylaminocarbonylmethyl and (methoxy)(methyl)-phenylaminocarbonyl. Additional examples include imidazolyl, methylpyrazolyl, methylimidazolyl, methyloxadiazolyl, dimethylaminomethyl, N-acetyl-N-ethylamino, ethoxycarbonylamino, allyloxycarbonylamino, morpholinyl, dioxothiomorpholinyl, diethylaminocarbonyl, morpholinylcarbonyl and pyrrolidinylcarbonylmethyl.

Selected examples of specific substituents on the cyclic moiety of which M is the residue include one, two or three substituents independently selected from fluoro, methyl, ethyl, propyl, isopropyl, benzyl, imidazolyl, pyridinyl, methylpyrazolyl, methylimidazolyl, methyloxadiazolyl, methoxy, methoxymethyl, methylsulfonyl, oxo, acetyl, ethoxy-carbonyl, dimethylamino, dimethylaminomethyl, morpholinyl, dioxothiomorpholinyl, N-acetyl-N-ethylamino, ethoxycarbonylamino, allyloxycarbonylamino, aminocarbonyl, dimethylaminocarbonyl, diethylaminocarbonyl, (methoxy)(methyl)phenylaminocarbonyl, morpholinylcarbonyl and pyrrolidinylcarbonylmethyl.

Suitable examples of specific substituents on the cyclic moiety of which M is the residue include fluoro, methyl, ethyl, propyl, isopropyl, benzyl, pyridinyl, oxo, acetyl, ethoxycarbonyl and (methoxy)(methyl)phenylaminocarbonyl.

Typical values of the cyclic moiety of which M is the residue include 3,3-difluoro-azetidin-1-yl, pyrrolidin-1-yl, 3-hydroxypyrrolidin-1-yl, 3-(acetylamino)pyrrolidin-1-yl, 3-(hydroxyacetylamino)pyrrolidin-1-yl, imidazolidin-1-yl, 4-hydroxypiperidin-1-yl, 4-carboxypiperidin-1-yl, 4-(acetylamino)piperidin-1-yl, 4-(methylsulfonylamino)piperidin-1-yl, 4-(aminocarbonyl)piperidin-1-yl, 4-(methylaminocarbonyl)piperidin-1-yl, morpholin-4-yl, 3-methylmorpholin-4-yl, thiomorpholin-4-yl, 1,1-dioxothiomorpholin-4-yl, piperazin-1-yl, 4-methylpiperazin-1-yl, 4-ethylpiperazin-1-yl, 4-propylpiperazin-1-yl, 4-isopropylpiperazin-1-yl, 4-benzylpiperazin-1-yl, 4-(pyridin-2-yl)piperazin-1-yl, 4-(pyrazin-2-yl)piperazin-1-yl, 4-(methylsulfonyl)piperazin-1-yl, 4-(2-hydroxyethyl)-piperazin-1-yl, 3-oxopiperazin-1-yl, 4-methyl-3-oxopiperazin-1-yl, 4-acetylpiperazin-1-yl, 4-(ethylcarbonyl)piperazin-1-yl, 4-(tert-butylcarbonyl)piperazin-1-yl, 4-(hydroxyacetyl)piperazin-1-yl, 4-(dimethylaminoacetyl)piperazin-1-yl, 4-(carboxy-methyl)piperazin-1-yl, 4-(methoxycarbonyl)piperazin-1-yl, 4-(ethoxycarbonyl)piperazin-1-yl, 4-(ethoxycarbonylmethyl)piperazin-1-yl, 4-(aminocarbonyl)piperazin-1-yl, 4-(aminocarbonylmethyl)piperazin-1-yl, 4-(methylaminocarbonylmethyl)piperazin-1-yl, 4-(dimethylaminocarbonylmethyl)piperazin-1-yl, 4-[(4-methoxy-3-methylphenyl)amino-carbonyl]piperazin-1-yl, azepan-1-yl, 5-oxo-[1,4]diazepan-1-yl, 6-oxo-1,3,4,7,8,8a-hexahydropyrrolo[1,2-a]pyrazin-2-yl, 4,5,6,7-tetrahydropyrazolo[1,5-a]pyrazin-5-yl and 2-oxa-6-azaspiro[3.3]heptan-6-yl. Additional values include 3-methoxyazetidin-1-yl, 3-(methoxymethyl)azetidin-1-yl, 3-(dimethylaminomethyl)azetidin-1-yl, 3-(morpholin-4-yl)azetidin-1-yl, 3-(1,1-dioxothiomorpholin-4-yl)azetidin-1-yl, 3-(aminocarbonyl)-azetidin-1-yl, 3-(dimethylaminocarbonyl)azetidin-1-yl, 3-(imidazol-1-yl)pyrrolidin-1-yl, 3-(1-methylimidazol-2-yl)pyrrolidin-1-yl, 3-(methoxymethyl)pyrrolidin-1-yl, 3-(N-acetyl-N-ethylamino)pyrrolidin-1-yl, 3-(diethylaminocarbonyl)pyrrolidin-1-yl, 4-(2-methyl-pyrazol-3-yl)piperidin-1-yl, 4-methoxypiperidin-1-yl, 4-(dimethylamino)piperidin-1-yl, 4-(ethoxycarbonylamino)piperidin-1-yl, 4-(allyloxycarbonylamino)piperidin-1-yl, 3-(aminocarbonyl)piperidin-1-yl, 2-(1-methylpyrazol-4-yl)morpholin-4-yl, 2-(5-methyl-1,2,4-oxadiazol-3-yl)morpholin-4-yl, 4-(morpholin-4-ylcarbonyl)piperazin-1-yl, 4-(pyrrolidin-1-ylcarbonylmethyl)piperazin-1-yl, 4-methyl-[1,4]diazepan-1-yl, 4-acetyl-[1,4]diazepan-1-yl, 4-oxo-1,3,3a,5,6,6a-hexahydrocyclopenta[c]pyrrol-2-yl, 4-methyl-2,3,4a,5,7,7a-hexahydropyrrolo[3,4-b][1,4]oxazin-6-yl, 6,6-dimethyl-3-azabicyclo[3.1.0]-hexan-3-yl, 2-oxa-5-azabicyclo[2.2.1]heptan-5-yl and 3-oxo-8-azabicyclo[3.2.1]octan-8-yl.

Selected values of the cyclic moiety of which M is the residue include 3,3-difluoroazetidin-1-yl, 3-methoxyazetidin-1-yl, 3-(methoxymethyl)azetidin-1-yl, 3-(dimethylaminomethyl)azetidin-1-yl, 3-(morpholin-4-yl)azetidin-1-yl, 3-(1,1-dioxo-thiomorpholin-4-yl)azetidin-1-yl, 3-(aminocarbonyl)azetidin-1-yl, 3-(dimethylamino-carbonyl)azetidin-1-yl, 3-(imidazol-1-yl)pyrrolidin-1-yl, 3-(1-methylimidazol-2-yl)-pyrrolidin-1-yl, 3-(methoxymethyl)pyrrolidin-1-yl, 3-(N-acetyl-N-ethylamino)pyrrolidin-1-yl, 3-(diethylaminocarbonyl)pyrrolidin-1-yl, 4-(2-methylpyrazol-3-yl)piperidin-1-yl, 4-methoxypiperidin-1-yl, 4-(dimethylamino)piperidin-1-yl, 4-(ethoxycarbonylamino)-piperidin-1-yl, 4-(allyloxycarbonylamino)piperidin-1-yl, 3-(aminocarbonyl)piperidin-1-yl, 4-(aminocarbonyl)piperidin-1-yl, morpholin-4-yl, 2-(1-methylpyrazol-4-yl)morpholin-4-yl, 2-(5-methyl-1,2,4-oxadiazol-3-yl)morpholin-4-yl, 4-methylpiperazin-1-yl, 4-ethylpiperazin-1-yl, 4-propylpiperazin-1-yl, 4-isopropylpiperazin-1-yl, 4-benzylpiperazin-1-yl, 4-(pyridin-2-yl)piperazin-1-yl, 4-(methylsulfonyl)piperazin-1-yl, 3-oxopiperazin-1-yl, 4-acetylpiperazin-1-yl, 4-(ethoxycarbonyl)piperazin-1-yl, 4-[(4-methoxy-3-methyl-phenyl)aminocarbonyl]piperazin-1-yl, 4-(morpholin-4-ylcarbonyl)piperazin-1-yl, 4-(pyrrolidin-1-ylcarbonylmethyl)piperazin-1-yl, azepan-1-yl, 4-methyl-[1,4]diazepan-1-yl, 4-acetyl-[1,4]diazepan-1-yl, 4-oxo-1,3,3a,5,6,6a-hexahydrocyclopenta[c]pyrrol-2-yl, 4-methyl-2,3,4a,5,7,7a-hexahydropyrrolo[3,4-b][1,4]oxazin-6-yl, 6-oxo-1,3,4,7,8,8a-hexahydropyrrolo[1,2-a]pyrazin-2-yl, 6,6-dimethyl-3-azabicyclo[3.1.0]hexan-3-yl, 2-oxa-5-azabicyclo[2.2.1]heptan-5-yl, 3-oxo-8-azabicyclo[3.2.1]octan-8-yl and 2-oxa-6-aza-spiro[3.3]heptan-6-yl.

Suitable values of the cyclic moiety of which M is the residue include 3,3-difluoroazetidin-1-yl, morpholin-4-yl, 4-methylpiperazin-1-yl, 4-ethylpiperazin-1-yl, 4-propylpiperazin-1-yl, 4-isopropylpiperazin-1-yl, 4-benzylpiperazin-1-yl, 4-(pyridin-2-yl)-piperazin-1-yl, 4-acetylpiperazin-1-yl, 4-(ethoxycarbonyl)piperazin-1-yl, 4-[(4-methoxy-3-methylphenyl)aminocarbonyl]piperazin-1-yl, azepan-1-yl, 6-oxo-1,3,4,7,8,8a-hexahydropyrrolo[1,2-a]pyrazin-2-yl and 2-oxa-6-azaspiro[3.3]heptan-6-yl.

Suitably, R¹ represents hydrogen, halogen, cyano, nitro, hydroxy, trifluoromethyl, trifluoromethoxy, —OR^{a}, —SR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —NHSO₂NR^{b}R^{c}, —COR^{d}, —CO₂R^{d}, —CONR^{b}R^{c}, —CON(OR^{a})R^{b} or —SO₂NR^{b}R^{c}; or R¹ represents C₁₋₆ alkyl, aryl or heteroaryl, any of which groups may be optionally substituted by one or more substituents.

Typically, R¹ represents hydrogen, —OR^{a}, —SR^{a}, —SO₂R^{a}, —NR^{b}R^{c} or —NR^{c}COR^{d}; or R¹ represents C₁₋₆ alkyl, which group may be optionally substituted by one or more substituents.

Typical values of R¹ include hydrogen, —OR^{a}, —SR^{a}, —SO₂R^{a} and —NR^{b}R^{c}.

Suitable values of R¹ include hydrogen and —NR^{b}R^{c}.

In a first embodiment, R¹ represents hydrogen. In a second embodiment, R¹ represents cyano. In a third embodiment, R¹ represents —OR^{a}. In a fourth embodiment, R¹ represents —SR^{a}. In a fifth embodiment, R¹ represents —SO₂R^{a}. In a sixth embodiment, R¹ represents —NR^{b}R^{c}. In a seventh embodiment, R¹ represents —NR^{c}COR^{d}. In an eighth embodiment, R¹ represents optionally substituted C₁₋₆ alkyl. In one aspect of that embodiment, R¹ represents optionally substituted methyl.

Examples of typical substituents on R¹ include one or more substituents independently selected from halogen, cyano, nitro, C₁₋₆ alkyl, trifluoromethyl, aryl(C₁₋₆)alkyl, hydroxy, C₁₋₆ alkoxy, difluoromethoxy, trifluoromethoxy, aryloxy, C₁₋₄ alkylenedioxy, C₁₋₆ alkoxy(C₁₋₆)alkyl, C₁₋₆ alkylthio, C₁₋₆ alkylsulfonyl, oxo, amino, C₁₋₆ alkylamino, di(C₁₋₆)alkylamino, C₂₋₆ alkylcarbonylamino, C₂₋₆ alkoxycarbonylamino, aryl(C₁₋₆)alkoxycarbonylamino, C₁₋₆ alkylaminocarbonylamino, arylaminocarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Specific examples of typical substituents on R¹ include one or more substituents independently selected from fluoro, chloro, bromo, cyano, nitro, methyl, ethyl, tert-butyl, trifluoromethyl, benzyl, hydroxy, methoxy, difluoromethoxy, trifluoromethoxy, phenoxy, methylenedioxy, ethylenedioxy, methoxymethyl, methylthio, methylsulfonyl, oxo, amino, methylamino, dimethylamino, acetylamino, methoxycarbonylamino, ethoxycarbonyl-amino, benzyloxycarbonylamino, ethylaminocarbonylamino, butylaminocarbonylamino, phenylaminocarbonylamino, methylsulfonylamino, formyl, acetyl, carboxy, methoxycarbonyl, aminocarbonyl, methylaminocarbonyl, dimethylaminocarbonyl, aminosulfonyl, methylaminosulfonyl and dimethylaminosulfonyl.

Generally, R² represents hydrogen, cyano, hydroxy, trifluoromethyl, —NR^{c}CO₂R^{d}, —COR^{d}, —CO₂R^{d}, —CONR^{b}R^{c} or —CON(OR^{a})R^{b}; or R² represents C₁₋₆ alkyl, C₃₋₇ cycloalkyl, aryl, C₃₋₇ heterocycloalkyl, C₃₋₇ heterocycloalkenyl or heteroaryl, any of which groups may be optionally substituted by one or more substituents.

Typically, R² represents hydrogen; or R² represents aryl, C₃₋₇ heterocycloalkyl or heteroaryl, any of which groups may be optionally substituted by one or more substituents.

Appositely, R² is other than hydrogen.

Suitably, R² represents aryl or heteroaryl, either of which groups may be optionally substituted by one or more substituents.

In a first embodiment, R² represents hydrogen. In a second embodiment, R² represents cyano. In a third embodiment, R² represents hydroxy. In a fourth embodiment, R² represents trifluoromethyl. In a fifth embodiment, R² represents —NR^{c}CO₂R^{d}. In a sixth embodiment, R² represents —COR^{d}. In a seventh embodiment, R² represents —CO₂R^{d}. In an eighth embodiment, R² represents —CONR^{b}R^{c}. In a ninth embodiment, R² represents —CON(OR^{a})R^{b}. In a tenth embodiment, R² represents optionally substituted C₁₋₆ alkyl. In a first aspect of that embodiment, R² represents unsubstituted C₁₋₆ alkyl. In a second aspect of that embodiment, R² represents monosubstituted C₁₋₆ alkyl. In a third aspect of that embodiment, R² represents disubstituted C₁₋₆ alkyl. In an eleventh embodiment, R² represents optionally substituted C₃₋₇ cycloalkyl. In a first aspect of that embodiment, R² represents unsubstituted C₃₋₇ cycloalkyl. In a second aspect of that embodiment, R² represents monosubstituted C₃₋₇ cycloalkyl. In a third aspect of that embodiment, R² represents disubstituted C₃₋₇ cycloalkyl. In a twelfth embodiment, R² represents optionally substituted aryl. In a first aspect of that embodiment, R² represents unsubstituted aryl. In a second aspect of that embodiment, R² represents monosubstituted aryl. In a third aspect of that embodiment, R² represents disubstituted aryl. In a thirteenth embodiment, R² represents optionally substituted C₃₋₇ heterocycloalkyl. In a first aspect of that embodiment, R² represents unsubstituted C₃₋₇ heterocycloalkyl. In a second aspect of that embodiment, R² represents monosubstituted C₃₋₇ heterocycloalkyl. In a third aspect of that embodiment, R² represents disubstituted C₃₋₇ heterocycloalkyl. In a fourteenth embodiment, R² represents optionally substituted C₃₋₇ heterocycloalkenyl. In a first aspect of that embodiment, R² represents unsubstituted C₃₋₇ heterocycloalkenyl. In a second aspect of that embodiment, R² represents monosubstituted C₃₋₇ heterocycloalkenyl. In a third aspect of that embodiment, R² represents disubstituted C₃₋₇ heterocycloalkenyl. In a fifteenth embodiment, R² represents optionally substituted heteroaryl. In a first aspect of that embodiment, R² represents unsubstituted heteroaryl. In a second aspect of that embodiment, R² represents monosubstituted heteroaryl. In a third aspect of that embodiment, R² represents disubstituted heteroaryl.

Where R² represents optionally substituted C₁₋₆ alkyl, suitable values include methyl, ethyl, n-propyl, isopropyl, n-butyl, isobutyl and tert-butyl, any of which groups may be optionally substituted by one or more substituents. Selected values include methyl, hydroxymethyl, chloropropyl and isobutyl. Particular values include methyl and isobutyl, especially methyl.

Where R² represents optionally substituted C₃₋₇ cycloalkyl, a suitable value is cyclohexyl, optionally substituted by one or more substituents.

Where R² represents optionally substituted aryl, a suitable value is phenyl, optionally substituted by one or more substituents.

Where R² represents optionally substituted C₃₋₇ heterocycloalkyl, typical values include azetidinyl, dihydroisobenzofuranyl, pyrrolidinyl, indolinyl, piperidinyl, piperazinyl, morpholinyl and thiomorpholinyl, any of which groups may be optionally substituted by one or more substituents. Suitable values include dihydroisobenzofuranyl and indolinyl, either of which groups may be optionally substituted by one or more substituents.

Where R² represents optionally substituted C₃₋₇ heterocycloalkenyl, a typical value is oxazolinyl, optionally substituted by one or more substituents. Suitable values include oxazolinyl, methyloxazolinyl, isopropyloxazolinyl and dimethyloxazolinyl.

Where R² represents optionally substituted heteroaryl, typical values include furyl, thienyl, pyrrolyl, pyrazolyl, indazolyl, oxazolyl, isoxazolyl, thiazolyl, isothiazolyl, imidazolyl, imidazo[1,5-a]pyridinyl, oxadiazolyl, benzoxadiazolyl, thiadiazolyl, triazolyl, [1,2,4]triazolo[4,3-a]pyridinyl, tetrazolyl, pyridinyl, pyridazinyl, pyrimidinyl, pyrazinyl and triazinyl, any of which groups may be optionally substituted by one or more substituents. Suitable values include indazolyl, imidazo[1,5-a]pyridinyl, benzoxadiazolyl, [1,2,4]triazolo[4,3-a]pyridinyl and pyridinyl, any of which groups may be optionally substituted by one or more substituents.

In a typical embodiment, R² represents hydrogen; or R² represents phenyl, dihydroisobenzofuranyl, indolinyl, indazolyl, imidazo[1,5-a]pyridinyl, benzoxadiazolyl, [1,2,4]triazolo[4,3-a]pyridinyl or pyridinyl, any of which groups may be optionally substituted by one or more substituents.

Typical examples of optional substituents on R² include one or more substituents independently selected from halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, oxo, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylamino-carbonyl, aminosulfonyl, C₁₋₆ alkylaminosulfonyl and di(C₁₋₆)alkylaminosulfonyl.

Suitable examples of optional substituents on R² include one or more substituents independently selected from halogen, C₁₋₆ alkyl, C₁₋₆ alkoxy, difluoromethoxy, C₁₋₆ alkylsulfonyl, oxo and C₁₋₆ alkylaminocarbonyl.

Typical examples of specific substituents on R² include one or more substituents independently selected from fluoro, chloro, bromo, cyano, nitro, methyl, ethyl, isopropyl, tert-butyl, trifluoromethyl, hydroxy, methoxy, isopropoxy, difluoromethoxy, trifluoro-methoxy, methylthio, methylsulfinyl, methylsulfonyl, oxo, amino, methylamino, dimethylamino, acetylamino, methoxycarbonylamino, methylsulfonylamino, formyl, acetyl, carboxy, methoxycarbonyl, aminocarbonyl, methylaminocarbonyl, dimethyl-aminocarbonyl, aminosulfonyl, methylaminosulfonyl and dimethylaminosulfonyl.

Suitable examples of specific substituents on R² include one or more substituents independently selected from fluoro, chloro, methyl, methoxy, difluoromethoxy, methyl-sulfonyl, oxo and methylaminocarbonyl.

Typical values of R² include hydrogen, cyano, hydroxy, trifluoromethyl, —NR^{c}CO₂R^{d}, —COR^{d}, —CO₂R^{d}, —CONR^{b}R^{c}, —CON(OR^{a})R^{b}, methyl, hydroxymethyl, chloro-propyl, isobutyl, cyclohexyl, phenyl, fluorophenyl, chlorophenyl, methoxyphenyl, (fluoro)(methoxy)phenyl, dimethoxyphenyl, (difluoromethoxy)(methoxy)phenyl, (methoxy)(methylsulfonyl)phenyl, (chloro)(methylaminocarbonyl)phenyl, oxo-3H-isobenzofuranyl, (methyl)(oxo)indolinyl, oxazolinyl, methyloxazolinyl, isopropyl-oxazolinyl, dimethyloxazolinyl, methylindazolyl, dimethylindazolyl, dimethylimidazo-[1,5-a]pyridinyl, methyloxadiazolyl, isopropyloxadiazolyl, tert-butyloxadiazolyl, benzoxadiazolyl, methyl[1,2,4]triazolo[4,3-a]pyridinyl, pyridinyl and dimethoxy-pyridinyl.

Suitable values of R² include hydrogen, (fluoro)(methoxy)phenyl, dimethoxy-phenyl, (difluoromethoxy)(methoxy)phenyl, (methoxy)(methylsulfonyl)phenyl, (chloro)-(methylaminocarbonyl)phenyl, oxo-3H-isobenzofuranyl, (methyl)(oxo)indolinyl, methyl-indazolyl, dimethylindazolyl, dimethylimidazo[1,5-a]pyridinyl, benzoxadiazolyl, methyl-[1,2,4]triazolo[4,3-a]pyridinyl and dimethoxypyridinyl.

Typically, R³ represents hydrogen or C₁₋₆ alkyl.

In a first embodiment, R³ represents hydrogen. In a second embodiment, R³ represents halogen, especially fluoro or chloro. In a first aspect of that embodiment, R³ represents fluoro. In a second aspect of that embodiment, R³ represents chloro. In a third embodiment, R³ represents cyano. In a fourth embodiment, R³ represents trifluoromethyl. In a fifth embodiment, R³ represents C₁₋₆ alkyl, especially methyl.

Typical values of R³ include hydrogen and methyl.

Typical examples of suitable substituents on R^{a}, R^{b}, R^{c}, R^{d} or R^{e}, or on the heterocyclic moiety —NR^{b}R^{c}, include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulfinyl, C₁₋₆ alkylsulfonyl, C₁₋₆ alkylsulfonimidoyl, N,S-di(C₁₋₆)alkylsulfonimidoyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, C₂₋₆ alkylcarbonyloxy, amino, C₁₋₆ alkylamino, di-(C₁₋₆)alkylamino, phenylamino, pyridinylamino, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, aminocarbonyl, C₁₋₆ alkylaminocarbonyl and di(C₁₋₆)alkylaminocarbonyl.

Typical examples of specific substituents on R^{a}, R^{b}, R^{c}, R^{d} or R^{e}, or on the heterocyclic moiety —NR^{b}R^{c}, include fluoro, chloro, bromo, methyl, ethyl, isopropyl, methoxy, isopropoxy, difluoromethoxy, trifluoromethoxy, methoxymethyl, methylthio, ethylthio, methylsulfinyl, methylsulfonyl, methylsulfonimidoyl, N,S-dimethyl-sulfonimidoyl, hydroxy, hydroxymethyl, hydroxyethyl, aminomethyl, cyano, trifluoro-methyl, oxo, acetyl, carboxy, methoxycarbonyl, ethoxycarbonyl, tert-butoxycarbonyl, acetoxy, amino, methylamino, ethylamino, dimethylamino, phenylamino, pyridinylamino, acetylamino, acetylaminomethyl, tert-butoxycarbonylamino, methylsulfonylamino, aminocarbonyl, methylaminocarbonyl and dimethylaminocarbonyl.

Typically, R^{a} represents hydrogen; or R^{a} represents C₁₋₆ alkyl, aryl(C₁₋₆)alkyl or heteroaryl(C₁₋₆)alkyl, any of which groups may be optionally substituted by one or more substituents.

Suitably, R^{a} represents C₁₋₆ alkyl, aryl(C₁₋₆)alkyl or heteroaryl(C₁₋₆)alkyl, any of which groups may be optionally substituted by one or more substituents.

Apposite values of R^{a} include hydrogen; and methyl, ethyl, benzyl or isoindolyl-propyl, any of which groups may be optionally substituted by one or more substituents.

Selected values of R^{a} include methyl, ethyl, benzyl and isoindolylpropyl, any of which groups may be optionally substituted by one or more substituents.

Selected examples of suitable substituents on R^{a} include C₁₋₆ alkoxy and oxo.

Selected examples of specific substituents on R^{a} include methoxy and oxo.

In one embodiment, R^{a} represents hydrogen. In another embodiment, R^{a} represents optionally substituted C₁₋₆ alkyl. In one aspect of that embodiment, R^{a} ideally represents unsubstituted C₁₋₆ alkyl, especially methyl. In another aspect of that embodiment, R^{a} ideally represents substituted C₁₋₆ alkyl, e.g. methoxyethyl. In another embodiment, R^{a} represents optionally substituted aryl. In one aspect of that embodiment, R^{a} represents unsubstituted aryl, especially phenyl. In another aspect of that embodiment, R^{a} represents monosubstituted aryl, especially methylphenyl. In another embodiment, R^{a} represents optionally substituted aryl(C₁₋₆)alkyl, ideally unsubstituted aryl(C₁₋₆)alkyl, especially benzyl. In a further embodiment, R^{a} represents optionally substituted heteroaryl. In a further embodiment, R^{a} represents optionally substituted heteroaryl(C₁₋₆)alkyl, e.g. dioxoisoindolylpropyl.

Specific values of R^{a} include methyl, methoxyethyl, benzyl and dioxoisoindolyl-propyl.

Appositely, R^{a} represents hydrogen or C₁₋₆ alkyl.

Individual values of R^{a} include hydrogen and methyl.

In a typical aspect, R^{b} represents hydrogen or trifluoromethyl; or R^{b} represents C₁₋₆ alkyl, C₃₋₇ cycloalkyl, C₃₋₇ cycloalkyl(C₁₋₆)alkyl, aryl, aryl(C₁₋₆)alkyl, C₃₋₇ hetero-cycloalkyl, C₃₋₇ heterocycloalkyl(C₁₋₆)alkyl, heteroaryl or heteroaryl(C₁₋₆)alkyl, any of which groups may be optionally substituted by one or more substituents.

In a suitable aspect, R^{b} represents hydrogen; or R^{b} represents aryl(C₁₋₆)alkyl or heteroaryl(C₁₋₆)alkyl, either of which groups may be optionally substituted by one or more substituents.

Illustratively, R^{b} represents hydrogen or trifluoromethyl; or R^{b} represents methyl, ethyl, n-propyl, isopropyl, n-butyl, 2-methylpropyl, tert-butyl, pentyl, hexyl, cyclopropyl, cyclobutyl, cyclopentyl, cyclohexyl, cyclopropylmethyl, cyclobutylmethyl, cyclopentyl-methyl, cyclohexylmethyl, phenyl, benzyl, phenylethyl, azetidinyl, tetrahydrofuryl, tetrahydrothienyl, pyrrolidinyl, piperidinyl, homopiperidinyl, morpholinyl, azetidinylmethyl, tetrahydrofurylmethyl, pyrrolidinylmethyl, pyrrolidinylethyl, pyrrolidinylpropyl, thiazolidinylmethyl, imidazolidinylethyl, piperidinylmethyl, piperidinylethyl, tetrahydroquinolinylmethyl, piperazinylpropyl, morpholinylmethyl, morpholinylethyl, morpholinylpropyl, pyridinyl, indolylmethyl, isoxazolylmethyl, thiazolylmethyl, pyrazolylmethyl, pyrazolylethyl, imidazolylmethyl, imidazolylethyl, benzimidazolylmethyl, oxadiazolylmethyl, triazolylmethyl, pyridinylmethyl or pyridinylethyl, any of which groups may be optionally substituted by one or more substituents.

Suitably, R^{b} represents hydrogen; or R^{b} represents benzyl, isoxazolylmethyl, thiazolylmethyl, pyrazolylmethyl, oxadiazolylmethyl or pyridinylmethyl, any of which groups may be optionally substituted by one or more substituents.

Typical examples of optional substituents on R^{b} include C₁₋₆ alkyl, C₁₋₆ alkoxy, C₁₋₆ alkylthio, C₁₋₆ alkylsulfinyl, C₁₋₆ alkylsulfonyl, C₁₋₆ alkylsulfonimidoyl, N,S-di-(C₁₋₆)alkylsulfonimidoyl, hydroxy, cyano, C₂₋₆ alkoxycarbonyl, di(C₁₋₆)alkylamino and C₂₋₆ alkoxycarbonylamino.

Suitable examples of optional substituents on R^{b} include C₁₋₆ alkyl, C₁₋₆ alkyl-sulfonyl, C₁₋₆ alkylsulfonimidoyl and N,S-di(C₁₋₆)alkylsulfonimidoyl.

Typical examples of specific substituents on R^{b} include methyl, methoxy, methylthio, methylsulfinyl, methylsulfonyl, methylsulfonimidoyl, N,S-dimethyl-sulfonimidoyl, hydroxy, cyano, tert-butoxycarbonyl, dimethylamino and tert-butoxycarbonylamino.

Suitable examples of specific substituents on R^{b} include methyl, methylsulfonyl, methylsulfonimidoyl and N,S-dimethylsulfonimidoyl.

Typical values of R^{b} include hydrogen, methyl, methoxyethyl, methylthioethyl, methylsulfinylethyl, methylsulfonylethyl, hydroxyethyl, cyanoethyl, dimethylaminoethyl, tert-butoxycarbonylaminoethyl, dihydroxypropyl, benzyl, methylsulfonylbenzyl, methyl-sulfonimidoylbenzyl, N,S-dimethylsulfonimidoylbenzyl, pyrrolidinyl, tert-butoxycarbonyl-pyrrolidinyl, morpholinylpropyl, methylisoxazolylmethyl, dimethylthiazolylmethyl, dimethylpyrazolylmethyl, methyloxadiazolylmethyl and methylpyridinylmethyl.

Suitable values of R^{b} include hydrogen, methylsulfonylbenzyl, methyl-sulfonimidoylbenzyl, N,S-dimethylsulfonimidoylbenzyl, methylisoxazolylmethyl, dimethylthiazolylmethyl, dimethylpyrazolylmethyl, methyloxadiazolylmethyl and methylpyridinylmethyl.

In one embodiment, R^{b} represents hydrogen. In another embodiment, R^{b} is other than hydrogen.

Selected values of R^{c} include hydrogen; or C₁₋₆ alkyl, C₃₋₇ cycloalkyl or C₃₋₇ heterocycloalkyl, any of which groups may be optionally substituted by one or more substituents.

In a particular aspect, R^{c} represents hydrogen, C₁₋₆ alkyl or C₃₋₇ cycloalkyl.

Representative values of R^{c} include hydrogen; or methyl, cyclobutyl, cyclopentyl, cyclohexyl, tetrahydropyranyl and piperidinyl, any of which groups may be optionally substituted by one or more substituents.

Selected examples of suitable substituents on R^{c} include C₂₋₆ alkylcarbonyl and C₂₋₆ alkoxycarbonyl.

Selected examples of specific substituents on R^{c} include acetyl and tert-butoxycarbonyl.

Specific values of R^{c} include hydrogen, methyl, cyclobutyl, cyclopentyl, cyclohexyl, tetrahydropyranyl, acetylpiperidinyl and tert-butoxycarbonylpiperidinyl.

Suitably, R^{c} represents hydrogen or C₁₋₆ alkyl. In one embodiment, R^{c} is hydrogen. In another embodiment, R^{c} represents C₁₋₆ alkyl, especially methyl or ethyl, particularly methyl. In a further embodiment, R^{c} represents C₃₋₇ cycloalkyl, e.g. cyclopropyl, cyclobutyl, cyclopentyl or cyclohexyl.

Alternatively, the moiety —NR^{b}R^{c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{b}R^{c} include C₁₋₆ alkyl, C₁₋₆ alkylsulfonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonyl-amino, C₂₋₆ alkylcarbonylamino(C₁₋₆)alkyl, C₂₋₆ alkoxycarbonylamino, C₁₋₆ alkyl-sulfonylamino and aminocarbonyl.

Selected examples of specific substituents on the heterocyclic moiety —NR^{b}R^{c} include methyl, methylsulfonyl, hydroxy, hydroxymethyl, aminomethyl, cyano, oxo, acetyl, carboxy, ethoxycarbonyl, amino, acetylamino, acetylaminomethyl, tert-butoxy-carbonylamino, methylsulfonylamino and aminocarbonyl.

Specific values of the moiety —NR^{b}R^{c} include azetidin-1-yl, hydroxyazetidin-1-yl, hydroxymethylazetidin-1-yl, (hydroxy)(hydroxymethyl)azetidin-1-yl, aminomethyl-azetidin-1-yl, cyanoazetidin-1-yl, carboxyazetidin-1-yl, aminoazetidin-1-yl, aminocarbonylazetidin-1-yl, pyrrolidin-1-yl, aminomethylpyrrolidin-1-yl, oxopyrrolidin-1-yl, acetylaminomethylpyrrolidin-1-yl, tert-butoxycarbonylaminopyrrolidin-1-yl, oxo-oxazolidin-3-yl, hydroxyisoxazolidin-2-yl, thiazolidin-3-yl, oxothiazolidin-3-yl, dioxo-isothiazolidin-2-yl, piperidin-1-yl, hydroxypiperidin-1-yl, hydroxymethylpiperidin-1-yl, aminopiperidin-1-yl, acetylaminopiperidin-1-yl, tert-butoxycarbonylaminopiperidin-1-yl, methylsulfonylaminopiperidin-1-yl, morpholin-4-yl, piperazin-1-yl, methylpiperazin-1-yl, methylsulfonylpiperazin-1-yl, oxopiperazin-1-yl, acetylpiperazin-1-yl, ethoxycarbonyl-piperazin-1-yl and oxohomopiperazin-1-yl.

Suitably, R^{d} represents hydrogen; or C₁₋₆ alkyl, aryl or heteroaryl, any of which groups may be optionally substituted by one or more substituents.

Selected examples of suitable values for R^{d} include hydrogen, methyl, ethyl, isopropyl, 2-methylpropyl, tert-butyl, cyclopropyl, cyclobutyl, phenyl, thiazolidinyl, thienyl, imidazolyl and thiazolyl, any of which groups may be optionally substituted by one or more substituents.

Selected examples of suitable substituents on R^{d} include halogen, C₁₋₆ alkyl, C₁₋₆ alkoxy, oxo, C₂₋₆ alkylcarbonyloxy and di(C₁₋₆)alkylamino.

Selected examples of particular substituents on R^{d} include fluoro, methyl, methoxy, oxo, acetoxy and dimethylamino.

In one embodiment, R^{d} represents hydrogen. In another embodiment, R^{d} represents optionally substituted C₁₋₆ alkyl. In one aspect of that embodiment, R^{d} ideally represents unsubstituted C₁₋₆ alkyl, e.g. methyl, ethyl, isopropyl, 2-methylpropyl or tert-butyl, especially methyl or ethyl, particularly methyl. In another aspect of that embodiment, R^{d} ideally represents substituted C₁₋₆ alkyl, e.g. substituted methyl or substituted ethyl, including acetoxymethyl, dimethylaminomethyl and trifluoroethyl. In another embodiment, R^{d} represents optionally substituted aryl. In one aspect of that embodiment, R^{d} represents unsubstituted aryl, especially phenyl. In another aspect of that embodiment, R^{d} represents monosubstituted aryl, especially methylphenyl. In a further aspect of that embodiment, R^{d} represents disubstituted aryl, e.g. dimethoxyphenyl. In a further embodiment, R^{d} represents optionally substituted heteroaryl, e.g. thienyl, chlorothienyl, methylthienyl, methylimidazolyl or thiazolyl. In another embodiment, R^{d} represents optionally substituted C₃₋₇ cycloalkyl, e.g. cyclopropyl or cyclobutyl. In a further embodiment, R^{d} represents optionally substituted C₃₋₇ heterocycloalkyl, e.g. thiazolidinyl or oxothiazolidinyl.

Selected examples of specific values for R^{d} include hydrogen, methyl, ethyl, acetoxymethyl, dimethylaminomethyl, ethyl, trifluoroethyl, isopropyl, 2-methylpropyl, tert-butyl, cyclopropyl, cyclobutyl, phenyl, dimethoxyphenyl, thiazolidinyl, oxothiazolidinyl, thienyl, chlorothienyl, methylthienyl, methylimidazolyl and thiazolyl.

Appositely, R^{d} represents hydrogen or C₁₋₆ alkyl.

Individual values of R^{d} include hydrogen, methyl and ethyl.

A particular value of R^{d} is ethyl.

Suitably, R^{e} represents C₁₋₆ alkyl or aryl, either of which groups may be optionally substituted by one or more substituents.

Selected examples of suitable substituents on R^{e} include C₁₋₆ alkyl, especially methyl.

In one embodiment, R^{e} represents optionally substituted C₁₋₆ alkyl, ideally unsubstituted C₁₋₆ alkyl, e.g. methyl or propyl, especially methyl. In another embodiment, R^{e} represents optionally substituted aryl. In one aspect of that embodiment, R^{e} represents unsubstituted aryl, especially phenyl. In another aspect of that embodiment, R^{e} represents monosubstituted aryl, especially methylphenyl. In a further embodiment, R^{e} represents optionally substituted heteroaryl.

Selected values of R^{e} include methyl, propyl and methylphenyl.

One sub-class of compounds according to the invention is represented by the compounds of formula (IIA), and pharmaceutically acceptable salts and solvates thereof:

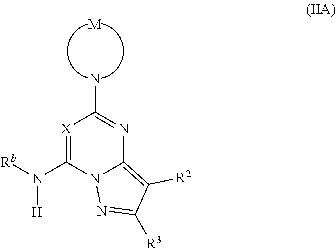

wherein

X, M, R², R³ and R^{b} are as defined above.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments. These include inflammatory, autoimmune and oncological disorders; viral diseases and malaria; and organ and cell transplant rejection.

Inflammatory and autoimmune disorders include systemic autoimmune disorders, autoimmune endocrine disorders and organ-specific autoimmune disorders. Systemic autoimmune disorders include systemic lupus erythematosus (SLE), psoriasis, vasculitis, polymyositis, scleroderma, multiple sclerosis, ankylosing spondylitis, rheumatoid arthritis and Sjogren's syndrome. Autoimmune endocrine disorders include thyroiditis. Organ-specific autoimmune disorders include Addison's disease, haemolytic or pernicious anaemia, glomerulonephritis (including Goodpasture's syndrome), Graves' disease, idiopathic thrombocytopenic purpura, insulin-dependent diabetes mellitus, juvenile diabetes, uveitis, inflammatory bowel disease (including Crohn's disease and ulcerative colitis), pemphigus, atopic dermatitis, autoimmune hepatitis, primary biliary cirrhosis, autoimmune pneumonitis, autoimmune carditis, myasthenia gravis and spontaneous infertility.

Oncological disorders, which may be acute or chronic, include proliferative disorders, especially cancer, in animals, including mammals, especially humans. Particular categories of cancer include haematological malignancy (including leukaemia and lymphoma) and non-haematological malignancy (including solid tumour cancer, sarcoma, meningioma, glioblastoma multiforme, neuroblastoma, melanoma, gastric carcinoma and renal cell carcinoma). Chronic leukaemia may be myeloid or lymphoid. Varieties of leukaemia include lymphoblastic T cell leukaemia, chronic myelogenous leukaemia (CML), chronic lymphocytic/lymphoid leukaemia (CLL), hairy-cell leukaemia, acute lymphoblastic leukaemia (ALL), acute myelogenous leukaemia (AML), myelodysplastic syndrome, chronic neutrophilic leukaemia, acute lymphoblastic T cell leukaemia, plasmacytoma, immunoblastic large cell leukaemia, mantle cell leukaemia, multiple myeloma, acute megakaryoblastic leukaemia, acute megakaryocytic leukaemia, promyelocytic leukaemia and erythroleukaemia. Varieties of lymphoma include malignant lymphoma, Hodgkin's lymphoma, non-Hodgkin's lymphoma, lymphoblastic T cell lymphoma, Burkitt's lymphoma, follicular lymphoma, MALT1 lymphoma and marginal zone lymphoma. Varieties of non-haematological malignancy include cancer of the prostate, lung, breast, rectum, colon, lymph node, bladder, kidney, pancreas, liver, ovary, uterus, cervix, brain, skin, bone, stomach and muscle.

Viral diseases include infections caused by various families of virus, including the Retroviridae, Flaviviridae, Picornaviridae. Various genera within the Retroviridae family include Alpharetrovirus, Betaretrovirus, Gammaretrovirus, Deltaretrovirus, Epsilonretrovirus, Lentivirus and Spumavirus. Members of the Lentivirus genus include human immunodeficiency virus 1 (HIV-1) and human immunodeficiency virus 2 (HIV-2). Various genera within the Flaviviridae family include Flavivirus, Pestivirus, Hepacivirus and Hepatitis G Virus. Members of the Flavivirus genus include Dengue fever virus, yellow fever virus, West Nile encephalitis virus and Japanese encephalitis virus. Members of the Pestivirus genus include bovine viral diarrhoea virus (BVDV), classical swine fever virus and border disease virus 2 (BDV-2). Members of the Hepacivirus genus include hepatitis C virus (HCV). Members of the Hepatitis G Virus genus include hepatitis G virus. Various genera within the Picornaviridae family include Aphthovirus, Avihepatovirus, Cardiovirus, Enterovirus, Erbovirus, Hepatovirus, Kobuvirus, Parechovirus, Sapelovirus, Senecavirus, Teschovirus and Tremovirus. Members of the Enterovirus genus include poliovirus, coxsackie A virus, coxsackie B virus and rhinovirus.

Organ transplant rejection includes the rejection of transplanted or grafted organs or cells (both allografts and xenografts), including graft-versus-host reaction disease. The term “organ” as used herein means all organs or parts of organs in mammals, particularly humans, including kidney, lung, bone marrow, hair, cornea, eye (vitreous), heart, heart valve, liver, pancreas, blood vessel, skin, muscle, bone, intestine and stomach. The term “rejection” as used herein means all reactions of the recipient body or the transplanted organ which ultimately lead to cell or tissue death in the transplanted organ, or adversely affect the functional ability and viability of the transplanted organ or the recipient. In particular, this means acute and chronic rejection reactions.

Cell transplant rejection includes the rejection of cell transplants and xeno-transplantation. The major hurdle for xenotransplantation is that even before the T lymphocytes (responsible for the rejection of allografts) are activated, the innate immune system (especially T-independent B lymphocytes and macrophages) is activated. This provokes two types of severe and early acute rejection, referred to as hyperacute rejection and vascular rejection respectively. Conventional immunosuppressant drugs, including cyclosporine A, are ineffective in xenotransplantation. The compounds in accordance with the present invention are not liable to this drawback. The ability of the compounds of this invention to suppress T-independent xeno-antibody production as well as macrophage activation may be demonstrated by their ability to prevent xenograft rejection in athymic, T-deficient mice receiving xenogenic hamster-heart grafts.

The present invention also provides a pharmaceutical composition which comprises a compound in accordance with the invention as described above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

The compounds of formula (I) above may be prepared by a process which comprises reacting a compound of formula (III) with a compound of formula (IV):

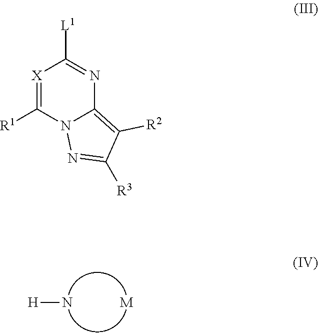

wherein X, M, R¹, R² and R³ are as defined above, and L¹ represents a suitable leaving group.

The leaving group L¹ is typically a halogen atom, e.g. chloro. Alternatively, the leaving group L¹ may be C₁₋₆ alkylsulfanyl, e.g. methylsulfanyl, or C₁₋₆ alkylsulfonyl, e.g. methylsulfonyl.

The reaction is conveniently effected at an elevated temperature in a suitable solvent, e.g. an organic nitrile such as acetonitrile, a lower alkanol such as ethanol, isopropanol or n-butanol, an ethereal solvent such as tetrahydrofuran or 1,4-dioxane, or an organic amide such as N,N-dimethylacetamide. The reaction may be performed in the presence of a suitable base, e.g. an organic base such as N,N-diisopropylethylamine.

The intermediates of formula (III) where X represents N, R¹ represents —NH₂ and L¹ represents methylsulfanyl may be prepared by reacting dimethyl N-cyanodithioimino-carbonate with a compound of formula (V):

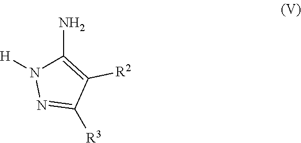

wherein R² and R³ are as defined above.

The reaction is conveniently effected at an elevated temperature in a suitable solvent, e.g. a lower alkanol such as ethanol, typically in the presence of an organic base such as piperidine.

The compounds of formula (I) above, wherein R² represents optionally substituted aryl or optionally substituted heteroaryl, may be prepared by a process which comprises reacting a compound of formula R^{2a}—B¹ with a compound of formula (VI):

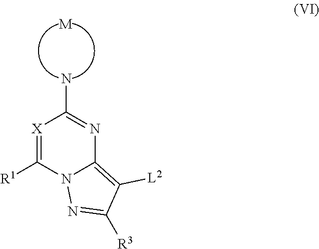

wherein X, M, R¹ and R³ are as defined above, R^{2a} represents optionally substituted aryl or optionally substituted heteroaryl, L² represents a suitable leaving group, and B¹ represents a boronic acid moiety —B(OH)₂ or a cyclic ester thereof formed with an organic diol, e.g. pinacol, 1,3-propanediol or neopentyl glycol; in the presence of a transition metal catalyst.

The leaving group L² is typically a halogen atom, e.g. bromo or iodo.

The transition metal catalyst of use in the reaction between the compound of formula R^{2a}—B¹ and compound (VI) is suitably a palladium-containing catalyst such as tetrakis(triphenylphosphine)palladium(0) or dichloro[1,1′-bis(diphenylphosphino)-ferrocene]palladium(II).

The reaction is conveniently carried out at an elevated temperature in a suitable solvent, e.g. an ethereal solvent such as 1,4-dioxane or 1,2-dimethoxyethane, typically in the presence of potassium phosphate, potassium carbonate or sodium carbonate.

The intermediates of formula (VI) may be prepared by reacting a compound of formula (IV) as defined above with a compound of formula (VII):

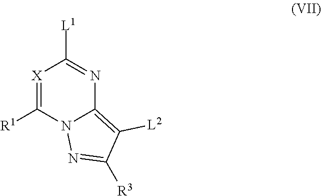

wherein X, R¹, R³, L¹ and L² are as defined above; under conditions analogous to those described above for the reaction between compounds (III) and (IV).

An intermediate of formula (III) or (VII) wherein L¹ represents C₁₋₆ alkylsulfanyl, e.g. methylsulfanyl, may be converted into the corresponding compound wherein L¹ represents C₁₋₆ alkylsulfonyl, e.g. methylsulfonyl, by treatment with a suitable oxidising agent, e.g. 3-chloroperoxybenzoic acid.

The intermediates of formula (VII) wherein R¹ represents —NR^{b}R^{c} may be prepared by reacting a compound of formula H—NR^{b}R^{c} with a compound of formula (VIII):

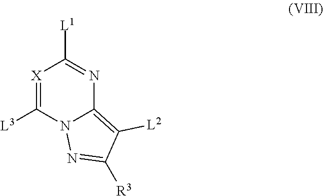

wherein X, R³, R^{b}, R^{c}, L¹ and L² are as defined above, and L³ represents a suitable leaving group.

The leaving group L³ is typically a halogen atom, e.g. chloro.

The reaction is conveniently effected at an elevated temperature in a suitable solvent, e.g. a lower alkanol such as isopropanol or n-butanol. The reaction may be performed in the presence of a suitable base, e.g. an organic base such as N,N-diisopropylethylamine. By analogy, where R^{b} and R^{c} are both H, the reaction may conveniently be performed by treating compound (VIII) with aqueous ammonia, or aqueous ammonium hydroxide solution, in a suitable solvent, e.g. an ethereal solvent such as 1,4-dioxane.

The intermediates of formula (VII) and (VIII) wherein L² represents a halogen atom, e.g. bromo or iodo, may be prepared by reacting a compound of formula (IX) or (X) respectively:

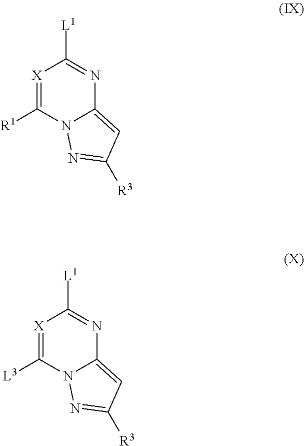

wherein X, R¹, R³, L¹ and L³ are as defined above; with a halogenating agent, e.g. elemental bromine or N-iodosuccinimide.

The intermediates of formula (IX) where X represents N, R¹ represents —NH₂ and L¹ represents methylsulfanyl may be prepared by reacting dimethyl N-cyanodithioimino-carbonate with a compound of formula (XI):

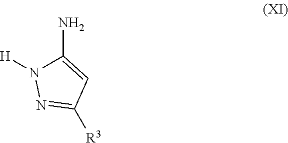

wherein R³ is as defined above; under conditions analogous to those described above for the reaction between dimethyl N-cyanodithioiminocarbonate and compound (V).

The intermediates of formula (III) wherein R¹ represents —NR^{b}R^{c} may be prepared by reacting a compound of formula H—NR^{b}R^{c} with a compound of formula (XII):

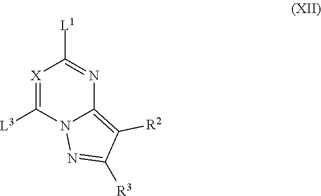

wherein X, R², R³, R^{b}, R^{c}, L¹ and L³ are as defined above; under conditions analogous to those described above for the reaction between a compound of formula H—NR^{b}R^{c} and compound (VIII).

The intermediates of formula (XII) wherein X represents CH and L¹ and L³ both represent chloro may be prepared by a two-step procedure which comprises: (i) reacting a compound of formula (V) as defined above with diethyl malonate; and (ii) treatment of the material thereby obtained with phosphoryl chloride.

Step (i) of the above procedure is conveniently effected at an elevated temperature in a suitable solvent, e.g. a lower alkanol such as ethanol. The reaction will typically be performed in the presence of a suitable base, e.g. an alkali metal alkoxide such as sodium ethoxide.

Step (ii) of the above procedure is conveniently effected at an elevated temperature in a suitable solvent, e.g. an aniline derivative such as N,N-diethylaniline.

As will be appreciated, the intermediates of formula (VI) above wherein L² represents halogen correspond to compounds in accordance with the present invention wherein R² represents halogen.

Where they are not commercially available, the starting materials of formula (IV), (V), (X) and (XI) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N-BOC moiety may be converted into the corresponding compound comprising a N—H moiety by treatment with an acid, e.g. a mineral acid such as hydrochloric acid, or an organic acid such as trifluoroacetic acid.

A compound wherein R¹ represents halogen, e.g. chloro, may be converted into the corresponding compound wherein R¹ represents amino (—NH₂) in a two-step procedure which comprises: (i) treatment with benzylamine; and (ii) removal of the benzyl moiety from the material thereby obtained by catalytic hydrogenation. As a general matter, any compound wherein R¹ represents —NH-benzyl may be converted into the corresponding compound wherein R¹ represents amino (—NH₂) by catalytic hydrogenation.

A compound wherein R¹ represents —SR^{a} may be converted into the corresponding compound wherein R¹ represents —SO₂R^{a} by treatment with an oxidising agent, typically 3-chloroperoxybenzoic acid (MCPBA).

A compound wherein R¹ represents —SO₂R^{a}, e.g. methylsulfonyl, may be converted into the corresponding compound wherein R¹ represents —OR^{a} by treatment with a sodium salt of formula NaOR^{a}. Similarly, a compound wherein R¹ represents —SO₂R^{a}, e.g. methylsulfonyl, may be converted into the corresponding compound wherein R¹ represents cyano by treatment with a cyanide salt, e.g. an alkali metal cyanide salt such as sodium cyanide. Likewise, a compound wherein R¹ represents —SO₂R^{a}, e.g. methylsulfonyl, may be converted into the corresponding compound wherein R¹ represents —NR^{b}R^{c} by treatment with an amine of formula H—NR^{b}R^{c}. By analogy, a compound wherein R¹ represents —SO₂R^{a}, e.g. methylsulfonyl, may be converted into the corresponding compound wherein R¹ represents —NH₂ by treatment with ammonium hydroxide.

A compound wherein R¹ represents —NR^{c}COR^{d} may be converted into the corresponding compound wherein R¹ represents —NHR^{c} by treatment with a base, typically an alkali metal carbonate such as potassium carbonate.

A compound containing an —NR^{b}R^{c} moiety, wherein R^{b} represents 4-methoxy-phenyl, may be converted into the corresponding compound wherein R^{b} represents hydrogen, by treatment with an acid, e.g. an organic acid such as trifluoroacetic acid.

A compound wherein R² represents —CO₂R^{d}, in which R^{d} is other than hydrogen, may be converted into the corresponding compound wherein R² represents carboxy (—CO₂H) by treatment with a base, typically an alkali metal hydroxide such as sodium hydroxide.

A compound wherein R² represents carboxy (—CO₂H) may be converted into the corresponding compound wherein R² represents —CONR^{b}R^{c} or —CON(OR^{a})R^{b} by treatment with the appropriate reagent of formula H—NR^{b}R^{c} or H—N(OR^{a})R^{b} respectively. The reaction may typically be performed in the presence of a coupling agent such as 1-(3-dimethylaminopropyl)-3-ethylcarbodiimide hydrochloride (EDC) and an additive such as 1-hydroxybenzotriazole hydrate (HOBT), optionally in the presence of a base, e.g. an organic base such as N,N-diisopropylethylamine. Alternatively, the reaction may be performed in the presence of a coupling agent such as O-(benzotriazol-1-yl)-N,N,N′,N′-tetramethyluronium tetrafluoroborate (TBTU) and a base, e.g. an organic base such as N,N-diisopropylethylamine.

A compound wherein R² represents carboxy (—CO₂H) may be converted into the corresponding compound wherein R² represents —CONH₂ by treatment with ammonium chloride, typically in the presence of a coupling agent such as EDC and an additive such as HOBT, suitably in the presence of a base, e.g. an organic base such as diisopropylamine or N,N-diisopropylethylamine. A compound wherein R² represents —CONH₂ may be converted into the corresponding compound wherein R² represents cyano (—CN) by treatment with phosphorus oxychloride. Alternatively, a compound wherein R² represents —CONH₂ may be converted into the corresponding compound wherein R² represents cyano in a two-step procedure which comprises: (i) treatment with cyanuric chloride; and (ii) treatment of the material thereby obtained with water.

A compound wherein R² represents carboxy (—CO₂H) may be converted into the corresponding compound wherein R² represents hydrogen by heating in the presence of a base, e.g. an organic amine such as triethylamine.

A compound wherein R² represents carboxy (—CO₂H) may be converted into the corresponding compound wherein R² represents hydroxymethyl (—CH₂OH) in a two-step procedure which comprises: (i) treatment with ethyl chloroformate and triethylamine; and (ii) treatment of the material thereby obtained with a reducing agent, typically an alkali metal borohydride such as sodium borohydride.

A compound wherein R² represents carboxy (—CO₂H) may be converted into the corresponding compound wherein R² represents hydroxy in a two-step procedure which comprises: (i) treatment with diphenyl phosphoryl azide; and (ii) treatment of the material thereby obtained with water.

A compound wherein R² represents carboxy (—CO₂H) may be converted into the corresponding compound wherein R² represents —NHCO₂R^{d}, wherein R^{d} is other than hydrogen, in a two-step procedure which comprises: (i) treatment with diphenyl phosphoryl azide; and (ii) treatment of the material thereby obtained with the appropriate reagent of formula R^{d}—OH.

A compound wherein R² represents carboxy (—CO₂H) may be converted into the corresponding compound wherein R² represents a 3-substituted 1,2,4-oxadiazol-5-yl moiety in a two-step procedure which comprises: (i) treatment with an appropriately-substituted N′-hydroxyamidine derivative, typically in the presence of a coupling agent such as O-(7-azabenzotriazol-1-yl)-N,N,N′,N′-tetramethyluronium hexafluorophosphate (HATU), suitably in the presence of a base, e.g. an organic base such as N,N-diisopropyl-ethylamine; and (ii) treatment of the material thereby obtained with a strong base, suitably a strong inorganic base, e.g. an alkali metal tert-butoxide such as potassium tert-butoxide.

A compound wherein R² represents 4,5-dihydrooxazol-2-yl may be prepared from the corresponding compound wherein R² represents —CONR^{b}R^{c}, in which R^{b} represents —CH₂CH₂OH and R^{c} represents hydrogen, by heating with a condensing agent such as N,N′-diisopropylcarbodiimide, typically in the presence of copper(II) trifluoromethane-sulfonate.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The following Examples illustrate the preparation of compounds according to the invention.

The compounds in accordance with this invention potently inhibit the activity of human PI4KIIIβ.

### PI4KIIIβ Enzyme Inhibition Assay

### Procedure A

Compounds were assayed utilizing reagents from Invitrogen and Promega. Compounds were screened in 1% DMSO (final) as 3-fold serial dilutions from a starting concentration of 20 μM. The 2.5× PI4Kβ reagent, the 2.5× PI Lipid Kinase Substrate/ATP mixture and the 5× compounds were prepared in 20 mM Tris pH 7.5, 0.5 mM EGTA, 2 mM DTT, 5 mM MgCl₂, 0.4% Triton. The final 25 μL Kinase Reaction consisted of: 4 nM PI4Kβ, 100 μM PI Lipid Kinase Substrate (both Invitrogen), and compound. The final ATP concentration in the assay was 10 μM. The detection reagents consisted of ADP-Glo™ Reagent and ADP-Glo™ Detect Reagent (Promega).

Briefly, compound was added to PI4Kβ followed by addition of ATP/PI Lipid Kinase Substrate mixture. The reaction mixture was incubated for 60 minutes at room temperature. The ADP-Glo™ Reagent was added and the plate was incubated for 40 minutes at room temperature, followed by addition of ADP-Glo™ Detect Reagent. The plate was incubated for a further 120 minutes and read on a Luminescence plate reader. The data was fitted with XLfit from IDBS using model number 205.

### Procedure B

Compounds were assayed using a PI4Kbeta Adapta assay. Compounds were screened in 1% DMSO (final) as 3-fold serial dilutions from a starting concentration of 10 μM. The 2× PI4KB (PI4K beta)/PI Lipid Kinase Substrate mixture was prepared in 50 mM HEPES pH 7.5, 0.1% CHAPS, 1 mM EGTA, 4 mM MgCl₂. The final 10 μL Kinase Reaction consisted of 7.5-60 ng PI4Kβ, and 100 μM PI Lipid Kinase Substrate in 32.5 mM HEPES pH 7.5, 0.05% CHAPS, 0.5 mM EGTA, 2 mM MgCl₂. The final ATP concentration in the assay was 10 μM. The detection mix consisted of EDTA (30 mM), Eu-anti-ADP antibody (6 nM) and ADP tracer. The detection mix contained the EC60 concentration of tracer for 5-150 μM ATP.

Briefly, ATP was added to compound, followed by addition of a PI4Kβ/PI Lipid Kinase Substrate mixture. The plate was shaken for 30 seconds to mix, then briefly centrifuged. The reaction mixture was incubated for 60 minutes at room temperature. The detection mix was added, then the plate was shaken and centrifuged. The plate was incubated for 60 minutes at room temperature and read on a fluorescence plate reader. The data was fitted with XLfit from IDBS using model number 205.

When tested in the above assay (Procedure A or Procedure B), the compounds of the accompanying Examples were all found to possess IC₅₀ values for inhibition of the activity of human PI4KIIIβ of 50 μM or better.

Certain compounds in accordance with this invention are potent inhibitors when measured in the MLR test described below.

### The Mixed Lymphocyte Reaction (MLR) Test

Human peripheral blood mononuclear cells (PBMCs) were isolated from buffy coats, obtained from healthy blood donors by Ficoll (Lymphoprep, Axis-Shield PoC AS, Oslo, Norway) density-gradient centrifugation. The cells at the Ficoll-plasma interface were washed three times and used as “Responder” cells. RPMI 1788 (ATCC, N° CCL-156) cells were treated with mitomycin C (Kyowa, Nycomed, Brussels, Belgium) and used as “Stimulator” cells. Responder cells (0.12×106), Stimulator cells (0.045×106) and compounds (in different concentrations) were cocultured for 6 days in RPMI 1640 medium (BioWhittaker, Lonza, Belgium) supplemented with 10% fetal calf serum, 100 U/ml Geneticin (Gibco, LifeTechnologies, UK). Cells were cultured in triplicate in flat-bottomed 96-well microtiter tissue culture plates (TTP, Switzerland). After 5 days, cells were pulsed with 1 μCi of methyl-³H thymidine (MP Biomedicals, USA), harvested 18 h later on glass filter paper and counted. Proliferation values were expressed as counts per minute (cpm), and converted to % inhibition with respect to a blank MLR test (identical but without added compound). The IC₅₀ was determined from a graph with at least four points, each derived from the mean of 2 experiments. The IC₅₀ value represents the lowest concentration of test compound (expressed in μM) that resulted in a 50% inhibition of the MLR.

Certain compounds of the accompanying Examples were found to generate IC₅₀ values in the MLR test of 10 μM or better.

### EXAMPLES

### Abbreviations

THF: tetrahydrofuran
MeOH: methanol
DMF: N,N-dimethylformamide
DMSO: dimethyl sulfoxide
DCM: dichloromethane
DIPEA: N,N-diisopropylethylamine
EtOAc: ethyl acetate
MCPBA: 3-chloroperoxybenzoic acid
TFA: trifluoroacetic acid
h: hour
r.t.: room temperature
MS: Mass Spectrometry
M: mass
LCMS: Liquid Chromatography Mass Spectrometry
HPLC: High Performance Liquid Chromatography
ES+: Electrospray Positive Ionisation
RT: retention time

### Analytical and Purification Methods

### Method 1

Column: Phenomenex Kinetex-XB C18 (2.1×100 mm, 1.7 μm column)
Flow rate: 0.6 mL/minute
Solvent A: 0.1% formic acid/water
Solvent B: 0.1% formic acid/acetonitrile
Injection volume: 3 μL
Column temperature: 40° C.
UV detection wavelength: 215 nm
Eluent: 0 to 5.3 minutes, constant gradient from 95% solvent A+5% solvent B to 100% solvent B; 5.3 to 5.8 minutes, 100% solvent B; 5.80 to 5.82 minutes, constant gradient from 100% solvent B to 95% solvent A+5% solvent B.
MS detection using Waters LCT or LCT Premier, or ZQ or ZMD
UV detection using Waters 2996 photodiode array or Waters 2787 UV or Waters 2788 UV

### Method 2

### High pH (Approximately pH 9.5)

Column: Waters XBridge, C18, 2.1×20 mm, 2.5 μm
Solvent A: 10 mM ammonium formate in water+0.1% ammonia solution
Solvent B: acetonitrile+5% solvent A+0.1% ammonia solution
Gradient Program:

| Time | A % | B % |
|---|---|---|
| 0.00 | 95.0 | 5.0 |
| 1.50 | 5.0 | 95.0 |
| 2.50 | 5.0 | 95.0 |
| 3.00 | 95.0 | 5.0 |

### Method 3

### High pH (Approximately pH 9.5)

Column: Waters XBridge, C18, 2.1×20 mm, 2.5 μm
Solvent A: 10 mM ammonium formate in water+0.1% ammonia solution
Solvent B: acetonitrile+5% solvent A+0.1% ammonia solution
Gradient Program:

| Time | A % | B % |
|---|---|---|
| 0.00 | 95.0 | 5.0 |
| 4.00 | 5.0 | 95.0 |
| 5.00 | 5.0 | 95.0 |
| 5.10 | 95.0 | 5.0 |

### Method 4

Column: Waters Acquity UPLC BEH C18 2.1×50 mm column, 1.7 μm silica particle.
Flow rate: 1.0 mL/minute
Solvent A: 10 mM ammonium formate in water+0.1% ammonia solution
Solvent B: acetonitrile+0.1% ammonia solution+5% solvent A
Injection volume: 1 μL
Column temperature: 40° C.
UV detection wavelength: 210 to 400 nm
Gradient Program

| Time | A % | B % |
|---|---|---|
| 0.00 | 95.0 | 5.0 |
| 0.10 | 5.0 | 95.0 |
| 1.35 | 5.0 | 95.0 |
| 1.40 | 95.0 | 95.0 |
| 1.60 | 95.0 | 5.0 |

### Method 5

Column: Kinetex Core-Shell, C18, 50×521 mm, 5 μm
Flow rate: 1.2 mL/minute
Solvent A: water+0.1% formic acid
Solvent B: acetonitrile+0.1% formic acid
Injection volume: 1 or 3 μL
Column temperature: 40° C.
UV detection wavelength: 215 nm

| Time | A % | B % |
|---|---|---|
| 0.00 | 95 | 5 |
| 1.20 | 0 | 100 |
| 1.30 | 0 | 100 |
| 1.31 | 95 | 5 |

MS detection using Scan Pos (Shimadzu): 100-1000

### Method 6

Column: Waters XBridge, C18, 2.1×20 mm, 2.5 μm
Flow rate: 1 mL/minute
Solvent A: pH 10 buffer, ammonium hydrogen carbonate
Solvent B: acetonitrile
Injection volume: 5 μL
Column temperature: 25° C.

| Time | A % | B % |
|---|---|---|
| 0 | 100 | 0 |
| 0.18 | 95 | 5 |
| 1.80 | 5 | 95 |
| 2.40 | 5 | 95 |
| 2.47 | 100 | 0 |
| 3.10 | 100 | 0 |

### Method 7

Column: Supelco Ascentis Express, 2.1×30 mm, 2.7 μm
Flow rate: 1 mL/minute
Solvent A: water+0.1% formic acid
Solvent B: acetonitrile+0.1% formic acid
Injection volume: 3 μL
Column temperature: 40° C.
UV detection wavelength: 215 nm

| Time | A % | B % |
|---|---|---|
| 0 | 95 | 5 |
| 1.50 | 0 | 100 |
| 1.60 | 0 | 100 |
| 1.61 | 95 | 5 |

### Method 8

Column: Waters X-Bridge, C18, 2.1×20 mm, 2.5 μm
Flow rate: 1 mL/minute
Solvent A: 10 mM ammonium formate in water+0.1% formic acid
Solvent B: acetonitrile+0.1% formic acid
Injection volume: 1-5 μL
Column temperature: 40° C.
UV detection wavelength: 210 to 400 nm

| Time | A % | B % |
|---|---|---|
| 0.00 | 95.0 | 5.0 |
| 1.50 | 5.0 | 95.0 |
| 2.25 | 5.0 | 95.0 |
| 2.30 | 95.0 | 5.0 |

### Method 9

### Low pH (Approximately pH 3)

Column: Waters XBridge, C18, 2.1×20 mm, 2.5 μm
Solvent A: water+0.1% formic acid
Solvent B: acetonitrile+5% solvent A+0.1% formic acid
Gradient Program:

| Time | A % | B % |
|---|---|---|
| 0.00 | 95.0 | 5.0 |
| 4.00 | 5.0 | 95.0 |
| 5.00 | 5.0 | 95.0 |
| 5.10 | 95.0 | 5.0 |

### Method 10

### High pH (Approximately pH 9.5)

Column: Waters Acquity UPLC BEH, C18, 2.1×50 mm, 1.7 μm
Solvent A: 10 mM ammonium formate in water+0.1% ammonia solution
Solvent B: acetonitrile+5% solvent A+0.1% ammonia solution
Gradient Program:

| Time | A % | B % |
|---|---|---|
| 0.00 | 98.0 | 2.0 |
| 4.00 | 5.0 | 95.0 |
| 5.00 | 5.0 | 95.0 |
| 5.10 | 98.0 | 2.0 |

### Method 11

### Low pH

Column: Waters Acquity UPLC HSS T3, C18, 2.1×100 mm, 1.7 μm
Flow rate: 0.4 mL/minute to 0.5 mL/minute
Solvent A: formic acid (0.5 mL/L) in acetonitrile/water (5:95)
Solvent B: formic acid (0.375 mL/L) in acetonitrile
Injection volume: 0.3 μL
Column temperature: 45° C.
UV detection wavelength: 210 to 400 nm
Gradient Program:

| | | | Flow rate |
|---|---|---|---|
| Time | A % | B % | (mL/minute) |
| 0.00 | 99 | 1 | 0.4 |
| 0.80 | 99 | 1 | 0.4 |
| 5.30 | 5 | 95 | 0.4 |
| 5.35 | 5 | 95 | 0.5 |
| 7.30 | 5 | 95 | 0.5 |
| 7.35 | 99 | 1 | 0.4 |
| 9.00 | 99 | 1 | 0.4 |

### Preparative HPLC

### Acidic Method

Flow rate: 40 mL/minute
Mobile Phase A: water with 0.1% formic acid
Mobile Phase B: acetonitrile with 0.1% formic acid
Column: Waters Sunfire, C18, 30 mm×100 mm
Particle Size: 10 μm
Runtime: 25.5 minutes
Inlet method: LC7_40 mL_7030_tubes.w60
Method Gradient: T=0 minutes, 75% A; 25% B
   T=2 minutes, 75% A; 25% B
   T=2.5 minutes, 70% A; 30% B
   T=18.5 minutes, 0% A; 100% B
   T=21.5 minutes, 0% A; 100% B
   T=22.5 minutes, 99% A; 1% B
   T=23.0 minutes, 99% A; 1% B
ACD Flow: 2 mL/minute (acetonitrile with 0.1% formic acid) throughout run
Primary wavelength (collection): 215 nm

### Basic Method

Flow rate: 40 mL/minute
Mobile Phase A: water+0.2% ammonium hydroxide
Mobile Phase B: acetonitrile+0.2% ammonium hydroxide
Column: Waters Sunfire, C18, 30 mm×100 mm
Particle Size: 5 μm
Runtime: 15.5 minutes
Method (isocratic): T=0 minutes, 95% A; 5% B
   T=2 minutes, 85% A; 15% B
   T=12.0 minutes, 70% A; 30% B
   T=12.5 minutes, 5% A; 95% B
   T=15.0 minutes, 5% A; 95% B
   T=15.5 minutes, 95% A; 5% B
Primary wavelength (collection): 215 nm
Secondary wavelength: 254 nm
Equipment: Gilson 215 Liquid Handler, 2× Gilson 306 Pumps, Gilson 805 Manometric Module, Gilson 119 UV/Vis Dual Detector.
Software: Gilson Unipoint V5.11

### Intermediate 1

### 3-Bromo-5,7-dichloro-2-methylpyrazolo[1,5-a]pyrimidine

To 5,7-dichloro-2-methylpyrazolo[1,5-a]pyrimidine (1.88 g, 9.28 mmol) in MeOH (25 mL) and water (25 mL), cooled to −2° C., was added bromine (574 μL, 11.14 mmol) over 3 minutes. The solution was stirred at between −5° C. and 0° C. for 30 minutes. The reaction mixture was filtered and washed with MeOH/water (1:1 mixture; 20 mL) at 0° C. to afford the title compound (1.60 g, 61%) as a light yellow solid. δ_{H} (CDCl₃) 6.95 (s, 1H), 2.55 (s, 3H). LCMS (ES+) [M+H]⁻ 281.95, RT 1.56 minutes (method 6).

### Intermediate 2

### 3-Bromo-5-chloro-N-[(1,3-dimethyl-1H-pyrazol-5-yl)methyl]-2-methylpyrazolo[1,5-a]-pyrimidin-7-amine

Intermediate 1 (2 g, 7.12 mmol) was dissolved in 2-propanol (20 mL), then 1-(1,3-dimethyl-1H-pyrazol-5-yl)methanamine (1.07 g, 8.54 mmol) and DIPEA (2.49 mL, 14.2 mmol) were added. The reaction mixture was stirred at 80° C. for 1 h. Upon cooling to r.t., the reaction mixture was evaporated to one quarter of its original volume. Heptane was added to the reaction mixture. The resulting white precipitate was filtered and dried in vacuo at 40° C. for 18 h to afford the title compound (3.54 g, 94%) as a cream solid, which was utilised without further purification. δ_{H} (DMSO-d₆, 250 MHz) 8.99 (t, J 6.3 Hz, 1H), 6.33 (s, 1H), 5.95 (s, 1H), 4.63 (d, J 6.2 Hz, 2H), 3.73 (s, 3H), 2.40 (s, 3H), 2.05 (s, 3H). LCMS (ES+) [M+H]⁻ 369/371, RT 1.11 minutes (method 5).

### Intermediate 3

### 3-Bromo-5-chloro-N-[(2,4-dimethyl-1,3-thiazol-5-yl)methyl]-2-methylpyrazolo[1,5-a]-pyrimidin-7-amine

Intermediate 1 (2 g, 7.12 mmol) was dissolved in 2-propanol (30 mL), then 1-(2,4-dimethyl-1,3-thiazol-5-yl)methanamine (1.01 g, 7.12 mmol) and DIPEA (2.50 mL, 14.2 mmol) were added. The reaction mixture was stirred at 80° C. for 4 h, then left to stand at r.t. overnight. The reaction mixture was concentrated in vacuo, then partitioned between EtOAc (100 mL) and saturated aqueous sodium hydrogen carbonate solution (150 mL). The aqueous layer was separated and extracted with EtOAc (2×100 mL). The combined organic layers were dried with anhydrous magnesium sulfate, then filtered and concentrated, to afford the title compound (2.73 g, 97%) as a yellow solid, which was utilised without further purification. δ_{H} (CDCl₃, 500 MHz) 6.48 (t, J 4.9 Hz, 1H), 5.98 (s, 1H), 4.62 (d, J 5.4 Hz, 2H), 2.66 (s, 3H), 2.44 (s, 3H), 2.42 (s, 3H). LCMS (ES+) [M+H]⁺ 386/388, RT 1.19 minutes (method 5).

### Intermediate 4

### 3-Bromo-5-chloro-N-{[3-(methanesulfonyl)phenyl]methyl}-2-methylpyrazolo[1,5-a]-pyrimidin-7-amine

Intermediate 1 (1 g, 3.56 mmol) was dissolved in 2-propanol (15 mL), then 1-[3-(methylsulfonyl)phenyl]methanamine (0.79 g, 4.28 mmol) and DIPEA (1.24 mL, 7.12 mmol) were added. The reaction mixture was stirred at 80° C. for 4 h, then left to stand at r.t. overnight. The reaction mixture was concentrated in vacuo, then partitioned between DCM (50 mL) and saturated aqueous sodium hydrogen carbonate solution (75 mL). The aqueous layer was separated and extracted with DCM (2×50 mL). The combined organic layers were dried with anhydrous magnesium sulfate, then filtered and concentrated, to afford the title compound (1.41 g, 88%) as a yellow solid, which was utilised without further purification. δ_{H} (DMSO-d₆, 500 MHz) 9.20 (t, J 6.5 Hz, 1H), 8.01 (s, 1H), 7.84 (d, J 7.9 Hz, 1H), 7.75 (d, J 7.8 Hz, 1H), 7.63 (t, J 7.8 Hz, 1H), 6.33 (s, 1H), 4.75 (d, J 6.0 Hz, 2H), 3.20 (s, 3H), 2.41 (s, 3H). LCMS (ES+) [M+H]⁺ 429/431, RT 1.15 minutes (method 5).

### Intermediate 5

### 3-Bromo-5-chloro-2-methyl-N-[(5-methylisoxazol-3-yl)methyl]pyrazolo[1,5-a]pyrimidin-7-amine

To a stirred solution of Intermediate 1 (4.51 g, 16.1 mmol) in 2-propanol (5 mL) was added DIPEA (4.61 g, 35.7 mmol), followed by (5-methylisoxazol-3-yl)methylamine (2 g, 17.8 mmol). The reaction mixture was heated at 80° C. for 3 h. The resulting precipitate was filtered to give a pure white solid (4.1 g). A second crop was obtained from the mother liquors and combined with the first crop to afford the title compound (6.16 g, 96.9%) as a white solid. LCMS (ES+) [M+H]⁺ 356.0/358.0, RT 1.43 minutes (method 2).

### Intermediate 6

### 3-Bromo-5-chloro-2-methylpyrazolo[1,5-a]pyrimidin-7-amine

Intermediate 1 (1 g, 3.56 mmol) in aqueous ammonia (35%, 10 mL, 87.9 mmol) and 1,4-dioxane (10 mL) was heated in two 20 mL pressure tubes (10 mL in each) at 85° C. with stirring for 2 h. The reaction mixtures were combined and diluted with water. The precipitate was collected by filtration to afford the title compound (930 mg, quantitative) as a yellow solid. δ_{H} (DMSO-d₆, 250 MHz) 8.21 (s, 2H), 6.05 (s, 1H), 2.38 (s, 3H).

### Intermediate 7

### tert-Butyl N-(3-bromo-5-chloro-2-methylpyrazolo[1,5-a]pyrimidin-7-yl)-N-[(1,3-dimethyl-1H-pyrazol-5-yl)methyl]carbamate

To a stirred solution of Intermediate 2 (1 eq) in 1,4-dioxane was added di-tert-butyl dicarbonate (2 eq), followed by 4-(dimethylamino)pyridine (0.1 eq). The reaction mixture was stirred at r.t. for 4-18 h, then concentrated in vacuo and purified by flash column chromatography (gradient elution with 0-100% EtOAc/heptane) to afford the title compound. δ_{H} (CDCl₃, 500 MHz) 6.50 (s, 1H), 5.70 (s, 1H), 5.05 (s, 2H), 3.79 (s, 3H), 2.50 (s, 3H), 2.14 (s, 3H), 1.38 (s, 9H). LCMS (ES+) [M+H]⁺ 469/471, RT 1.34 minutes (method 5).

### Intermediate 8

### tert-Butyl N-(3-bromo-5-chloro-2-methylpyrazolo[1,5-a]pyrimidin-7-yl)-N-[(2,4-dimethyl-1,3-thiazol-5-yl)methyl]carbamate

Prepared from Intermediate 3 according to the method described for Intermediate 7. δ_{H} (CDCl₃, 500 MHz) 6.54 (s, 1H), 5.19 (s, 2H), 2.60 (s, 3H), 2.52 (s, 3H), 2.05 (s, 3H), 1.44 (s, 9H). LCMS (ES+) [M+H]⁻ 486/488, RT 1.40 minutes (method 5).

### Intermediate 9

### tert-butyl N-(3-Bromo-5-chloro-2-methylpyrazolo[1,5-a]pyrimidin-7-yl)-N-{[3-(methanesulfonyl)phenyl]methyl}carbamate

Prepared from Intermediate 4 according to the method described for Intermediate 7. δ_{H} (CDCl₃, 500 MHz) 7.97-7.90 (m, 1H), 7.84 (dt, J 7.6, 1.4 Hz, 1H), 7.55 (d, J 7.8 Hz, 1H), 7.50 (t, J 7.7 Hz, 1H), 6.58 (s, 1H), 5.10 (s, 2H), 2.98 (s, 3H), 2.52 (s, 3H), 1.39 (s, 9H). LCMS (ES+) [M+H]⁻ 529/531, RT 1.35 minutes (method 5).

### Intermediate 10

### tert-Butyl N-[3-bromo-5-(3,3-difluoroazetidin-1-yl)-2-methylpyrazolo[1,5-a]pyrimidin-7-yl]-N-[(1,3-dimethyl-1H-pyrazol-5-yl)methyl]carbamate

To Intermediate 7 (1 eq) were added 3,3-difluoroazetidine (2 eq), DIPEA (3.5 eq) and acetonitrile (5-20 mL). The reaction mixture was heated at 90° C. for 18 h in a sealed tube, then cooled to r.t. and concentrated in vacuo. Purification by flash column chromatography on silica (gradient elution with 0-100% EtOAc/heptane) afforded the title compound. δ_{H} (CDCl₃, 500 MHz) 5.77 (s, 1H), 5.55 (s, 1H), 4.99 (s, 2H), 4.43 (t, J 11.8 Hz, 4H), 3.79 (s, 3H), 2.41 (s, 3H), 2.19 (s, 3H), 1.38 (s, 9H). LCMS (ES+) [M+H]⁺ 526/528, RT 1.31 minutes (method 5).

### Intermediate 11

### tert-Butyl N-[3-bromo-2-methyl-5-(2-oxa-6-azaspiro[3.3 ]heptan-6-yl)pyrazolo[1,5-a]-pyrimidin-7-yl]-N-[(1,3-dimethyl-1H-pyrazol-5-yl)methyl]carbamate

Prepared from Intermediate 7 and 2-oxa-6-azaspiro[3.3]heptane according to the method described for Intermediate 10. δ_{H} (CDCl₃, 500 MHz) 5.78 (s, 1H), 5.47 (s, 1H), 4.98 (s, 2H), 4.84 (s, 4H), 4.25 (s, 4H), 3.76 (s, 3H), 2.38 (s, 3H), 2.20 (s, 3H), 1.37 (s, 9H). LCMS (ES+) [M+H]⁺ 532/534, RT 1.20 minutes (method 5).

### Intermediate 12

### tert-Butyl N-[5-(4-acetylpiperazin-1-yl)-3-bromo-2-methylpyrazolo[1,5-a]pyrimidin-7-yl]-N-[(1,3-dimethyl-1H-pyrazol-5-yl)methyl]carbamate

Prepared from Intermediate 7 and 1-acetylpiperazine according to the method described for Intermediate 10. δ_{H} (CDCl₃, 500 MHz) 5.82 (s, 1H), 5.79 (s, 1H), 4.99 (s, 2H), 3.76 (s, 3H), 3.75-3.69 (m, 4H), 3.63-3.51 (m, 4H), 2.40 (s, 3H), 2.18 (s, 3H), 2.15 (s, 3H), 1.38 (s, 9H). LCMS (ES+) [M+H]⁺ 561/563, RT 1.19 minutes (method 5).

### Intermediate 13

### tert-Butyl N-[5-(4-acetylpiperazin-1-yl)-3-bromo-2-methylpyrazolo[1,5-a]pyrimidin-7-yl]-N-[(2,4-dimethyl-1,3-thiazol-5-yl)methyl]carbamate

Prepared from Intermediate 8 and 1-acetylpiperazine according to the method described for Intermediate 10. δ_{H} (DMSO-d₆, 500 MHz) 6.64 (s, 1H), 5.03 (s, 2H), 3.75-3.69 (m, 2H), 3.66-3.60 (m, 2H), 3.58-3.51 (m, 4H), 2.27 (s, 3H), 2.08 (s, 3H), 2.05 (s, 3H), 1.31 (s, 9H), 1.29-1.22 (m, 3H). LCMS (ES+) [M+H]⁺ 578/580, RT 1.26 minutes (method 5).

### Intermediate 14

### tert-Butyl N-[5-(4-acetylpiperazin-1-yl)-3-bromo-2-methylpyrazolo[1,5-a]pyrimidin-7-yl]-N-{[3-(methanesulfonyl)phenyl]methyl}carbamate

Prepared from Intermediate 9 and 1-acetylpiperazine according to the method described for Intermediate 10. δ_{H} (DMSO-d₆, 500 MHz) 8.04-7.97 (m, 1H), 7.81-7.75 (m, 1H), 7.73-7.65 (m, 1H), 7.56 (t, J 7.7 Hz, 1H), 6.76 (s, 1H), 5.04 (s, 2H), 3.77-3.67 (m, 2H), 3.65-3.59 (m, 2H), 3.57-3.49 (m, 4H), 3.10 (s, 3H), 2.30 (s, 3H), 2.04 (s, 3H), 1.29 (s, 9H). LCMS (ES+) [M+H]⁺ 621/623, RT 1.25 minutes (method 5).

### Intermediate 15

### Ethyl 4-[3-bromo-7-(N-[(tert-butoxy)carbonyl]-N-{[3-(methanesulfonyl)phenyl]methyl}-amino)-2-methylpyrazolo[1,5-a]pyrimidin-5-yl]piperazine-1-carboxylate

Prepared from Intermediate 9 and ethyl piperazine-1-carboxylate according to the method described for Intermediate 10. δ_{H} (DMSO-d₆, 500 MHz) 8.04-7.98 (m, 1H), 7.83-7.75 (m, 1H), 7.72-7.66 (m, 1H), 7.56 (t, J 7.7 Hz, 1H), 6.74 (s, 1H), 5.03 (s, 2H), 4.08 (q, J 7.1 Hz, 2H), 3.69-3.62 (m, 4H), 3.52-3.42 (m, 4H), 3.10 (s, 3H), 2.30 (s, 3H), 1.29 (s, 9H), 1.20 (t, J 7.1 Hz, 3H). LCMS (ES+) [M+H] 651/653, RT 1.42 minutes (method 5).

### Intermediate 16

### 3-Bromo-2-methyl-N-[(5-methylisoxazol-3-yl)methyl]-5-(morpholin-4-yl)pyrazolo[1,5-a]pyrimidin-7-amine

A microwave vial was charged with Intermediate 5 (0.6 g, 1.68 mmol), followed by morpholine (2.01 g, 22.9 mmol) and 2-propanol (2 mL). The reaction mixture was heated at 140° C. for 2 h. Upon cooling, the solid was filtered and dried to afford the title compound (0.41 g, 60%). LCMS (ES+) [M+H]⁺ 409.2, RT 1.43 minutes (method 2).

### Intermediate 17

### Ethyl 4-(3-bromo-7-{[(tert-butoxy)carbonyl]amino}-2-methylpyrazolo[1,5-a]pyrimidin-5-yl)piperazine-1-carboxylate

Intermediate 6 (930 mg, 3.56 mmol) was stirred in DCM (50 mL). Di-tert-butyl dicarbonate (1.94 g, 8.89 mmol) was added, followed by 4-(dimethylamino)pyridine (40 mg). The reaction mixture was stirred at r.t. for 18 h. Imidazole (0.48 g, 7.11 mmol) was added and the reaction mixture was stirred for 30 minutes to remove excess di-tert-butyl dicarbonate. The reaction mixture was diluted with DCM (50 mL) and washed sequentially with 0.5M aqueous hydrochloric acid (2×50 mL), followed by saturated aqueous sodium chloride solution (30 mL). The organic phase was dried with anhydrous sodium sulfate and concentrated in vacuo. The resulting mixture was treated with ethyl piperazine-1-carboxylate (1.13 g, 7.11 mmol) and DIPEA (2.17 mL, 12.45 mmol), then heated in acetonitrile (10 mL) in a 20 mL sealed pressure tube at 90° C. with stirring for 18 h. The reaction mixture was diluted with water (50 mL) and extracted with DCM (2×50 mL). The organic phase was dried with anhydrous sodium sulfate and concentrated in vacuo. The resulting yellow solid was purified by flash column chromatography on silica (gradient elution with 0-100% EtOAc/heptane), then the relevant fractions were concentrated in vacuo. The resulting yellow solid (1.3 g) was triturated with 30% methyl tert-butyl ether in heptane and collected by filtration to afford a white solid (410 mg). The filtrate was concentrated in vacuo and the solid obtained was triturated using heptane, then collected by filtration, to afford further white solid (368 mg). The filtrate was combined with impure column chromatography fractions and concentrated in vacuo. The residue was purified by flash column chromatography on silica (gradient elution with 0-100% EtOAc/heptane). The resulting sticky white solid was triturated with heptane and collected by filtration. The resulting material (570 mg) was combined with the previous batches to afford the title compound (1.35 g, 78%) as a white solid. δ_{H} (DMSO-d₆, 250 MHz) 9.31 (s, 1H), 6.90 (s, 1H), 4.08 (q, J7.1 Hz, 2H), 3.74-3.59 (m, 4H), 3.59-3.40 (m, 4H), 2.30 (s, 3H), 1.51 (s, 9H), 1.21 (t, J 7.1 Hz, 3H). LCMS (ES+) [M+H]⁺ 483.2/485.2, RT 2.21 minutes (method 8).

### Intermediate 18

### 5-Bromo-1,3-dimethyl-1H-indazole

To a stirred solution of 5-bromo-3-methyl-1H-indazole (2.51 g, 11.6 mmol), dissolved in N,N-dimethylformamide (30 mL) and cooled to 0° C. under nitrogen, was added portionwise sodium hydride (60% dispersion in mineral oil; 596 mg, 14.9 mmol). The dark brown, effervescing solution was stirred for 70 minutes prior to addition of iodomethane (0.87 mL, 14 mmol). The reaction mixture was stirred at 0° C. for 15 minutes before warming to r.t. A brown-orange solid was formed and the mixture was stirred for 3 h prior to the addition of water (30 mL) and EtOAc (30 mL). The mixture was stirred for 40 minutes before leaving to stand overnight. Further EtOAc (20 mL) and water (20 mL) were added, then the organic layer was separated. The aqueous layer was re-extracted with further EtOAc (2×50 mL). The organic layers were combined, dried with anhydrous sodium sulfate and filtered under reduced pressure, then the solvent was removed in vacuo. The resulting brown oil was purified by flash column chromatography on silica (gradient elution with 0-100% EtOAc/isohexane) to afford the title compound (1.75 g, 67%) as an orange oil. δ_{H} (DMSO-d₆, 300 MHz) 7.94 (dd, J 1.7, 0.7 Hz, 1H), 7.55 (dd, J 8.8, 0.7 Hz, 1H), 7.46 (dd, J 8.9, 1.8 Hz, 1H), 3.95 (s, 3H), 2.45 (s, 3H). LCMS (ES+) [M+H]⁺ 227.0, RT 2.00 minutes (method 3).

### Intermediate 19

### 1,3-Dimethyl-5-(4,4,5,5-tetramethyl-1,3,2-dioxaborolan-2-yl)-1H-indazole

To a solution of Intermediate 18 (1.43 g, 6.35 mmol) in 1,4-dioxane (15 mL) were added bis(pinacolato)diboron (1.77 g, 6.99 mmol) and potassium acetate (1.25 g, 12.71 mmol), then the system was degassed under nitrogen for 30 minutes. [1,1′-Bis(diphenyl-phosphino)ferrocene]dichloropalladium(II) complex with DCM (0.26 g, 0.32 mmol) was added and the mixture was heated at 80° C. for 16 h. Upon cooling to r.t., the reaction mixture was diluted with EtOAc and filtered through a pad of Celite which was washed with additional EtOAc, then the combined filtrates were concentrated in vacuo. The residue was purified by flash column chromatography on silica (gradient elution with 0-100% EtOAc in heptane) to afford the title compound (1.61 g, 91%) as a white solid. δ_{H} (DMSO-d₆, 500 MHz) 8.09-8.01 (m, 1H), 7.62 (dd, J 8.5, 0.9 Hz, 1H), 7.52 (dd, J 8.5, 0.8 Hz, 1H), 3.95 (s, 3H), 2.49 (s, 3H), 1.31 (s, 12H). LCMS (ES+) [M+H]⁺ 273, RT 1.25 minutes (method 5).

### Intermediate 20

### 5-Bromo-2-chloro-N-methylbenzamide

5-Bromo-2-chlorobenzoic acid (8 g, 34 mmol) was stirred in DCM (150 mL) as a suspension and cooled in an ice bath. DMF (0.1 mL, catalytic) was added, followed by oxalyl chloride (3.5 mL, 41 mmol) dropwise. The reaction mixture was allowed to warm to r.t. and was stirred for 2 h, then concentrated in vacuo. THF (50 mL) was added and the reaction mixture was cooled to 0° C. A mixture of methylamine in THF (2M; 25.5 mL) and DIPEA (8.9 mL, 50.1 mmol) was rapidly added. The suspension was stirred at r.t. for 20 minutes. The reaction mixture was diluted with EtOAc (150 mL), then washed with 1M aqueous hydrochloric acid (50 mL), saturated aqueous sodium hydrogen carbonate solution (50 mL) and saturated aqueous sodium chloride solution (30 mL). The organic phase was dried over anhydrous sodium sulfate and concentrated in vacuo to afford the title compound (8.41 g, 100%) as a white solid. δ_{H} (DMSO-d₆, 250 MHz) 8.49-8.35 (m, 1H), 7.72-7.58 (m, 2H), 7.53-7.39 (m, 1H), 2.75 (d, J 4.7 Hz, 3H).

### Intermediate 21

### 2-Chloro-N-methyl-5-(4,4,5,5-tetramethyl-1,3,2-dioxaborolan-2-yl)benzamide

To Intermediate 20 (8.41 g, 33.8 mmol) in 1,4-dioxane (120 mL) were added bis(pinacolato)diboron (9.45 g, 37.2 mmol) and potassium acetate (6.64 g, 67.7 mmol). The reaction mixture was purged with nitrogen for 5 minutes, then [1,1′-bis(diphenyl-phosphino)ferrocene]dichloropalladium(II) complex with DCM (1.38 g, 1.69 mmol) was added. The reaction mixture was heated at 80° C. with stirring for 2 h. The reaction mixture was allowed to cool and filtered through kieselguhr, then washed with EtOAc and concentrated in vacuo. The reaction was repeated using Intermediate 20 (7.75 g, 31.2 mmol) as before. The two batches were combined and purified by dry flash chromatography on silica (gradient elution with 0-70% EtOAc/heptane) to afford the title compound (21.9 g), which was utilised without further purification. δ_{H} (DMSO-d₆, 500 MHz) 8.39-8.30 (m, 1H), 7.67 (dd, J 8.0, 1.6 Hz, 1H), 7.63 (d, J 1.5 Hz, 1H), 7.50 (d, J 8.0 Hz, 1H), 2.75 (d, J 4.6 Hz, 3H), 1.30 (s, 12H).

### Intermediate 22

### tert-Butyl N-[5-(3,3-difluoroazetidin-1-yl)-3-(3,4-dimethoxyphenyl)-2-methylpyrazolo-[1,5-a]pyrimidin-7-yl]-N-[(1,3-dimethyl-1H-pyrazol-5-yl)methyl]carbamate

A mixture of Intermediate 10 (1 eq), tripotassium phosphate (3 eq) and (3,4-dimethoxyphenyl)boronic acid (1.5 eq) in 1,4-dioxane (6 mL) and water (1.5 mL) was placed in a 20 mL pressure tube. The reaction mixture was purged with nitrogen gas for 10 minutes. Tetrakis(triphenylphosphine) palladium(0) (0.04 eq) was added, then the reaction mixture was purged with nitrogen gas, sealed and heated at 100° C. with stirring for 4 h. Upon cooling to r.t., the reaction mixture was concentrated in vacuo and the residue was partitioned between water (20 mL) and EtOAc (10 mL). The aqueous layer was washed with further EtOAc (2×10 mL) and the organic layers were combined. The combined organic phase was washed with saturated aqueous sodium chloride solution (20 mL) and dried with anhydrous magnesium sulfate, then filtered and concentrated in vacuo. The residue was dried at 40° C. for 18 h to afford the title compound. LCMS (ES+) [M+H]⁻ 584, RT 1.31 minutes (method 5).

### Intermediate 23

### tert-Butyl N-{5-(3,3-difluoroazetidin-1-yl)-3-[3-(methanesulfonyl)-4-methoxyphenyl]-2-methylpyrazolo[1,5-a]pyrimidin-7-yl]-N-[(1,3-dimethyl-1H-pyrazol-5-yl)methyl]-carbamate

Prepared from Intermediate 10 and 2-[3-(methanesulfonyl)-4-methoxyphenyl]-4,4,5,5-tetramethyl-1,3,2-dioxaborolane according to the method described for Intermediate 22. LCMS (ES+) [M+H]⁺ 632, RT 1.31 minutes (method 5).

### Intermediate 24

### tert-Butyl N-[5-(3,3-difluoroazetidin-1-yl)-3-(1,3-dimethyl-1H-indazol-5-yl)-2-methylpyrazolo[1,5-a]pyrimidin-7-yl]-N-[(1,3-dimethyl-1H-pyrazol-5-yl)methyl]-carbamate

Prepared from Intermediate 10 and Intermediate 19 according to the method described for Intermediate 22. LCMS (ES+) [M+H]⁺ 592, RT 1.33 minutes (method 5).

### Intermediate 25

### tert-Butyl N-{5-(3,3-difluoroazetidin-1-yl)-3-[3-(difluoromethoxy)-4-methoxyphenyl]-2-methylpyrazolo[1,5-a]pyrimidin-7-yl}-N-[(1,3-dimethyl-1H-pyrazol-5-yl)methyl]-carbamate

Prepared from Intermediate 10 and 2-[3-(difluoromethoxy)-4-methoxyphenyl]-4,4,5,5-tetramethyl-1,3,2-dioxaborolane according to the method described for Intermediate 22. LCMS (ES+) [M+H]⁺ 620, RT 1.38 minutes (method 5).

### Intermediate 26

### tert-Butyl N-[3-(3,4-dimethoxyphenyl)-2-methyl-5-(2-oxa-6-azaspiro[3.3]heptan-6-yl)-pyrazolo[1,5-a]pyrimidin-7-yl]-N-[(1,3-dimethyl-1H-pyrazol-5-yl)methyl]carbamate

A mixture of Intermediate 11 (1 eq), tripotassium phosphate (3 eq) and (3,4-dimethoxyphenyl)boronic acid (1.5 eq) in 1,4-dioxane (4 mL) and water (0.65 mL) was placed in a 20 mL pressure tube. The reaction mixture was purged with nitrogen gas for 10 minutes. Tetrakis(triphenylphosphine) palladium(0) (0.05 eq) was added, then the reaction mixture was purged with nitrogen gas, sealed and heated at 100° C. with stirring for 5 h. Upon cooling to r.t., the reaction mixture was concentrated in vacuo and the residue was partitioned between water (20 mL) and EtOAc (20 mL). The aqueous layer was washed with further EtOAc (2×10 mL) and the organic layers were combined. The combined organic phase was washed with saturated aqueous sodium chloride solution (20 mL) and dried with anhydrous magnesium sulfate, then filtered and concentrated in vacuo. The residue was dried at 40° C. for 18 h to afford the title compound. LCMS (ES+) [M+H]⁺ 590, RT 1.22 minutes (method 5).

### Intermediate 27

### tert-Butyl N-[(1,3-dimethyl-1H-pyrazol-5-yl)methyl]-N-{3-[3-(methanesulfonyl)-4-methoxyphenyl]-2-methyl-5-(2-oxa-6-azaspiro[3.3]heptan-6-yl)pyrazolo[1,5-a]-pyrimidin-7-yl}carbamate

Prepared from Intermediate 11 and 2-[3-(methanesulfonyl)-4-methoxyphenyl]-4,4,5,5-tetramethyl-1,3,2-dioxaborolane according to the method described for Intermediate 26. LCMS (ES+) [M+H]⁺ 638, RT 1.15 minutes (method 5).

### Intermediate 28

### tert-Butyl N-[3-(1,3-dimethyl-1H-indazol-5-yl)-2-methyl-5-(2-oxa-6-azaspiro[3.3]heptan-6-yl)pyrazolo[1,5-a]pyrimidin-7-yl]-N-[(1,3-dimethyl-1H-pyrazol-5-yl)methyl]carbamate

Prepared from Intermediate 11 and Intermediate 19 according to the method described for Intermediate 26. LCMS (ES+) [M+H]⁺ 598, RT 1.23 minutes (method 5).

### Intermediate 29

### tert-Butyl N-{3-[3-(difluoromethoxy)-4-methoxyphenyl]-2-methyl-5-(2-oxa-6-azaspiro-[3.3]heptan-6-yl)pyrazolo[1,5-a]pyrimidin-7-yl}-N-[(1,3-dimethyl-1H-pyrazol-5-yl)-methyl]carbamate

Prepared from Intermediate 11 and 2-[3-(difluoromethoxy)-4-methoxyphenyl]-4,4,5,5-tetramethyl-1,3,2-dioxaborolane according to the method described for Intermediate 26. LCMS (ES+) [M+H]⁺ 626, RT 1.30 minutes (method 5).

### Intermediate 30

### tert-Butyl N-[5-(4-acetylpiperazin-1-yl)-3-(3,4-dimethoxyphenyl)-2-methylpyrazolo[1,5-a]pyrimidin-7-yl]-N-[(1,3-dimethyl-1H-pyrazol-5-yl)methyl]carbamate

A mixture of Intermediate 12 (1 eq), tripotassium phosphate (2.9 eq) and (3,4-dimethoxyphenyl)boronic acid (1.43 eq) in 1,4-dioxane (4 mL) and water (0.6 mL) was placed in a 20 mL pressure tube. The reaction mixture was purged with nitrogen gas for 10 minutes. Tetrakis(triphenylphosphine) palladium(0) (0.05 eq) was added, then the reaction mixture was purged with nitrogen gas, sealed and heated at 100° C. with stirring for 5 h. Upon cooling to r.t., the reaction mixture was diluted with EtOAc (40 mL) and dried with anhydrous magnesium sulfate, then filtered and concentrated in vacuo. The residue was dried at 40° C. for 18 h to afford the title compound. LCMS (ES+) [M+H]⁺ 619, RT 1.21 minutes (method 5).

### Intermediate 31

### tert-Butyl N-{5-(4-acetylpiperazin-1-yl)-3-[3-(methanesulfonyl)-4-methoxyphenyl]-2-methylpyrazolo[1,5-a]pyrimidin-7-yl}-N-[(1,3-dimethyl-1H-pyrazol-5-yl)methyl]-carbamate

Prepared from Intermediate 12 and 2-[3-(methanesulfonyl)-4-methoxyphenyl]-4,4,5,5-tetramethyl-1,3,2-dioxaborolane according to the method described for Intermediate 30. LCMS (ES+) [M+H]^{|} 667, RT 1.16 minutes (method 5).

### Intermediate 32

### tert-Butyl N-[5-(4-acetylpiperazin-1-yl)-3-(1,3-dimethyl-1H-indazol-5-yl)-2-methyl-pyrazolo[1,5-a]pyrimidin-7-yl]-N-[(1,3-dimethyl-1H-pyrazol-5-yl)methyl]carbamate

Prepared from Intermediate 12 and Intermediate 19 according to the method described for Intermediate 30. LCMS (ES+) [M+H]⁺ 627, RT 1.23 minutes (method 5).

### Intermediate 33

### tert-Butyl N-{5-(4-acetylpiperazin-1-yl)-3-[3-(difluoromethoxy)-4-methoxyphenyl]-2-methylpyrazolo[1,5-a]pyrimidin-7-yl}-N-[(1,3-dimethyl-1H-pyrazol-5-yl)methyl]-carbamate

Prepared from Intermediate 12 and 2-[3-(difluoromethoxy)-4-methoxyphenyl]-4,4,5,5-tetramethyl-1,3,2-dioxaborolane according to the method described for Intermediate 30. LCMS (ES+) [M+H]⁺ 655, RT 1.29 minutes (method 5).

### Intermediate 34

### tert-Butyl N-[5-(4-acetylpiperazin-1-yl)-3-(3,4-dimethoxyphenyl)-2-methylpyrazolo[1,5-a]pyrimidin-7-yl]-N-[(2,4-dimethyl-1,3-thiazol-5-yl)methyl]carbamate

A mixture of Intermediate 13 (1 eq), tripotassium phosphate (3 eq) and (3,4-dimethoxyphenyl)boronic acid (1.5 eq) in 1,4-dioxane (6 mL) and water (0.6 mL) was placed in a 20 mL pressure tube. The reaction mixture was purged with nitrogen gas for 10 minutes. Tetrakis(triphenylphosphine)palladium(0) (0.05 eq) was added, then the reaction mixture was purged with nitrogen gas, sealed and heated at 100° C. with stirring for 5 h. Upon cooling to r.t., the reaction mixture was diluted with EtOAc (40 mL) and dried with anhydrous magnesium sulfate, then filtered and concentrated in vacuo, to afford the title compound. LCMS (ES+) [M+H]⁺ 636, RT 1.25 minutes (method 5).

### Intermediate 35

### tert-Butyl N-{5-(4-acetylpiperazin-1-yl)-3-[3-(methanesulfonyl)-4-methoxyphenyl]-2-methylpyrazolo[1,5-a]pyrimidin-7-yl}-N-[(2,4-dimethyl-1,3-thiazol-5-yl)methyl]-carbamate

Prepared from Intermediate 13 and 2-[3-(methanesulfonyl)-4-methoxyphenyl]-4,4,5,5-tetramethyl-1,3,2-dioxaborolane according to the method described for Intermediate 34. LCMS (ES+) [M+H]⁺ 684, RT 1.19 minutes (method 5).

### Intermediate 36

### tert-Butyl N-{5-(4-acetylpiperazin-1-yl)-3-[3-(difluoromethoxy)-4-methoxyphenyl]-2-methylpyrazolo[1,5-a]pyrimidin-7-yl}-N-[(2,4-dimethyl-1,3-thiazol-5-yl)methyl]-carbamate

Prepared from Intermediate 13 and 2-[3-(difluoromethoxy)-4-methoxyphenyl]-4,4,5,5-tetramethyl-1,3,2-dioxaborolane according to the method described for Intermediate 34. LCMS (ES+) [M+H]⁺ 672, RT 1.33 minutes (method 5).

### Intermediate 37

### tert-Butyl N-[5-(4-acetylpiperazin-1-yl)-3-(3,4-dimethoxyphenyl)-2-methylpyrazolo[1,5-a]pyrimidin-7-yl]-N-{[3-methanesulfonyl)phenyl]methyl}carbamate

A mixture of Intermediate 14 (1 eq), tripotassium phosphate (2.93 eq) and (3,4-dimethoxyphenyl)boronic acid (1.5 eq) in 1,4-dioxane (4 mL) and water (0.59 mL) was placed in a 20 mL pressure tube. The reaction mixture was purged with nitrogen gas for 10 minutes. Tetrakis(triphenylphosphine)palladium(0) (0.05 eq) was added, then the reaction mixture was purged with nitrogen gas, sealed and heated at 100° C. with stirring for 5 h. Upon cooling to r.t., the reaction mixture was diluted with EtOAc (40 mL) and dried with anhydrous magnesium sulfate, then filtered and concentrated in vacuo. The residue was dried at 40° C. for 18 h to afford the title compound. LCMS (ES+) [M+H]⁺ 679, RT 1.21 minutes (method 5).

### Intermediate 38

### tert-Butyl N-{5-(4-acetylpiperazin-1-yl)-3-[3-(methanesulfonyl)-4-methoxyphenyl]-2-methylpyrazolo[1,5-a]pyrimidin-7-yl}-N-{[3-(methanesulfonyl)phenyl]methyl}carbamate

Prepared from Intermediate 14 and 2-[3-(methanesulfonyl)-4-methoxyphenyl]-4,4,5,5-tetramethyl-1,3,2-dioxaborolane according to the method described for Intermediate 37. LCMS (ES+) [M+H]⁺ 727, RT 1.16 minutes (method 5).

### Intermediate 39

### tert-Butyl N-{5-(4-acetylpiperazin-1-yl)-3-[3-(difluoromethoxy)-4-methoxyphenyl]-2-methylpyrazolo[1,5-a]pyrimidin-7-yl}-N-{[3-(methanesulfonyl)phenyl]methyl}carbamate

Prepared from Intermediate 14 and 2-[3-(difluoromethoxy)-4-methoxyphenyl]-4,4,5,5-tetramethyl-1,3,2-dioxaborolane according to the method described for Intermediate 37. LCMS (ES+) [M+H]⁺ 715, RT 1.28 minutes (method 5).

### Intermediate 40

### Ethyl 4-[7-(N-[(tert-butoxy)carbonyl]-N-{[3-(methanesulfonyl)phenyl]methyl}amino)-3-(3,4-dimethoxyphenyl)-2-methylpyrazolo[1,5-a]pyrimidin-5-yl]piperazine-1-carboxylate

A mixture of Intermediate 15 (1 eq), tripotassium phosphate (2.93 eq) and (3,4-dimethoxyphenyl)boronic acid (1.5 eq) in 1,4-dioxane (4 mL) and water (0.56 mL) was placed in a 20 mL pressure tube. The reaction mixture was purged with nitrogen gas for 10 minutes. Tetrakis(triphenylphosphine)palladium(0) (0.05 eq) was added, then the reaction mixture was purged with nitrogen gas, sealed and heated at 100° C. with stirring for 5 h. Upon cooling to r.t., the reaction mixture was diluted with EtOAc (40 mL) and dried with anhydrous magnesium sulfate, then filtered and concentrated in vacuo. The residue was dried at 40° C. for 18 h to afford the title compound. LCMS (ES+) [M+H]⁺ 709, RT 1.31 minutes (method 5).

### Intermediate 41

### Ethyl 4-[7-(N-[(tert-butoxy)carbonyl]-N-{[3-(methanesulfonyl)phenyl]methyl}amino)-3-[3-(methanesulfonyl)-4-methoxyphenyl]-2-methylpyrazolo[1,5-a]pyrimidin-5-yl]-piperazine-1-carboxylate

Prepared from Intermediate 15 and 2-[3-(methanesulfonyl)-4-methoxyphenyl]-4,4,5,5-tetramethyl-1,3,2-dioxaborolane according to the method described for Intermediate 40. LCMS (ES+) [M+H]⁻ 757, RT 1.21 minutes (method 5).

### Intermediate 42

### Ethyl 4-[7-(N-[(tert-butoxy)carbonyl]-N-{[3-(methanesulfonyl)phenyl]methyl}amino)-3-[3-(difluoromethoxy)-4-methoxyphenyl]-2-methylpyrazolo[1,5-a]pyrimidin-5-yl]-piperazine-1-carboxylate

Prepared from Intermediate 15 and 2-[3-(difluoromethoxy)-4-methoxyphenyl]-4,4,5,5-tetramethyl-1,3,2-dioxaborolane according to the method described for Intermediate 40. LCMS (ES+) [M+H]⁻ 745, RT 1.34 minutes (method 5).

### Intermediate 43

### Ethyl 4-[7-(N-[(tert-butoxy)carbonyl]-N-{[3-(methanesulfonyl)phenyl]methyl}amino)-3-(1,3-dimethyl-1H-indazol-5-yl)-2-methylpyrazolo[1,5-a]pyrimidin-5-yl]piperazine-1-carboxylate

A mixture of Intermediate 15 (95%, 100 mg, 0.15 mmol), Intermediate 19 (61 mg, 0.22 mmol) and tripotassium phosphate (0.44 mL) in 1,4-dioxane (4 mL) and water (0.56 mL) in a 20 mL pressure tube was degassed with nitrogen gas for 10 minutes. Tetrakis-(triphenylphosphine)palladium(0) (8.4 mg, 0.007 mmol) was added. The reaction mixture was purged with nitrogen gas, then sealed and heated at 100° C. with stirring for 5 h. Upon cooling to r.t., the reaction mixture was diluted with EtOAc (40 mL) and dried with anhydrous magnesium sulfate, then filtered and concentrated in vacuo. The residue was purified by flash column chromatography on silica (gradient elution with 0-100% EtOAc/heptane followed by 0-100% MeOH/DCM) to afford the title compound (60 mg, 55%) as a yellow oil. LCMS (ES+) [M+H]⁺ 717, RT 1.43 minutes (method 5).

### Intermediate 44

### 7-Methyl-2-(methylsulfanyl)pyrazolo[1,5-a][1,3,5]triazin-4-amine

3-Methyl-1H-pyrazol-5-amine (20 g, 205.9 mmol) was dissolved in ethanol (500 mL). Piperidine (0.61 mL, 6.2 mmol) was added, followed by dimethyl N-cyanocarbono-dithioimidate (33.1 g, 227 mmol). The reaction mixture was heated at reflux (external temperature 90° C.) with stirring for 3 h, then allowed to cool. The precipitate that formed was collected by filtration and washed with ethanol. The filtrate was concentrated in vacuo. The resulting solid was recrystallized with ethanol, then collected by filtration and combined with the initial precipitate. The filtrate was subjected to a repetition of the foregoing procedure and combined to afford further material (30.4 g). The filtrate was concentrated to 100 mL solution and allowed to stand for 18 h. The solid that formed was collected by filtration and washed with ethanol to afford further material (5.28 g). The batches were combined to afford the title compound (36 g, 89%) as a white solid. δ_{H} (DMSO-d₆, 250 MHz) 8.61-7.98 (m, 2H), 6.05 (s, 1H), 2.44 (s, 3H), 2.34 (s, 3H).

### Intermediate 45

### 8-Iodo-7-methyl-2-(methylsulfanyl)pyrazolo[1,5-a][1,3,5]triazin-4-amine

Intermediate 44 (7.7 g, 39 mmol) and 1-iodopyrrolidine-2,5-dione (10.7 g, 47 mmol) were suspended in DCM (150 mL). The pale pink suspension was stirred for 3 h, then concentrated in vacuo. Water (200 mL) was added to the resulting pale pink solid and the mixture was sonicated. The water was decanted and ethanol (100 mL) was added. The mixture was sonicated to give a white suspension. The resulting solid was collected by filtration and washed with ethanol (2×50 mL) to afford the title compound (12 g, 95%) as a white solid. δ_{H} (CDCl₃, 500 MHz) 2.62 (s, 3H), 2.43 (s, 3H).

### Intermediate 46

### 8-Iodo-2-(methanesulfonyl)-7-methylpyrazolo[1,5-a][1,3,5]triazin-4-amine

Intermediate 45 (12 g, 37.4 mmol) was dissolved in DMF (125 mL) and cooled to 0° C. with stirring. MCPBA (70%, 19.3 g, 78.5 mmol) was added in portions over 10 minutes. DMF (50 mL) was added and the reaction mixture was stirred at r.t. for 18 h. MCPBA (70%, 2 g, 8.11 mmol) was added and the reaction mixture was stirred for a further 3 h. Saturated aqueous sodium hydrogen carbonate solution (200 mL) was added whilst stirring. The resulting solid was collected by filtration, and washed with water, to afford the title compound (12.2 g, 92%) as a white solid. δ_{H} (DMSO-d₆, 500 MHz) 9.45 (s, 1H), 9.06 (s, 1H), 3.36-3.27 (m, 3H), 2.42 (s, 3H). LCMS (ES+) [M+H]⁺ 353.9, RT 1.06 minutes (method 7).

### Intermediate 47

### 8-Iodo-7-methyl-2-(morpholin-4-yl)pyrazolo[1,5-a][1,3,5]triazin-4-amine

Intermediate 46 (5 g, 14.2 mmol) and morpholine (6.19 mL, 70.8 mmol) were heated in 1,4-dioxane (50 mL) at 100° C. for 30 minutes. The reaction mixture was cooled, then water (100 mL) was added. The solid precipitate was collected by filtration and washed with water (2×20 mL) to afford the title compound (5 g, 98%) as a white solid. δ_{H} (DMSO-d₆, 500 MHz) 8.34-7.53 (m, 2H), 3.74-3.67 (m, 4H), 3.67-3.60 (m, 4H), 2.25 (s, 3H).

### Intermediate 48

### 3-(3,4-Dimethoxyphenyl)-5-hydroxy-2-methyl-4H-pyrazolo[1,5-a]pyrimidin-7-one

4-(3,4-Dimethoxyphenyl)-3-methyl-1H-pyrazol-5-amine (2 g, 8.57 mmol) in ethanol (50 mL) was treated with diethyl malonate (1.53 g, 9.43 mmol) and sodium ethoxide (8.33 g, 25.72 mmol) and heated at reflux for 20 h. To the precipitate was added diethyl ether and the slurry was stirred for 5 minutes. The solid was collected by filtration and washed with diethyl ether, then taken up in water and washed with EtOAc. The organic layers were discarded and the aqueous layer was acidified dropwise with concentrated hydrochloric acid. The mixture was allowed to stand overnight, then the solid was collected by filtration and air dried, affording the title compound (1.5 g, 58%) as a white solid. LCMS (ES+) [M+H]⁺ 302.1, RT 0.65 minutes (method 3).

### Intermediate 49

### 5,7-Dichloro-3-(3,4-dimethoxyphenyl)-2-methylpyrazolo[1,5-a]pyrimidine

Intermediate 48 (1.5 g, 5.0 mmol) was slurried in phosphoryl chloride (5 mL) with N,N-diethylaniline (0.75 g, 5.0 mmol). The mixture was heated at 80° C. for 30 minutes, then at 100° C. for 3 h. The resulting dark red solution was concentrated in vacuo. The resulting red oil was taken up in EtOAc and washed with water, then dried over anhydrous sodium sulfate and concentrated in vacuo, to afford the title compound (0.92 g, 55%) as a yellow oil that solidified upon concentration from diethyl ether. δ_{H} (CDCl₃, 300 MHz) 7.29-7.15 (m, 2H), 7.10-6.95 (m, 1H), 6.87 (s, 1H), 3.92 (s, 6H), 2.62 (s, 3H).

### Intermediate 50

### 3-Methyl-1-(Methanesulfinyl)benzene

1-Methyl-3-(methylsulfanyl)benzene (5 g, 36.2 mmol) was stirred in MeOH (250 mL) and THF (210 mL). Sodium periodate (10 g, 47 mmol) in water (155 mL) was added and the reaction mixture was stirred for 22 h at r.t. To the resulting white suspension was added saturated aqueous sodium chloride solution (100 mL), followed by water (850 mL). The aqueous layer of the resulting clear solution was extracted with EtOAc (5×250 mL). The organic layers were dried over anhydrous sodium sulfate and concentrated in vacuo. The resulting yellow solid/liquid mix was triturated with DCM, and the soluble organic material was purified by flash column chromatography on silica (gradient elution with 0-100% EtOAc/heptane), to afford the title compound (4.8 g, 87%) as a clear pale yellow liquid. δ_{H} (DMSO-d₆, 500 MHz) 7.53-7.43 (m, 3H), 7.38-7.32 (m, 1H), 2.72 (s, 3H), 2.39 (s, 3H).

### Intermediate 51

### Imino(methyl)(3-methylphenyl)-λ⁶-sulfanone

To Intermediate 50 (4.8 g, 31 mmol) in DCM (100 mL) were added 2,2,2-trifluoroacetamide (7 g, 62 mmol), magnesium oxide (5 g, 124 mmol), (diacetoxyiodo)-benzene (15 g, 47 mmol) and dirhodium tetraacetate (344 mg, 0.79 mmol). The reaction mixture was stirred for 20 h at r.t., then filtered through Kieselguhr and washed through with DCM. The filtrate was concentrated in vacuo. The resulting brown oil was dissolved in MeOH (50 mL) and potassium carbonate (21.5 g, 155 mmol) was added. The reaction mixture was stirred at r.t. for 2 h, then the suspension was filtered through Kieselguhr and washed with MeOH. The filtrate was concentrated in vacuo and the resulting solid was dry-loaded onto excess silica using MeOH. The material was purified by dry flash chromatography (gradient elution, 0-100% EtOAc/heptane followed by 0-2% MeOH/EtOAc) to afford the title compound (4 g, 72% at 95% purity) as an orange oil. δ_{H} (DMSO-d₆, 250 MHz) 7.80-7.68 (m, 2H), 7.53-7.43 (m, 2H), 4.13 (s, 1H), 3.04 (d, J 1.0 Hz, 3H), 2.40 (s, 3H). LCMS (ES+) [M+H]^{|} 169.90, RT 0.78 minutes (method 5).

### Intermediate 52

### tert-Butyl N-[methyl(3-methylphenyl)oxo-λ⁶-sulfanylidene]carbamate

To sodium hydride (60%, 1.42 g, 35.5 mmol) was added dry THF (25 mL) under nitrogen and the reaction mixture was cooled to 0° C. Intermediate 51 (3 g, 17.73 mmol) in dry THF (25 mL) was added dropwise. The white suspension was stirred for 1 h with warming to r.t. Di-tert-butyl dicarbonate (7.74 g, 35.45 mmol) was added as a solid. The reaction mixture was stirred at r.t. for 2 h, then carefully quenched with saturated aqueous ammonium chloride solution (50 mL). The reaction mixture was diluted with water (50 mL) and extracted with DCM (100 mL, then 50 mL). The organic layers were combined and washed with saturated aqueous sodium chloride solution (30 mL), then dried over anhydrous sodium sulfate and concentrated in vacuo. The resulting yellow oil (8 g) was purified by flash column chromatography on silica (gradient elution with 0-100% tert-butyl methyl ether in heptane) to afford the title compound (4 g, 84%) as a white solid. δ_{H} (DMSO-d₆, 250 MHz) 7.80-7.67 (m, 2H), 7.61-7.51 (m, 2H), 3.37 (s, 3H), 2.42 (s, 3H), 1.24 (s, 9H).

### Intermediate 53

### tert-Butyl N-{[3-(bromomethyl)phenyl](methyl)oxo-λ⁶-sulfanylidene}carbamate

To Intermediate 52 (4 g, 14.85 mmol) in acetonitrile (150 mL) was added N-bromosuccinimide (2.64 g, 14.85 mmol), followed by 2,2′-azobis(2-methylpropionitrile) (0.24 g, 1.49 mmol). The reaction mixture was heated at 90° C. with stirring for 1.5 h, then concentrated in vacuo. EtOAc (150 mL) was added, and the residue was washed with water (2×50 mL). The organic layers were dried over anhydrous sodium sulfate and concentrated in vacuo. Purification by flash column chromatography on silica (gradient elution with 0-100% tert-butyl methyl ether in heptane) afforded the title compound (3 g, 52% at 90% purity) as a clear oil. δ_{H} (DMSO-d₆, 250 MHz) 8.03 (t, J 1.7 Hz, 1H), 7.92-7.78 (m, 2H), 7.67 (t, J 7.8 Hz, 1H), 4.83 (s, 2H), 3.39 (s, 3H), 1.23 (s, 9H).

### Intermediate 54

### tert-Butyl N-({3-[(1,3-dioxo-2,3-dihydro-1H-isoindol-2-yl)methyl]phenyl}(methyl)oxo-λ⁶-sulfanylidene)carbamate

To Intermediate 53 (90%, 3.9 g, 10.08 mmol) in DMF (20 mL) was added potassium phthalimide (3 g, 16.2 mmol) and the suspension was stirred for 2 h. The reaction mixture was diluted with water (150 mL) and sonicated. The resulting sticky white gum was extracted with EtOAc (150 mL). The organic phase was washed with water (3×50 mL) and 5% aqueous lithium chloride solution (50 mL), then dried over anhydrous sodium sulfate and concentrated in vacuo. The resulting white solid was triturated with 70% EtOAc/heptane and collected by filtration to afford the title compound (2.9 g, 69%) as a white solid. δ_{H} (DMSO-d₆, 500 MHz) 7.91 (dd, J 5.3, 3.2 Hz, 2H), 7.89-7.82 (m, 4H), 7.71 (d, J 7.7 Hz, 1H), 7.65 (t, J 7.7 Hz, 1H), 4.89 (s, 2H), 3.35 (s, 3H), 1.15 (s, 9H).

### Intermediate 55

### tert-Butyl N-{[3-(aminomethyl)phenyl](methyl)oxo-λ⁶-sulfanylidene}carbamate

Intermediate 54 (800 mg, 1.93 mmol) was heated in ethanol (10 mL) and hydrazine hydrate (0.47 mL, 9.65 mmol) was added. The reaction mixture was heated at 80° C. with stirring in a 20 mL sealed tube for 1 h. The resulting white solid was diluted with MeOH and filtered. The solid was washed with MeOH and the filtrate was concentrated in vacuo. To the resulting white solid was added DCM (50 mL) and the mixture was filtered to remove solid phthalazine-1,4-dione, washing with further DCM (50 mL). The filtrate was concentrated in vacuo. The resulting oily solid was purified by SCX column (5 g, load and wash with MeOH, elution with 3.5M NH₃/MeOH) to afford the title compound (500 mg, 91%) as a clear colourless oil. δ_{H} (DMSO-d₆, 500 MHz) 7.92 (s, 1H), 7.75 (d, J 7.8 Hz, 1H), 7.69 (d, J 7.6 Hz, 1H), 7.59 (t, J 7.7 Hz, 1H), 3.83 (s, 2H), 3.37 (s, 3H), 1.98 (br s, 2H), 1.26 (s, 9H).

### Intermediate 56

### 2-({3-[Imino(methyl)oxo-λ⁶-sulfanyl]phenyl}methyl)-2,3-dihydro-1H-isoindole-1,3-dione

Intermediate 54 (1.4 g, 3.38 mmol) was dissolved in DCM (20 mL) and TFA (2.8 ml, 36.59 mmol) was added. The reaction mixture was stirred for 1.5 h, then quenched with saturated aqueous sodium hydrogen carbonate solution (50 mL), separated and extracted with DCM (50 mL). The organic phases were combined and washed with saturated sodium chloride solution, then dried over anhydrous sodium sulfate and concentrated in vacuo, to afford the title compound (1.12 g, 98%) as a white solid. δ_{H} (DMSO-d₆, 500 MHz) 7.94-7.89 (m, 2H), 7.89-7.85 (m, 3H), 7.85-7.82 (m, 1H), 7.62-7.53 (m, 2H), 4.87 (s, 2H), 4.20 (s, 1H), 3.04 (d, J 0.8 Hz, 3H).

### Intermediate 57

### 2-({3-[Methyl(methylimino)oxo-λ⁶-sulfanyl]phenyl}methyl)-2,3-dihydro-1H-isoindole-1,3-dione

Two 20 mL pressure tubes were charged with Intermediate 56 (0.5 g, 1.59 mmol). Formic acid (5 mL, 116.6 mmol) and formaldehyde (37% aqueous solution, 2.5 mL, 33.58 mmol) were added to each tube. The reaction mixtures were sealed and heated at 100° C. for 4.5 h. Further formaldehyde (37% aqueous solution, 1 mL, 13.43 mmol) was added to both mixtures, and both were heated at 100° C. for a further 18 h. The reaction mixtures were allowed to cool and were combined. The combined reaction mixture was adjusted to pH 8 using saturated aqueous sodium hydrogen carbonate solution (200 mL). The resulting white precipitate was extracted with DCM (100 mL). The aqueous layer was extracted with further DCM (2×50 mL), then the organic phases were combined, dried over anhydrous sodium sulfate and concentrated in vacuo. The resulting white solid (900 mg) was purified by flash column chromatography on silica (gradient elution with 0-100% EtOAc/heptane) to afford the title compound (778 mg, 74%) as a white solid. δ_{H} (DMSO-d₆, 250 MHz) 7.97-7.83 (m, 4H), 7.81-7.76 (m, 1H), 7.76-7.69 (m, 1H), 7.65-7.53 (m, 2H), 4.88 (s, 2H), 3.09 (s, 3H), 2.43 (s, 3H). LCMS (ES+) [M+H]⁺ 329, RT 1.61 minutes (method 8).

### Intermediate 58

### {3-[Methyl(methylimino)oxo-λ⁶-sulfanyl]phenyl}methanamine

To a suspension of Intermediate 57 (778 mg, 2.37 mmol) in ethanol (20 mL) was added hydrazine hydrate (0.58 mL, 11.85 mmol). The reaction mixture was heated to 80° C. and stirred for 1 h. A white precipitate formed, and the reaction mixture was diluted with DCM (100 mL) and filtered. The precipitate was washed with DCM (100 mL) and the filtrate was concentrated in vacuo. The resulting white oily solid was suspended in DCM (50 mL) and filtered again, washing with further DCM (50 mL). The filtrate was concentrated in vacuo. The resulting yellow oil was purified by SCX column (10 g, loaded and washed with MeOH, elution with 3.5M NH₃ in MeOH) to afford the title compound (418 mg, 89%) as a yellow oil. δ_{H} (DMSO-d₆, 500 MHz) 7.82 (s, 1H), 7.65 (d, J 7.6 Hz, 1H), 7.62 (d, J 7.8 Hz, 1H), 7.55 (t, J 7.6 Hz, 1H), 3.82 (s, 2H), 3.09 (s, 3H), 2.46 (s, 3H).

### Intermediate 59

### 5-Chloro-3-(3,4-dimethoxyphenyl)-N-{[3-(N,S-dimethylsulfonimidoyl)phenyl]methyl}-2-methylpyrazolo[1,5-a]pyrimidin-7-amine

To a suspension of Intermediate 49 (250 mg, 0.74 mmol) and Intermediate 58 (147 mg, 0.74 mmol) in 1-butanol (5 mL) was added DIPEA (0.39 mL, 2.22 mmol). The reaction mixture was heated at 70° C. with stirring for 1 h, then cooled, diluted with DCM (30 mL) and washed with water (20 mL). The organic layer was dried over anhydrous sodium sulfate and the solvent was removed in vacuo. Purification by flash chromatography on silica (gradient elution with 50-100% EtOAc/isohexane, followed by 0-5% MeOH/EtOAc) afforded the title compound (205 mg, 47%). δ_{H} (DMSO-d₆, 300 MHz) 9.09 (t, J 6.6 Hz, 1H), 7.98-7.89 (m, 1H), 7.78-7.68 (m, 2H), 7.66-7.53 (m, 1H), 7.24 (d, J 2.0 Hz, 1H), 7.16 (dd, J 8.3, 2.0 Hz, 1H), 7.03 (d, J 8.4 Hz, 1H), 6.23 (s, 1H), 4.75 (d, J 6.6 Hz, 2H), 3.82-3.73 (m, 6H), 3.10 (s, 3H), 2.56 (s, 3H), 2.44 (s, 3H). LCMS (ES+) [M+H]⁺ 500/502, RT 1.95 minutes (method 3).

### Intermediate 60

### tert-Butyl N-{[3-({[5-chloro-3-(3,4-dimethoxyphenyl)-2-methylpyrazolo[1,5-a]-pyrimidin-7-yl]amino}methyl)phenyl](methyl)oxo-λ⁶-sulfanylidene}carbamate

Prepared from Intermediate 49, Intermediate 55 and DIPEA according to the method described for Intermediate 59. δ_{H} (DMSO-d₆, 300 MHz) 9.12 (t, J 6.7 Hz, 1H), 8.05 (d, J 1.9 Hz, 1H), 7.87-7.79 (m, 2H), 7.67 (t, J 7.8 Hz, 1H), 7.23 (d, J 2.0 Hz, 1H), 7.15 (dd, J 8.3, 2.0 Hz, 1H), 7.03 (d, J 8.4 Hz, 1H), 6.21 (s, 1H), 4.76 (d, J 6.6 Hz, 2H), 3.78 (s, 6H), 3.36 (s, 3H), 2.56 (s, 3H), 1.13 (s, 9H). LCMS (ES+) [M-BOC+H]⁻ 486/488, RT 2.29 minutes (method 3).

### Intermediate 61

### tert-Butyl N-{[3-({[5-(4-acetylpiperazin-1-yl)-3-(3,4-dimethoxyphenyl)-2-methyl-pyrazolo[1,5-a]pyrimidin-7-yl]amino}methyl)phenyl](methyl)oxo-λ⁶-sulfanylidene}-carbamate

Prepared from Intermediate 60, 1-acetylpiperazine and DIPEA according to the method described for Example 37. LCMS (ES+) [M-BOC+H]⁺ 578, RT 1.64 minutes (method 3).

### Intermediate 62

### 3-Bromo-5-chloro-2-methyl-N-[(2-methylpyridin-4-yl)methyl]pyrazolo[1,5-a]pyrimidin-7-amine

Prepared from Intermediate 1, (2-methylpyridin-4-yl)methanamine and DIPEA according to the method described for Intermediate 4. δ_{H} (DMSO-d₆, 300 MHz) 9.10 (t, J 6.6 Hz, 1H), 8.37 (dd, J 5.2, 0.8 Hz, 1H), 7.20 (d, J 1.6 Hz, 1H), 7.14 (dd, J 5.2, 1.6 Hz, 1H), 6.19 (s, 1H), 4.63 (d, J 6.5 Hz, 2H), 2.43 (s, 3H), 2.42 (s, 3H). LCMS (ES+) [M+H]^{|} 366/368, RT 1.78 minutes (method 3).

### Intermediate 63

### tert-Butyl N-(3-bromo-5-chloro-2-methylpyrazolo[1,5-a]pyrimidin-7-yl)-N-[(2-methyl-pyridin-4-yl)methyl]carbamate

Prepared from Intermediate 62 according to the method described for Intermediate 7. δ_{H} (DMSO-d₆, 400 MHz) 8.34 (dd, J 5.1, 0.7 Hz, 1H), 7.36 (s, 1H), 7.28-7.20 (m, 1H), 7.15 (dd, J 5.3, 1.6 Hz, 1H), 4.95 (s, 2H), 2.46 (s, 3H), 2.41 (s, 3H), 1.27 (s, 9H). LCMS (ES+) [M+H]⁻ 466/468, RT 2.50 minutes (method 3).

### Intermediate 64

### tert-Butyl N-[5-(4-acetylpiperazin-1-yl)-3-bromo-2-methylpyrazolo[1,5-a]pyrimidin-7-yl]-N-[(2-methylpyridin-4-yl)methyl]carbamate

Prepared from Intermediate 63 and 1-acetylpiperazine according to the method described for Intermediate 10. δ_{H} (DMSO-d₆, 300 MHz) 8.37-8.26 (m, 1H), 7.29 (s, 1H), 7.23-7.12 (m, 1H), 6.79 (s, 1H), 4.91 (s, 2H), 3.79-3.46 (m, 8H), 2.40 (s, 3H), 2.32 (s, 3H), 2.04 (s, 3H), 1.28 (s, 9H). LCMS (ES+) [M+H]⁺ 558.2/560.1, RT 2.145 minutes (method 3).

### Intermediate 65

### tert-Butyl N-[5-(4-acetylpiperazin-1-yl)-3-(3,4-dimethoxyphenyl)-2-methylpyrazolo[1,5-a]pyrimidin-7-yl]-N-[(2-methylpyridin-4-yl)methyl]carbamate

Prepared from Intermediate 64 according to the method described for Intermediate 22. δ_{H} (DMSO-d₆, 300 MHz) 8.34 (d, J 5.0 Hz, 1H), 7.51 (d, J 2.0 Hz, 1H), 7.37-7.33 (m, 1H), 7.24-7.21 (m, 1H), 7.18 (dd, J 8.4, 2.0 Hz, 1H), 6.99 (d, J 8.5 Hz, 1H), 6.79 (s, 1H), 4.95 (s, 2H), 3.80 (s, 3H), 3.77 (s, 3H), 3.74-3.47 (m, 8H), 2.53 (s, 3H), 2.41 (s, 3H), 2.04 (s, 3H), 1.31 (s, 9H). LCMS (ES+) [M+H]⁺ 616, RT 2.16 minutes (method 3).

### Intermediate 66

### tert-Butyl N-[5-(4-acetylpiperazin-1-yl)-3-(1,3-dimethylindazol-5-yl)-2-methylpyrazolo-[1,5-a]pyrimidin-7-yl]-N-[(2-methylpyridin-4-yl)methyl]carbamate

Prepared from Intermediate 64 and Intermediate 19 according to the method described for Intermediate 22. LCMS (ES+) [M+H]⁺ 624, RT 2.17 minutes (method 3).

### Intermediate 67

### tert-Butyl N-{5-(4-acetylpiperazin-1-yl)-3-[4-chloro-3-(methylcarbamoyl)phenyl]-2-methylpyrazolo[1,5-a]pyrimidin-7-yl}-N-[(2-methylpyridin-4-yl)methyl]carbamate

Prepared from Intermediate 64 and Intermediate 21 according to the method described for Intermediate 22. δ_{H} (DMSO-d₆, 300 MHz) 8.36 (dd, J 10.4, 4.8 Hz, 2H), 7.89 (d, J 2.3 Hz, 1H), 7.83 (dd, J 8.5, 2.3 Hz, 1H), 7.50 (d, J 8.4 Hz, 1H), 7.34 (s, 1H), 7.22 (d, J 5.2 Hz, 1H), 6.84 (s, 1H), 4.95 (s, 2H), 3.77-3.48 (m, 8H), 2.77 (d, J 4.6 Hz, 3H), 2.54 (s, 3H), 2.41 (s, 3H), 2.05 (s, 3H), 1.30 (s, 9H). LCMS (ES+) [M+H]⁺ 647/649, RT 1.97 minutes (method 3).

### Intermediate 68

### tert-Butyl N-[5-(4-acetylpiperazin-1-yl)-3-(1,3-dimethylindazol-6-yl)-2-methylpyrazolo-[1,5-a]pyrimidin-7-yl]-N-{[3-(methylsulfonyl)phenyl]methyl}carbamate

A suspension of Intermediate 14 (150 mg, 0.24 mmol) and 1,3-dimethyl-6-(4,4,5,5-tetramethyl-1,3,2-dioxaborolan-2-yl)indazole (74 mg, 0.26 mmol) in 1,4-dioxane (2 mL) and 1M aqueous sodium carbonate solution (0.72 mL) was degassed using a stream of nitrogen for 5 minutes. [1,1′-Bis(diphenylphosphino)ferrocene]dichloro-palladium(II) complex with DCM (20 mg, 0.024 mmol) was added and the mixture was degassed for a further 5 minutes. The reaction mixture was sealed and heated at 110° C. under microwave irradiation for 1.5 h. The reaction mixture was diluted with DCM (10 mL) and washed with water (5 mL). The organic phase was dried over anhydrous sodium sulfate and concentrated in vacuo. The residue was purified by flash column chromatography on silica (gradient elution with 0-10% MeOH/DCM) to afford the title compound (129 mg, 37%). LCMS (ES+) [M+H]⁺ 687, RT 2.34 minutes (method 3).

### Intermediate 69

### 5-Chloro-3-(3,4-dimethoxyphenyl)-2-methyl-N-[(3-methyl-1,2,4-oxadiazol-5-yl)methyl]-pyrazolo[1,5-a]pyrimidin-7-amine

Prepared from Intermediate 49, [(3-methyl-1,2,4-oxadiazol-5-yl)methyl]amine hydrochloride and DIPEA according to the method described for Intermediate 4. δ_{H} (DMSO-d₆, 300 MHz) 8.85 (t, J 6.6 Hz, 1H), 7.30-7.23 (m, 1H), 7.21-7.13 (m, 1H), 7.08-6.96 (m, 1H), 6.43 (s, 1H), 5.01 (d, J 6.6 Hz, 2H), 3.80 (s, 6H), 2.55 (s, 3H), 2.32 (s, 3H). LCMS (ES+) [M+H]⁺ 415.2/417.2, RT 2.05 minutes (method 3).

### Intermediate 70

### N-Benzyl-3-bromo-5-chloro-2-methylpyrazolo[1,5-a]pyrimidin-7-amine

To Intermediate 1 (400 mg, 1.42 mmol) in 2-propanol (3 mL) in a 20 mL pressure tube were added DIPEA (0.5 mL, 2.85 mmol) and benzylamine (0.17 mL, 1.57 mmol). The reaction mixture was sealed and heated at 80° C. with stirring for 45 minutes. The reaction mixture was diluted with EtOAc (60 mL) and washed with water (2×40 mL). The organic layers were dried over anhydrous sodium sulfate and concentrated in vacuo. The resulting oil was sonicated in heptane and concentrated in vacuo to afford the title compound (486 mg, 97%) as a white solid. δ_{H} (DMSO-d₆, 250 MHz) 7.44-7.15 (m, 6H), 6.20 (s, 1H), 4.63 (s, 2H), 2.40 (s, 3H).

### Intermediate 71

### tert-Butyl N-benzyl-N-(3-bromo-5-chloro-2-methylpyrazolo[1,5-a]pyrimidin-7-yl)-carbamate

Intermediate 70 (486 mg, 1.38 mmol) and di tert-butyl dicarbonate (542 mg, 2.49 mmol) were dissolved in DCM (20 mL). 4-(Dimethylamino)pyridine (17 mg, 0.14 mmol) was added and the reaction mixture was stirred for 1 h. Imidazole (200 mg, 2.94 mmol) was added to remove excess di tert-butyl dicarbonate, and the reaction mixture was stirred for 30 minutes. The reaction mixture was diluted with DCM (50 mL) and washed with 0.5M aqueous hydrochloric acid (2×40 mL) and saturated aqueous sodium chloride solution, then dried over anhydrous sodium sulfate and concentrated in vacuo. The resulting clear oil was purified by flash column chromatography on silica (gradient elution with 0-40% EtOAc/heptane) to afford the title compound (682 mg, 98% at 90% purity). δ_{H} (DMSO-d₆, 500 MHz) 7.34-7.17 (m, 6H), 4.99 (s, 2H), 2.44 (s, 3H), 1.28 (s, 9H).

### Intermediate 72

### Ethyl 4-{7-[N-benzyl-N-(tert-butoxycarbonyl)amino]-3-bromo-2-methylpyrazolo[1,5-a]-pyrimidin-5-yl}piperazine-1-carboxylate

To Intermediate 71 (90%, 0.68 g, 1.36 mmol) in acetonitrile (10 mL) in a 20 mL pressure tube were added DIPEA (0.83 mL, 4.76 mmol) and ethyl piperazine-1-carboxylate (0.4 mL, 2.72 mmol). The reaction mixture was sealed and heated at 90° C. with stirring for 3 h. The reaction mixture was diluted with EtOAc (50 mL), and washed with water (40 mL) and 0.5M aqueous hydrochloric acid (40 mL), then dried over anhydrous sodium sulfate and concentrated in vacuo. The resulting yellow oil was purified by flash column chromatography on silica (gradient elution with 0-50% EtOAc/heptane). The resulting white foam (633 mg) was sonicated in heptane. To the resulting oily solid was added EtOAc. The resulting white solid was collected by filtration and washed with heptane. The filtrate was concentrated in vacuo and the residue was dissolved in EtOAc (2 mL). Heptane (10 mL) was added, and the resulting solution was concentrated in vacuo. To the resulting white solid precipitate was added further heptane, and the mixture was sonicated. The white solid was collected by filtration and added to the previous batch to afford the title compound (448 mg, 57%) as a white solid. δ_{H} (DMSO-d₆, 250 MHz) 7.39-7.14 (m, 5H), 6.61 (s, 1H), 4.93 (s, 2H), 4.07 (q, J7.1 Hz, 2H), 3.71-3.57 (m, 4H), 3.56-3.38 (m, 4H), 2.30 (s, 3H), 1.29 (s, 9H), 1.20 (t, J 7.1 Hz, 3H).

### Intermediate 73

### Ethyl 4-[7-(benzylamino)-3-(1,3-dimethyl-1H-indazol-5-yl)-2-methylpyrazolo[1,5-a]-pyrimidin-5-yl]piperazine-1-carboxylate

To Intermediate 72 (120 mg, 0.21 mmol) and Intermediate 19 (85 mg, 0.31 mmol) in a 20 mL pressure tube were added 1,4-dioxane (6 mL) and 1M aqueous tripotassium phosphate solution (0.63 mL). The mixture was purged with nitrogen for 3 minutes, then tetrakis(triphenylphosphine)palladium(0) (20 mg, 0.02 mmol) was added. The reaction mixture was sealed and heated at 100° C. with stirring for 3 h. The resulting black mixture was diluted with EtOAc (10 mL) and the aqueous layer was removed, then the organic layers were dried over anhydrous sodium sulfate and concentrated in vacuo. The black residue was dissolved in DCM (4 mL) and TFA (1 mL) was added. The reaction mixture was stirred for 2 h, then quenched with saturated aqueous sodium hydrogen carbonate solution (20 mL) and extracted with DCM (2×20 mL). The organic layers were dried over anhydrous sodium sulfate and concentrated in vacuo. The black residue was purified by flash column chromatography on silica (gradient elution with 0-100% EtOAc/heptane) to afford the title compound (62 mg, 52% at 95% purity) as a yellow oil/film. δ_{H} (DMSO-d₆, 500 MHz) 8.06 (t, J 6.6 Hz, 1H), 8.00-7.92 (m, 1H), 7.79 (dd, J 8.8, 1.5 Hz, 1H), 7.55 (d, J 8.8 Hz, 1H), 7.44 (d, J 7.2 Hz, 2H), 7.34 (t, J 7.6 Hz, 2H), 7.25 (t, J 7.3 Hz, 1H), 5.60 (s, 1H), 4.59 (d, J 6.6 Hz, 2H), 4.11-4.00 (m, 2H), 3.95 (s, 3H), 3.59-3.52 (m, 4H), 3.46-3.38 (m, 4H), 2.52 (s, 3H), 2.48 (s, 3H), 1.22-1.12 (m, 3H).

### Intermediate 74

### 5-Chloro-3-(3,4-dimethoxyphenyl)-2-methyl-N-[(5-methyl-1,3,4-oxadiazol-2-yl)methyl]-pyrazolo[1,5-a]pyrimidin-7-amine

To a suspension of Intermediate 49 (200 mg, 0.59 mmol) and (5-methyl-1,3,4-oxadiazol-2-yl)methanamine hydrochloride (186 mg, 1.18 mmol) in 1-butanol (2.5 mL) was added DIPEA (2.36 mmol, 0.41 mL). The reaction mixture was heated at 70° C. with stirring for 2 h, then cooled and concentrated in vacuo. A solid precipitate formed, which was filtered and washed with diethyl ether, to afford the title compound (158 mg, 64%) as a beige solid. δ_{H} (DMSO-d₆, 300 MHz) 8.90 (s, 1H), 7.25 (d, J 2.0 Hz, 1H), 7.17 (dd, J 8.4, 2.0 Hz, 1H), 7.05 (d, J 8.4 Hz, 1H), 6.36 (s, 1H), 4.90 (s, 2H), 3.79 (s, 6H), 2.54 (s, 3H), 2.48 (s, 3H).

### Intermediate 75

### tert-Butyl N-[5-(4-acetylpiperazin-1-yl)-2-methyl-3-(3-methyl-[1,2,4]triazolo[4,3-a]-pyridin-6-yl)pyrazolo[1,5-a]pyrimidin-7-yl]-N-{[3-(methylsulfonyl)phenyl]methyl}-carbamate

A suspension of Intermediate 14 (400 mg, 0.64 mmol) and (3-methyl-[1,2,4]-triazolo[4,3-a]pyridin-6-yl)boronic acid (171 mg, 0.97 mmol) in 1,4-dioxane (10 mL) and 1M aqueous potassium phosphate tribasic solution (1.93 mmol, 1.93 mL) was purged using a stream of nitrogen for 10 minutes. Tetrakis(triphenylphosphine)palladium(0) (74 mg, 0.064 mmol) was added and the mixture was purged for a further 5 minutes. The reaction mixture was sealed and heated at 100° C. under microwave irradiation for 3 h, then diluted with DCM (20 mL) and washed with water (10 mL). The organic phase was dried over anhydrous sodium sulfate and concentrated in vacuo. The crude material was purified by flash column chromatography on C18 silica (gradient elution with 0-100% acetonitrile in 10 mM ammonium bicarbonate solution, both containing 0.1% ammonia) to afford the title compound (146 mg, 34%) as a yellow solid. δ_{H} (DMSO-d₆, 300 MHz) 8.53 (t, J 1.3 Hz, 1H), 8.06 (t, J 1.8 Hz, 1H), 7.86-7.69 (m, 4H), 7.58 (t, J 7.8 Hz, 1H), 6.82 (s, 1H), 5.09 (s, 2H), 3.80-3.47 (m, 8H), 3.11 (s, 3H), 2.71 (s, 3H), 2.58 (s, 3H), 2.05 (s, 3H), 1.33 (s, 9H).

### Intermediate 76

### 5-Chloro-2-methylpyrazolo[1,5-a]pyrimidin-7-amine

To 5,7-dichloro-2-methylpyrazolo[1,5-a]pyrimidine (422 mg, 2.09 mmol), dissolved in 1,4-dioxane (5 mL), was added ammonium hydroxide solution (25%, 1.6 mL, 10 mmol). The reaction mixture was stirred under nitrogen at r.t. overnight, then concentrated in vacuo. The resulting cream-coloured solid was partitioned between EtOAc (40 mL) and saturated aqueous sodium hydrogen carbonate solution (20 mL). The organic phase was separated and dried, then filtered under reduced pressure. The solvent was removed in vacuo to afford the title compound (328 mg, 86.0%) as a pale grey solid. δ_{H} (DMSO-d₆, 300 MHz) 7.97 (s, 2H), 6.16 (s, 1H), 5.95 (s, 1H), 2.38 (s, 3H).

### Intermediate 77

### tert-Butyl 4-(7-amino-2-methylpyrazolo[1,5-a]pyrimidin-5-yl)piperazine-1-carboxylate

To Intermediate 76 (200 mg, 1.09 mmol) and tert-butyl piperazine-1-carboxylate (1.02 g, 5.4 mmol) was added ethanol (2 mL). The reaction mixture was sealed in a microwave vial, then heated at 140° C. under microwave irradiation microwave for 7 h. The solvent was removed in vacuo. The resulting pink solid was partitioned between 2M hydrochloric acid (10 mL) and EtOAc (25 mL). The organic layer was separated, then the aqueous layer was adjusted to pH 7-8 and extracted with further EtOAc (2×25 mL). The organic layers were combined, dried with anhydrous sodium sulfate, and filtered under reduced pressure, then the solvent was removed in vacuo. The resulting crude pale pink solid was purified by flash column chromatography on silica (gradient elution with 0-100% EtOAc/isohexane) to afford the title compound (86.5 mg, 24%) as a white solid. δ_{H} (DMSO-d₆, 300 MHz) 7.03 (s, 2H), 5.70 (s, 1H), 5.49 (s, 1H), 3.49-3.36 (m, 8H), 2.27 (s, 3H), 1.42 (s, 9H).

### Intermediate 78

### tert-Butyl N-[3-bromo-2-methyl-5-(6-oxo-1,3,4,7,8,8a-hexahydropyrrolo[1,2-a]pyrazin-2-yl)pyrazolo[1,5-a]pyrimidin-7-yl]-N-[(2,4-dimethylthiazol-5-yl)methyl]carbamate

To Intermediate 8 (500 mg, 1.03 mmol) in acetonitrile (5 mL) were added DIPEA (0.36 mL, 2.05 mmol) and 2,3,4,7,8,8a-hexahydro-1H-pyrrolo[1,2-a]pyrazin-6-one (144.0 mg, 1.03 mmol). The reaction mixture was heated at 80° C. for 6 h, then at 100° C. for 4 h. The reaction was cooled and partitioned between DCM and water, then the organic phase was separated and concentrated in vacuo. The residue was purified by flash column chromatography on silica (gradient elution with 50-100% EtOAc/isohexane followed by 15% MeOH/EtOAc) to afford the title compound (500 mg, 82%) as a gummy foam. LCMS (ES+) [M+H]⁺ 590/592, RT 2.22 minutes (method 3).

### Intermediate 79

### 1,3-Dimethyl-6-(4,4,5,5-tetramethyl-1,3,2-dioxaborolan-2-yl)imidazo[1,5-a]pyridine

To a dried flask were added 6-bromo-1,3-dimethylimidazo[1,5-a]pyridine (300 mg, 1.33 mmol), 2-isopropoxy-4,4,5,5-tetramethyl-1,3,2-dioxaborolane (0.30 ml, 1.4 mmol) and anhydrous THF (12 mL). The mixture was cooled to −78° C. before 1.6M n-butyllithium in hexanes (1.1 mL, 1.8 mmol) was added dropwise. After 40 minutes, additional 1.6M n-butyllithium in hexanes (50 μL, 0.1 mmol) was added. The mixture was warmed to ambient temperature before being quenched with 1M aqueous potassium phosphate solution (50 μL). After 15 minutes, the resultant precipitate was recovered by filtration, then washed with EtOAc (10 mL) and dried, to afford the title compound (491 mg, quantitative) as a yellow solid. δ_{H} (300 MHz, DMSO-d₆) 7.55-7.49 (m, 1H), 7.03 (dd, J 9.0, 1.2 Hz, 1H), 6.80 (d, J 9.0 Hz, 1H), 2.42 (s, 3H), 2.28 (s, 3H), 1.10-1.02 (m, 12H). LCMS (ES+) [M+H] 273, RT 1.62 minutes (method 3).

### Intermediate 80

### 3-Bromo-5-chloro-2-methyl-N-[(3-methyl-1,2,4-oxadiazol-5-yl)methyl]pyrazolo[1,5-a]-pyrimidin-7-amine

Intermediate 1 (2 g, 7.12 mmol) was dissolved in 2-propanol (30 mL), then (3-methyl-1,2,4-oxadiazol-5-yl)methanamine hydrochloride (1.06 g, 7.09 mmol) and DIPEA (3.70 mL, 21.36 mmol) were added. The reaction mixture was stirred at 80° C. for 4 h and left to stand at r.t. overnight, then concentrated in vacuo and partitioned between EtOAc (100 mL) and saturated aqueous sodium hydrogen carbonate solution (150 mL). The aqueous layer was separated and extracted with EtOAc (2×100 mL). The organic phases were combined and dried with anhydrous magnesium sulfate, then filtered under reduced pressure and concentrated in vacuo, to afford the title compound (2.45 g, 94%) as a pale pink solid. δ_{H} (500 MHz, CDCl₃) 6.95 (t, J 6.0 Hz, 1H), 6.02 (s, 1H), 4.80 (d, J 6.3 Hz, 2H), 2.46 (s, 3H), 2.43 (s, 3H). LCMS (ES+) [M+H]⁺ 357/359, RT 1.15 minutes (method 5).

### Intermediate 81

### tert-Butyl N-(3-bromo-5-chloro-2-methylpyrazolo[1,5-a]pyrimidin-7-yl)-N-[(3-methyl-1,2,4-oxadiazol-5-yl)methyl]carbamate

To a stirred solution of Intermediate 80 (2.45 g, 6.71 mmol) in 1,4-dioxane (150 mL) was added di-tert-butyl dicarbonate (2.93 g, 13.43 mmol), followed by 4-(dimethyl-amino)pyridine (82 mg, 0.67 mmol). The reaction mixture was stirred at r.t. for 18 h, then concentrated in vacuo and purified by flash column chromatography on silica (gradient elution with 0-100% EtOAc/heptane), to afford the title compound (2.86 g, 87%) as a yellow foam. δ_{H} (500 MHz, CDCl₃) 6.97 (s, 1H), 5.24 (s, 2H), 2.49 (s, 3H), 2.40 (s, 3H), 1.43 (s, 9H). LCMS (ES+) [M+H]⁺ 457/459, RT 1.36 minutes (method 5).

### Intermediate 82

### tert-Butyl N-[5-(4-acetylpiperazin-1-yl)-3-bromo-2-methylpyrazolo[1,5-a]pyrimidin-7-yl]-N-[(3-methyl-1,2,4-oxadiazol-5-yl)methyl]carbamate

A mixture of Intermediate 81 (1 g, 2.14 mmol), 1-acetylpiperazine (549 mg, 4.28 mmol) and DIPEA (1.31 mL, 7.49 mmol) in acetonitrile was heated at 90° C. for 18 h in a sealed tube. Upon cooling to r.t., the reaction mixture was concentrated in vacuo and purified by flash column chromatography on silica (gradient elution with 0-10% MeOH/DCM) to afford the title compound (1.23 g, 68%, at 65-70% purity) as a yellow-orange foam. LCMS (ES+) [M+H]⁻ 549/551, RT 1.24 minutes (method 5).

### Intermediate 83

### 5-Chloro-3-(3,4-dimethoxyphenyl)-N-[(4-methoxyphenyl)methyl]-2-methylpyrazolo[1,5-a]pyrimidin-7-amine

To a suspension of Intermediate 49 (2.0 g, 5.9 mmol) and 4-methoxybenzylamine (890 mg, 0.85 mL, 6.5 mmol) in 1-butanol (20 mL) was added DIPEA (2.3 g, 3.1 mL, 18 mmol). The reaction mixture was stirred at 70° C. for 2 h, then cooled to r.t. The solid was filtered and washed with DCM, then dried under reduced pressure, to afford the title compound (2.42 g, 93%) as a pale yellow powder. δ_{H} (300 MHz, DMSO-d₆) 8.89 (t, J 6.5 Hz, 1H), 7.35 (d, J 8.7 Hz, 2H), 7.24 (d, J 2.0 Hz, 1H), 7.20-7.12 (m, 1H), 7.03 (d, J 8.4 Hz, 1H), 6.96-6.84 (m, 2H), 6.13 (s, 1H), 4.56 (d, J 6.5 Hz, 2H), 3.78 (s, 6H), 3.72 (s, 3H), 2.54 (s, 3H).

### Intermediate 84

### tert-Butyl N-[5-chloro-3-(3,4-dimethoxyphenyl)-2-methylpyrazolo[1,5-a]pyrimidin-7-yl]-N-[(4-methoxyphenyl)methyl]carbamate

To a solution of Intermediate 83 (2.42 g, 5.51 mmol) in 1,4-dioxane (50 mL) was added 4-(dimethylamino)pyridine (54.4 mg, 0.441 mmol), followed by di-tert-butyl dicarbonate (1.64 g, 7.44 mmol). The reaction mixture was stirred at r.t. overnight, then diluted with DCM (100 mL) and washed with water (50 mL). The organic phase was separated and the solvent was removed in vacuo. The resulting crude yellow oil was purified by flash column chromatography on silica (gradient elution with 0-100% EtOAc/isohexane) to afford the title compound (2.09 g, 70.3%) as a yellow solid. δ_{H} (300 MHz, DMSO-d₆) 7.28-7.14 (m, 4H), 7.09-7.02 (m, 2H), 6.89-6.80 (m, 2H), 4.96 (s, 2H), 3.80 (s, 6H), 3.70 (s, 3H), 2.58 (s, 3H), 1.33 (s, 9H).

### Intermediate 85

### 5-Chloro-3-(3,4-dimethoxyphenyl)-2-methyl-N-[(2-methylpyridin-4-yl)methyl]pyrazolo-[1,5-a]pyrimidin-7-amine

To Intermediate 49 (7.1 g, 21 mmol) and (2-methylpyridin-4-yl)methanamine (3 g, 23.3 mmol) in acetonitrile (150 mL) was added DIPEA (8.1 g, 11 mL, 63 mmol). The reaction mixture was heated at 80° C. for 6 h, then cooled to r.t. and concentrated in vacuo. The solid was triturated with water and air-dried to afford the title compound (9.5 g, 96%). δ_{H} (300 MHz, DMSO-d₆) 9.07-8.83 (m, 1H), 8.39 (dd, J 5.1, 0.8 Hz, 1H), 7.26-7.20 (m, 2H), 7.20-7.11 (m, 2H), 7.04 (d, J 8.4 Hz, 1H), 6.11 (s, 1H), 4.65 (d, J 4.9 Hz, 2H), 3.79 (s, 6H), 2.56 (s, 3H), 2.44 (s, 3H).

### Intermediate 86

### tert-Butyl N-[5-chloro-3-(3,4-dimethoxyphenyl)-2-methylpyrazolo[1,5-a]pyrimidin-7-yl]-N-[(2-methylpyridin-4-yl)methyl]carbamate

To a solution of Intermediate 85 (2.29 g, 5.40 mmol) in 1,4-dioxane (50 mL) were added 4-(dimethylamino)pyridine (53.3 mg, 0.432 mmol) and di-tert-butyl dicarbonate (1.61 g, 7.29 mmol). The reaction mixture was stirred at r.t. overnight, then diluted with DCM (100 mL) and washed with water (50 mL). The organic layer was separated and concentrated in vacuo. The resulting yellow oil was purified by flash column chromatography on silica (gradient elution with 40-100% EtOAc/isohexane) to afford the title compound (2.19 g, 77%) as a yellow solid. δ_{H} (300 MHz, DMSO-d₆) 8.36 (dd, J 5.1, 0.8 Hz, 1H), 7.31-7.28 (m, 1H), 7.28 (s, 1H), 7.24 (d, J 2.0 Hz, 1H), 7.21-7.16 (m, 2H), 7.10-7.04 (m, 1H), 4.99 (s, 2H), 3.80 (s, 6H), 2.60 (s, 3H), 2.42 (s, 3H), 1.31 (s, 9H).

### Example 1

### 5-(3,3-Difluoroazetidin-1-yl)-3-(3,4-dimethoxyphenyl)-N-[(1,3-dimethyl-1H-pyrazol-5-yl)methyl]-2-methylpyrazolo[1,5-a]pyrimidin-7-amine

Intermediate 22 (1 eq) was dissolved in DCM/TFA (4:1). The reaction mixture was stirred at r.t. under a nitrogen atmosphere for 2-18 h, then concentrated in vacuo. To the residue was added saturated aqueous sodium hydrogen carbonate solution (20 mL) until the effervescence subsided. The aqueous layer was extracted into DCM (3×30 mL), then the combined organic layers were dried with anhydrous magnesium sulfate, filtered and concentrated in vacuo. The crude residue was purified by basic preparative HPLC, followed by flash column chromatography on silica (gradient elution with 0-100% MeOH/DCM), to afford the title compound. δ_{H} (DMSO-d₆, 500 MHz) 8.08 (t, J 6.2 Hz, 1H), 7.62 (d, J 2.0 Hz, 1H), 7.13 (dd, J 8.3, 2.0 Hz, 1H), 6.97 (d, J 8.4 Hz, 1H), 6.02 (s, 1H), 5.42 (s, 1H), 4.55 (d, J 6.2 Hz, 2H), 4.42 (t, J 12.4 Hz, 4H), 3.79 (s, 3H), 3.76 (s, 3H), 3.76 (s, 3H), 2.50 (s, 3H, obscured by DMSO peaks), 2.07 (s, 3H). LCMS (ES+) [M+H]⁺ 484, RT 3.29 minutes (method 1).

### Example 2

### 5-(3,3-Difluoroazetidin-1-yl)-N-[(2,5-dimethylpyrazol-3-yl)methyl]-3-[3-(methane-sulfonyl)-4-methoxyphenyl]-2-methylpyrazolo[1,5-a]pyrimidin-7-amine

Prepared from Intermediate 23 according to the method described for Example 1. The crude residue was purified by basic preparative HPLC to afford the title compound. δ_{H} (CD₃OD, 500 MHz) 8.60 (d, J 2.3 Hz, 1H), 8.02 (dd, J 8.7, 2.4 Hz, 1H), 7.29 (d, J 8.7 Hz, 1H), 6.12 (s, 1H), 5.38 (s, 1H), 4.63 (s, 2H), 4.42 (t, J 12.2 Hz, 4H), 4.03 (s, 3H), 3.82 (s, 3H), 3.26 (s, 3H), 2.54 (s, 3H), 2.19 (s, 3H). LCMS (ES+) [M+H] 532, RT 3.02 minutes (method 1).

### Example 3

### 5-(3 ,3-Difluoroazetidin-1-yl)-3-(1,3-dimethyl-1H-indazol-5-yl)-N-[(1,3-dimethyl-1H-pyrazol-5-yl)methyl]-2-methylpyrazolo[1,5-a]pyrimidin-7-amine

Prepared from Intermediate 24 according to the method described for Example 1. δ_{H} (DMSO-d₆, 500 MHz) 8.10 (t, J 6.2 Hz, 1H), 7.93 (s, 1H), 7.75 (dd, J 8.7, 1.5 Hz, 1H), 7.54 (d, J 8.3 Hz, 1H), 6.03 (s, 1H), 5.44 (s, 1H), 4.57 (d, J 6.2 Hz, 2H), 4.41 (t, J 12.4 Hz, 4H), 3.96 (s, 3H), 3.77 (s, 3H), 2.48 (s, 6H, obscured by DMSO peaks), 2.08 (s, 3H). LCMS (ES+) [M+H]⁺ 492, RT 3.29 minutes (method 1).

### Example 4

### 5-(3,3-Difluoroazetidin-1-yl)-3-[3-(difluoromethoxy)-4-methoxyphenyl]-N-[(1,3-dimethyl-1H-pyrazol-5-yl)methyl]-2-methylpyrazolo[1,5-a]pyrimidin-7-amine

Prepared from Intermediate 25 according to the method described for Example 2. δ_{H} (DMSO-d₆, 500 MHz) 8.12 (t, J 6.3 Hz, 1H), 7.76 (d, J 2.0 Hz, 1H), 7.52 (dd, J 8.6, 2.1 Hz, 1H), 7.27-6.88 (m, 2H), 6.02 (s, 1H), 5.44 (s, 1H), 4.55 (d, J 6.2 Hz, 2H), 4.41 (t, J 12.4 Hz, 4H), 3.84 (s, 3H), 3.76 (s, 3H), 2.50 (s, 3H, obscured by DMSO peaks), 2.07 (s, 3H). LCMS (ES+) [M+H]⁻ 520, RT 3.74 minutes (method 1).

### Example 5

### 3-(3,4-Dimethoxyphenyl)-N-[(1,3-dimethyl-1H-pyrazol-5-yl)methyl]-2-methyl-5-(2-oxa-6-azaspiro[3.3]heptan-6-yl)pyrazolo[1,5-a]pyrimidin-7-amine

Prepared from Intermediate 26 according to the method described for Example 2. δ_{H} (DMSO-d₆, 500 MHz) 7.89 (t, J 6.2 Hz, 1H), 7.71 (d, J 2.0 Hz, 1H), 7.14 (dd, J 8.3, 2.0 Hz, 1H), 6.96 (d, J 8.4 Hz, 1H), 5.99 (s, 1H), 5.21 (s, 1H), 4.71 (s, 4H), 4.53 (d, J 5.7 Hz, 2H), 4.14 (s, 4H), 3.81 (s, 3H), 3.76 (s, 3H), 3.75 (s, 3H), 2.48 (s, 3H), 2.07 (s, 3H). LCMS (ES+) [M+H]⁺ 490, RT 2.22 minutes (method 1).

### Example 6

### N-[(1,3-Dimethyl-1H-pyrazol-5-yl)methyl]-3-[3-(methanesulfonyl)-4-methoxyphenyl]-2-methyl-5-(2-oxa-6-azaspiro[3.3]heptan-6-yl)pyrazolo[1,5-a]pyrimidin-7-amine

Prepared from Intermediate 27 according to the method described for Example 2. δ_{H} (DMSO-d₆, 500 MHz) 8.49 (d, J 2.3 Hz, 1H), 8.03 (dd, J 8.7, 2.4 Hz, 1H), 7.96 (t, J 5.6 Hz, 1H), 7.31 (d, J 8.8 Hz, 1H), 5.99 (s, 1H), 5.24 (s, 1H), 4.72 (s, 4H), 4.61-4.48 (m, 2H), 4.16 (s, 4H), 3.97 (s, 3H), 3.75 (s, 3H), 3.26 (s, 3H), 2.50 (s, 3H, obscured by DMSO peaks), 2.07 (s, 3H). LCMS (ES+) [M+H]⁺ 538, RT 2.15 minutes (method 1).

### Example 7

### 3-(1,3-Dimethyl-1H-indazol-5-yl)-N-[(1,3-dimethyl-1H-pyrazol-5-yl)methyl]-2-methyl-5-(2-oxa-6-azaspiro[3.3]heptan-6-yl)pyrazolo[1,5-a]pyrimidin-7-amine

Prepared from Intermediate 28 according to the method described for Example 2. δ_{H} (DMSO-d₆, 500 MHz) 7.98-7.95 (m, 1H), 7.91 (t, J 6.0 Hz, 1H), 7.77 (dd, J 8.7, 1.5 Hz, 1H), 7.54 (d, J 8.8 Hz, 1H), 6.00 (s, 1H), 5.22 (s, 1H), 4.71 (s, 4H), 4.54 (d, J 5.8 Hz, 2H), 4.14 (s, 4H), 3.96 (s, 3H), 3.76 (s, 3H), 2.50 (s, 6H, obscured by DMSO peaks), 2.08 (s, 3H). LCMS (ES+) [M+H]⁻ 498, RT 2.19 minutes (method 1).

### Example 8

### 3-[3-(Difluoromethoxy)-4-methoxyphenyl]-N-[(1,3-dimethyl-1H-pyrazol-5-yl)methyl]-2-methyl-5-(2-oxa-6-azaspiro[3.3]heptan-6-yl)pyrazolo[1,5-a]pyrimidin-7-amine

Prepared from Intermediate 29 according to the method described for Example 2. δ_{H} (DMSO-d₆, 500 MHz) 7.93 (t, J 6.1 Hz, 1H), 7.79 (d, J 2.0 Hz, 1H), 7.53 (dd, J 8.6, 2.1 Hz, 1H), 7.25-6.88 (m, 2H), 5.99 (s, 1H), 5.22 (s, 1H), 4.71 (s, 4H), 4.53 (d, J 6.0 Hz, 2H), 4.14 (s, 4H), 3.84 (s, 3H), 3.75 (s, 3H), 2.47 (s, 3H), 2.07 (s, 3H). LCMS (ES+) [M+H]^{|} 526, RT 2.78 minutes (method 1).

### Example 9

### 1-(4-[3-(3,4-Dimethoxyphenyl)-7-{[(1,3-dimethyl-1H-pyrazol-5-yl)methyl]amino}-2-methylpyrazolo[1,5-a]pyrimidin-5-yl]piperazin-1-yl)ethan-1-one

Prepared from Intermediate 30 according to the method described for Example 2. δ_{H} (DMSO-d₆, 500 MHz) 7.87 (t, J 6.2 Hz, 1H), 7.57 (d, J 2.0 Hz, 1H), 7.16 (dd, J 8.3, 2.0 Hz, 1H), 6.98 (d, J 8.4 Hz, 1H), 6.01 (s, 1H), 5.69 (s, 1H), 4.59 (d, J 6.2 Hz, 2H), 3.79 (s, 3H), 3.76 (s, 3H), 3.75 (s, 3H), 3.69-3.63 (m, 2H), 3.61-3.56 (m, 2H), 3.55-3.50 (m, 4H), 2.49 (s, 3H), 2.07 (s, 3H), 2.04 (s, 3H). LCMS (ES+) [M+H]⁻ 519, RT 2.68 minutes (method 1).

### Example 10

### 1-[4-(7-{[(1,3-Dimethyl-1H-pyrazol-5-yl)methyl]amino}-3-[3-(methanesulfonyl)-4-methoxyphenyl]-2-methylpyrazolo[1,5-a]pyrimidin-5-yl)piperazin-1-yl]ethan-1-one

Prepared from Intermediate 31 according to the method described for Example 2. δ_{H} (DMSO-d₆, 500 MHz) 8.64 (d, J 2.4 Hz, 1H), 8.01 (dd, J 8.7, 2.4 Hz, 1H), 7.94 (t, J 6.3 Hz, 1H), 7.32 (d, J 8.8 Hz, 1H), 6.03 (s, 1H), 5.72 (s, 1H), 4.59 (d, J 6.2 Hz, 2H), 3.96 (s, 3H), 3.76 (s, 3H), 3.74-3.67 (m, 2H), 3.65-3.59 (m, 2H), 3.57-3.48 (m, 4H), 3.26 (s, 3H), 2.52 (s, 3H), 2.07 (s, 3H), 2.05 (s, 3H). LCMS (ES+) [M+H]⁻ 567, RT 2.51 minutes (method 1).

### Example 11

### 1-(4-[3-(1,3-Dimethyl-1H-indazol-5-yl)-7-{[(1,3-dimethyl-1H-pyrazol-5-yl)methyl]-amino}-2-methylpyrazolo[1,5-a]pyrimidin-5-yl]piperazin-1-yl)ethan-1-one

Prepared from Intermediate 32 according to the method described for Example 2. δ_{H} (DMSO-d₆, 500 MHz) 7.95 (s, 1H), 7.88 (t, J 6.3 Hz, 1H), 7.79 (dd, J 8.7, 1.5 Hz, 1H), 7.55 (d, J 8.8 Hz, 1H), 6.03 (s, 1H), 5.71 (s, 1H), 4.60 (d, J 6.2 Hz, 2H), 3.96 (s, 3H), 3.77 (s, 3H), 3.69-3.63 (m, 2H), 3.60-3.56 (m, 2H), 3.56-3.49 (m, 4H), 2.51 (s, 3H), 2.48 (s, 3H), 2.07 (s, 3H), 2.04 (s, 3H). LCMS (ES+) [M+H]^{|} 527, RT 2.68 minutes (method 1).

### Example 12

### 1-(4-{3-[3-(Difluoromethoxy)-4-methoxyphenyl]-7-{[(1,3-dimethyl-1H-pyrazol-5-yl)-methyl]amino}-2-methylpyrazolo[1,5-a]pyrimidin-5-yl}piperazin-1-yl)ethan-1-one

Prepared from Intermediate 33 according to the method described for Example 2. δ_{H} (DMSO-d₆, 500 MHz) 7.91 (t, J 6.4 Hz, 1H), 7.76 (d, J 2.0 Hz, 1H), 7.53 (dd, J 8.6, 2.1 Hz, 1H), 7.26-6.89 (m, 2H), 6.02 (s, 1H), 5.70 (s, 1H), 4.59 (d, J 6.2 Hz, 2H), 3.84 (s, 3H), 3.75 (s, 3H), 3.70-3.64 (m, 2H), 3.61-3.56 (m, 2H), 3.55-3.49 (m, 4H), 2.49 (s, 3H), 2.07 (s, 3H), 2.04 (s, 3H). LCMS (ES+) [M+H]⁺ 555, RT 3.13 minutes (method 1).

### Example 13

### 1-(4-[3-(3,4-Dimethoxyphenyl)-7-{[(2,4-dimethyl-1,3-thiazol-5-yl)methyl]amino}-2-methylpyrazolo[1,5-a]pyrimidin-5-yl]piperazin-1-yl)ethan-1-one

Prepared from Intermediate 34 according to the method described for Example 1. The crude residue was purified by neutral reverse phase chromatography (elution with acetonitrile/water), followed by basic preparative HPLC. The relevant fractions were combined and concentrated in vacuo, then the residue was triturated with hot heptane/DCM (4:1). The resulting precipitate was filtered and dried under vacuum at 40° C. for 18 h to afford the title compound. δ_{H} (DMSO-d₆, 500 MHz) 7.96 (t, J 6.4 Hz, 1H), 7.57 (d, J 1.9 Hz, 1H), 7.16 (dd, J 8.4, 2.0 Hz, 1H), 6.98 (d, J 8.4 Hz, 1H), 5.65 (s, 1H), 4.69 (d, J 6.4 Hz, 2H), 3.80 (s, 3H), 3.77 (s, 3H), 3.70-3.65 (m, 2H), 3.63-3.58 (m, 2H), 3.58-3.52 (m, 4H), 2.50 (s, 3H, obscured by DMSO peaks), 2.50 (s, 3H), 2.40 (s, 3H), 2.05 (s, 3H). LCMS (ES+) [M+H]⁻ 536, RT 2.80 minutes (method 1).

### Example 14

### 1-[4-(7-{[(2,4-Dimethyl-1,3-thiazol-5-yl)methyl]amino}-3-[3-(methanesulfonyl)-4-methoxyphenyl]-2-methylpyrazolo[1,5-a]pyrimidin-5-yl)piperazin-1-yl]ethan-1-one

Prepared from Intermediate 35 according to the method described for Example 1. The crude residue was purified by flash column chromatography on silica (gradient elution with 0-100% EtOAc/heptane, followed by 0-100% MeOH/DCM). The relevant fractions were combined and concentrated in vacuo, then purified by neutral reverse phase chromatography (elution with acetonitrile/water), to afford the title compound. δ_{H} (DMSO-d₆, 500 MHz) 8.64 (d, J 2.4 Hz, 1H), 8.07-7.99 (m, 2H), 7.33 (d, J 8.8 Hz, 1H), 5.68 (s, 1H), 4.69 (d, J 6.4 Hz, 2H), 3.97 (s, 3H), 3.74-3.69 (m, 2H), 3.67-3.62 (m, 2H), 3.59-3.51 (m, 4H), 3.27 (s, 3H), 2.50 (s, 6H, obscured by DMSO peaks), 2.40 (s, 3H), 2.06 (s, 3H). LCMS (ES+) [M+H]⁺ 584, RT 2.59 minutes (method 1).

### Example 15

### 1-[4-(3-[3-(Difluoromethoxy)-4-methoxyphenyl]-7-{[(2,4-dimethyl-1,3-thiazol-5-yl)-methyl]amino}-2-methylpyrazolo[1,5-a]pyrimidin-5-yl)piperazin-1-yl]ethan-1-one

Prepared from Intermediate 36 according to the method described for Example 14. δ_{H} (DMSO-d₆, 500 MHz) 7.99 (t, J 6.5 Hz, 1H), 7.77 (d, J 1.8 Hz, 1H), 7.53 (dd, J 8.6, 2.1 Hz, 1H), 7.26-6.88 (m, 2H), 5.66 (s, 1H), 4.69 (d, J 6.3 Hz, 2H), 3.85 (s, 3H), 3.70-3.64 (m, 2H), 3.60 (dd, J 5.8, 3.4 Hz, 2H), 3.54 (dd, J 6.5, 3.7 Hz, 4H), 2.51 (s, 3H, obscured by DMSO peaks), 2.48 (s, 3H), 2.39 (s, 3H), 2.06 (s, 3H). LCMS (ES+) [M+H]⁺ 572, RT 3.25 minutes (method 1).

### Example 16

### 1-{4-[3-(3,4-Dimethoxyphenyl)-7-({[3-(methanesulfonyl)phenyl]methyl}amino)-2-methylpyrazolo[1,5-a]pyrimidin-5-yl]piperazin-1-yl}ethan-1-one

Prepared from Intermediate 37 according to the method described for Example 1. The crude residue was purified by basic preparative HPLC. The relevant fractions were combined and concentrated in vacuo, then the residue was triturated with hot heptane/DCM (4:1). The resulting precipitate was filtered and dried under vacuum at 40° C. for 18 h to afford the title compound. δ_{H} (DMSO-d₆, 500 MHz) 8.22 (t, J 6.7 Hz, 1H), 8.08 (s, 1H), 7.84 (d, J 7.8 Hz, 1H), 7.80 (d, J 7.8 Hz, 1H), 7.63 (t, J 7.7 Hz, 1H), 7.57 (d, J 1.9 Hz, 1H), 7.16 (dd, J 8.4, 2.0 Hz, 1H), 6.98 (d, J 8.4 Hz, 1H), 5.65 (s, 1H), 4.71 (d, J 6.7 Hz, 2H), 3.79 (s, 3H), 3.77 (s, 3H), 3.69-3.60 (m, 2H), 3.58-3.53 (m, 2H), 3.52-3.46 (m, 4H), 3.21 (s, 3H), 2.50 (s, 3H, obscured by DMSO peaks), 2.03 (s, 3H). LCMS (ES+) [M+H] 579, RT 2.82 minutes (method 1).

### Example 17

### 1-{4-[3-[3-(Methanesulfonyl)-4-methoxyphenyl]-7-({[3-(methanesulfonyl)phenyl]-methyl}amino)-2-methylpyrazolo[1,5-a]pyrimidin-5-yl]piperazin-1-yl}ethan-1-one

Prepared from Intermediate 38 according to the method described for Example 2. δ_{H} (DMSO-d₆, 500 MHz) 8.63 (d, J 2.4 Hz, 1H), 8.28 (t, J 6.6 Hz, 1H), 8.09 (s, 1H), 8.01 (dd, J 8.7, 2.4 Hz, 1H), 7.84 (d, J 7.5 Hz, 1H), 7.80 (d, J 7.8 Hz, 1H), 7.64 (t, J 7.7 Hz, 1H), 7.33 (d, J 8.8 Hz, 1H), 5.67 (s, 1H), 4.71 (d, J 6.5 Hz, 2H), 3.97 (s, 3H), 3.71-3.65 (m, 2H), 3.63-3.56 (m, 2H), 3.53-3.46 (m, 4H), 3.26 (s, 3H), 3.21 (s, 3H), 2.54 (s, 3H), 2.04 (s, 3H). LCMS (ES+) [M+H]⁺ 627, RT 2.66 minutes (method 1).

### Example 18

### 1-{4-[3-[3-(Difluoromethoxy)-4-methoxyphenyl]-7-({[3-(methanesulfonyl)phenyl]-methyl}amino)-2-methylpyrazolo[1,5-a]pyrimidin-5-yl]piperazin-1-yl}ethan-1-one

Prepared from Intermediate 39 according to the method described for Example 2. δ_{H} (DMSO-d₆, 500 MHz) 8.25 (t, J 6.7 Hz, 1H), 8.08 (s, 1H), 7.84 (d, J 7.8 Hz, 1H), 7.79 (d, J 7.7 Hz, 1H), 7.76 (d, J 1.9 Hz, 1H), 7.63 (t, J 7.7 Hz, 1H), 7.53 (dd, J 8.6, 2.1 Hz, 1H), 7.25-6.87 (m, 2H), 5.66 (s, 1H), 4.71 (d, J 6.7 Hz, 2H), 3.84 (s, 3H), 3.69-3.61 (m, 2H), 3.58-3.53 (m, 2H), 3.49 (s, 4H), 3.21 (s, 3H), 2.51 (s, 3H, obscured by DMSO peaks), 2.03 (s, 3H). LCMS (ES+) [M+H]⁻ 615, RT 3.26 minutes (method 1).

### Example 19

### Ethyl 4-[3-(3,4-dimethoxyphenyl)-7-({[3-(methanesulfonyl)phenyl]methyl}amino)-2-methylpyrazolo[1,5-a]pyrimidin-5-yl]piperazine-1-carboxylate

Prepared from Intermediate 40 according to the method described for Example 2. δ_{H} (DMSO-d₆, 500 MHz) 8.22 (t, J 6.8 Hz, 1H), 8.08 (s, 1H), 7.83 (d, J 7.9 Hz, 1H), 7.79 (d, J 7.9 Hz, 1H), 7.63 (t, J 7.7 Hz, 1H), 7.56 (d, J 1.9 Hz, 1H), 7.15 (dd, J 8.4, 2.0 Hz, 1H), 6.98 (d, J 8.4 Hz, 1H), 5.65 (s, 1H), 4.70 (d, J 6.8 Hz, 2H), 4.07 (q, J7.1 Hz, 2H), 3.79 (s, 3H), 3.77 (s, 3H), 3.63-3.55 (m, 4H), 3.46-3.40 (m, 4H), 3.21 (s, 3H), 2.50 (s, 3H, obscured by DMSO peaks), 1.20 (t, J 7.1 Hz, 3H). LCMS (ES+) [M+H] 609, RT 3.41 minutes (method 1).

### Example 20

### Ethyl 4-[3-[3-(methanesulfonyl)-4-methoxyphenyl]-7-({[3-(methanesulfonyl)phenyl]-methyl}amino)-2-methylpyrazolo[1,5-a]pyrimidin-5-yl]piperazine-1-carboxylate

Prepared from Intermediate 41 according to the method described for Example 2. δ_{H} (DMSO-d₆, 500 MHz) 8.68 (d, J 2.3 Hz, 1H), 8.28 (t, J 6.7 Hz, 1H), 8.09 (s, 1H), 8.00 (dd, J 8.7, 2.4 Hz, 1H), 7.84 (d, J 8.0 Hz, 1H), 7.80 (d, J 7.6 Hz, 1H), 7.64 (t, J 7.7 Hz, 1H), 7.33 (d, J 8.8 Hz, 1H), 5.67 (s, 1H), 4.71 (d, J 6.6 Hz, 2H), 4.08 (q, J7.1 Hz, 2H), 3.97 (s, 3H), 3.70-3.57 (m, 4H), 3.43 (s, 4H), 3.26 (s, 3H), 3.21 (s, 3H), 2.55 (s, 3H), 1.21 (t, J 7.1 Hz, 3H). LCMS (ES+) [M+H]⁺ 657, RT 3.20 minutes (method 1).

### Example 21

### Ethyl 4-[3-[3-(difluoromethoxy)-4-methoxyphenyl]-7-({[3-(methylsulfonyl)phenyl]-methyl}amino)-2-methylpyrazolo[1,5-a]pyrimidin-5-yl]piperazine-1-carboxylate

Prepared from Intermediate 42 according to the method described for Example 1. The crude residue was purified by basic preparative HPLC. The relevant fractions were combined and concentrated in vacuo, then the residue was purified by SCX-2 (elution with MeOH followed by 7N ammonia in MeOH), to afford the title compound. δ_{H} (DMSO-d₆, 500 MHz) 8.25 (t, J 6.7 Hz, 1H), 8.08 (s, 1H), 7.83 (d, J 7.7 Hz, 1H), 7.81-7.77 (m, 2H), 7.63 (t, J 7.8 Hz, 1H), 7.52 (dd, J 8.6, 2.1 Hz, 1H), 7.25-6.90 (m, 2H), 5.65 (s, 1H), 4.70 (d, J 6.6 Hz, 2H), 4.07 (q, J 7.1 Hz, 2H), 3.84 (s, 3H), 3.66-3.55 (m, 4H), 3.42 (s, 4H), 3.21 (s, 3H), 2.51 (s, 3H, obscured by DMSO peaks), 1.20 (t, J 7.1 Hz, 3H). LCMS (ES+) [M+H]⁺ 645, RT 3.83 minutes (method 1).

### Example 22

### Ethyl 4-[3-(1,3-dimethyl-1H-indazol-5-yl)-7-({[3-(methanesulfonyl)phenyl]methyl}-amino)-2-methylpyrazolo[1,5-a]pyrimidin-5-yl]piperazine-1-carboxylate

Prepared from Intermediate 43 according to the method described for Example 2. δ_{H} (DMSO-d₆, 500 MHz) 8.23 (t, J 6.7 Hz, 1H), 8.10 (s, 1H), 7.95 (s, 1H), 7.84 (d, J 7.9 Hz, 1H), 7.82-7.76 (m, 2H), 7.64 (t, J 7.7 Hz, 1H), 7.56 (d, J 8.8 Hz, 1H), 5.66 (s, 1H), 4.71 (d, J 6.7 Hz, 2H), 4.06 (q, J7.1 Hz, 2H), 3.96 (s, 3H), 3.62-3.55 (m, 4H), 3.48-3.40 (m, 4H), 3.22 (s, 3H), 2.53 (s, 3H), 2.48 (s, 3H), 1.20 (t, J 7.1 Hz, 3H). LCMS (ES+) [M+H]⁺ 617, RT 3.44 minutes (method 1).

### Example 23

### 3-(3,4-Dimethoxyphenyl)-2-methyl-N-[(5-methylisoxazol-3-yl)methyl]-5-(morpholin-4-yl)pyrazolo[1,5-a]pyrimidin-7-amine

Intermediate 16 (0.41 g, 1.01 mmol) was dissolved in 1,2-dimethoxyethane (5 mL), then 3,4-dimethoxyphenylboronic acid (0.212 g, 1.1 mmol) and potassium phosphate tribasic hydrate (0.427 g, 2.01 mmol) were added. To the mixture were added water (1 mL) and [1,1′-bis(diphenylphosphino)ferrocene]dichloropalladium(II) complex with DCM (0.041 g, 0.050 mmol). The reaction mixture was stirred at 90° C. overnight, then concentrated in vacuo. The residue was partitioned between DCM and water. The organic phase was dried, filtered and concentrated in vacuo. The resulting brown oil was purified by column chromatography on silica (gradient elution with 0-5% MeOH/DCM). The resulting material was further purified by recrystallisation from MeOH to afford the title compound (40 mg, 9%) as a white solid. δ_{H} (400 MHz, DMSO-d₆) 7.92 (t, J 6.6 Hz, 1H), 7.55 (d, J 2.0 Hz, 1H), 7.18 (dd, J 8.4, 2.0 Hz, 1H), 6.98 (d J 8.4 Hz, 1H), 6.18 (s, 1H), 5.68 (s, 1H), 4.60 (d, J 6.5 Hz, 2H), 3.78 (s, 3H), 3.77 (s, 3H), 3.68 (t, J 4.8 Hz, 4H), 3.54 (t, J 4.8 Hz, 4H), 2.50 (s, 3H), 2.37 (s, 3H). LCMS (ES+) [M+H]⁺ 465.2, RT 2.16 minutes (method 9). LCMS (ES+) [M+H]⁻ 465.2, RT 2.19 minutes (method 3).

### Example 24

### Ethyl 4-[7-amino-3-(3,4-dimethoxyphenyl)-2-methylpyrazolo[1,5-a]pyrimidin-5-yl]-piperazine-1-carboxylate

To Intermediate 17 (120 mg, 0.25 mmol) and 3,4-dimethoxyphenylboronic acid (68 mg, 0.37 mmol) in a 20 mL pressure tube were added 1,4-dioxane (6 mL), 1M aqueous tripotassium phosphate solution (0.74 mL) and water (0.75 mL). The mixture was degassed with nitrogen for 3 minutes, then tetrakis(triphenylphosphine)palladium(0) (20 mg, 0.02 mmol) was added. The reaction mixture was sealed and heated at 90° C. with stirring for 2 h, then heated for a further 3.5 h at 100° C. To the resulting dark red mixture was added further 3,4-dimethoxyphenylboronic acid (68 mg, 0.37 mmol), and the reaction mixture was degassed with nitrogen for 3 minutes before tetrakis(triphenylphosphine)-palladium(0) (20 mg, 0.02 mmol) was added. The reaction mixture was heated at 120° C. with stirring for 3 h. The resulting black mixture was diluted with EtOAc (20 mL), then dried with anhydrous sodium sulfate and concentrated in vacuo. The resulting orange/brown oil was dissolved in DCM (4 mL), then TFA (1 mL, 13.07 mmol) was added. The reaction mixture was stirred at r.t. for 1.5 h, then quenched with saturated aqueous sodium hydrogen carbonate solution (20 mL) and extracted with DCM (2×20 mL). The organic layers were combined and dried with anhydrous sodium sulfate, then concentrated in vacuo. The residue was purified by column chromatography on silica (gradient elution with 0-4% MeOH in DCM), followed by basic preparative HPLC, followed by SCX column, to afford the title compound (32 mg, 29%) as a white solid. δ_{H} (DMSO-d₆, 500 MHz) 7.60 (d, J 2.0 Hz, 1H), 7.20-7.10 (m, 3H), 6.97 (d, J 8.4 Hz, 1H), 5.57 (s, 1H), 4.07 (q, J7.1 Hz, 2H), 3.79 (s, 3H), 3.76 (s, 3H), 3.59-3.53 (m, 4H), 3.51-3.43 (m, 4H), 2.48 (s, 3H), 1.20 (t, J 7.1 Hz, 3H). LCMS (ES+) [M+H]⁻ 441.2, RT 2.81 minutes (method 1).

### Example 25

### Ethyl 4-{7-amino-3-[4-chloro-3-(methylcarbamoyl)phenyl]-2-methylpyrazolo[1,5-a]-pyrimidin-5-yl}piperazine-1-carboxylate

To Intermediate 17 (120 mg, 0.25 mmol) and Intermediate 21 (80%, 138 mg, 0.37 mmol) in a 20 mL pressure tube were added 1,4-dioxane (6 mL), 1M aqueous tripotassium phosphate solution (0.74 mL) and water (0.75 mL). The mixture was degassed with nitrogen for 3 minutes, then tetrakis(triphenylphosphine)palladium(0) (20 mg, 0.02 mmol) was added. The reaction mixture was sealed and heated at 90° C. with stirring for 2 h, then heated for a further 3.5 h at 100° C. Further Intermediate 21 (80%, 138 mg, 0.37 mmol) was added, and the reaction mixture was degassed with nitrogen for 3 minutes before tetrakis(triphenylphosphine)palladium(0) (20 mg, 0.02 mmol) was added. The reaction mixture was heated to 120° C. with stirring for 3 h, then diluted with EtOAc (20 mL). The mixture was dried with anhydrous sodium sulfate and concentrated in vacuo. The resulting oil was dissolved in DCM (4 mL), then TFA (1 mL, 13.07 mmol) was added. The reaction mixture was stirred at r.t. for 1.5 h, then quenched with saturated aqueous sodium hydrogen carbonate solution (20 mL) and extracted with DCM (2×20 mL). The organic phases were combined, dried with anhydrous sodium sulfate and concentrated in vacuo. The resulting orange oil was purified by flash column chromatography on silica (gradient elution with 0-10% MeOH in DCM), followed by basic preparative HPLC, to afford the title compound (25 mg, 21%) as a white solid. δ_{H} (DMSO-d₆, 500 MHz) 8.35 (q, J4.4 Hz, 1H), 7.99 (d, J 2.2 Hz, 1H), 7.83 (dd, J 8.5, 2.3 Hz, 1H), 7.46 (d, J 8.5 Hz, 1H), 7.24 (s, 2H), 5.59 (s, 1H), 4.07 (q, J 7.1 Hz, 2H), 3.63-3.55 (m, 4H), 3.55-3.41 (m, 4H), 2.77 (d, J 4.6 Hz, 3H), 2.54-2.46 (m, 3H), 1.21 (t, J 7.1 Hz, 3H). LCMS (ES+) [M+H]⁻ 472.1, RT 2.67 minutes (method 1).

### Example 26

### 8-(5,6-Dimethoxypyridin-3-yl)-7-methyl-2-(morpholin-4-yl)pyrazolo[1,5-a][1,3,5]triazin-4-amine

To Intermediate 47 (200 mg, 0.56 mmol) and 2,3-dimethoxy-5-(4,4,5,5-tetramethyl-1,3,2-dioxaborolan-2-yl)pyridine (180 mg, 0.68 mmol) in 1,4-dioxane (4 mL) in a pressure tube was added 1M aqueous potassium carbonate solution (1.67 mL). The mixture was degassed with nitrogen for 3 minutes before [1,1′-bis(diphenylphosphino)-ferrocene]dichloropalladium(II) complex with DCM (30 mg, 0.037 mmol) was added. The reaction mixture was sealed and heated at 140° C. with stirring for 3 h. Further 2,3-dimethoxy-5-(4,4,5,5-tetramethyl-1,3,2-dioxaborolan-2-yl)pyridine (180 mg, 0.68 mmol) and 1M aqueous potassium carbonate solution (1.67 mL) were added. The reaction mixture was degassed for 3 minutes with nitrogen before [1,1′-bis(diphenylphosphino)-ferrocene]dichloropalladium(II) complex with DCM (30 mg, 0.04 mmol) was added. The reaction mixture was heated at 140° C. for 3 h with stirring, then allowed to cool. Water was added to the mixture. The resulting black solid (100 mg) was collected by filtration, then purified by flash column chromatography on silica (gradient elution with 0-100% EtOAc/heptane). To the resulting white solid (24 mg) was added DMSO, then the mixture was filtered and water was added to the filtrate. The white precipitate that formed was collected by filtration and was washed with water:DMSO (1:1), followed by water, to afford the title compound (12 mg, 5%) as a white solid. δ_{H} (DMSO-d₆, 500 MHz) 8.24-7.81 (m, 3H), 7.77 (d, J 1.9 Hz, 1H), 3.87 (s, 3H), 3.81 (s, 3H), 3.74-3.69 (m, 4H), 3.67-3.62 (m, 4H), 2.47 (s, 3H). LCMS (ES+) [M+H]⁺ 372.2, RT 2.57 minutes (method 1).

### Example 27

### 8-(3-Fluoro-5-methoxyphenyl)-7-methyl-2-(morpholin-4-yl)pyrazolo[1,5-a][1,3,5]triazin-4-amine

To Intermediate 47 (200 mg, 0.56 mmol) and 3-fluoro-5-methoxyphenylboronic acid (142 mg, 0.83 mmol) in 1,4-dioxane (4 mL) in a microwave tube was added 1M aqueous potassium carbonate solution (1.67 mL). The mixture was degassed with nitrogen for 3 minutes before [1,1′-bis(diphenylphosphino)ferrocene]dichloro-palladium(II) complex with DCM (30 mg, 0.04 mmol) was added. The reaction mixture was heated under microwave irradiation at 140° C. with stirring for 2 h, then allowed to cool and diluted with water (10 mL). The precipitate was collected by filtration, then purified by flash column chromatography on silica (gradient elution with 0-100% EtOAc/heptane). The resulting off-white solid was dissolved in EtOAc and filtered to remove insoluble material. The filtrate was concentrated. The resulting solid (70 mg) was further purified by acidic preparative HPLC to afford the title compound (20 mg, 10%) as a white solid. δ_{H} (DMSO-d₆, 500 MHz) 8.16 (s, 1H), 7.92 (s, 1H), 7.29-7.24 (m, 1H), 7.16 (dt, J 10.9, 2.0 Hz, 1H), 6.61 (dt, J11.0, 2.3 Hz, 1H), 3.79 (s, 3H), 3.76-3.69 (m, 4H), 3.69-3.61 (m, 4H), 2.56-2.41 (m, 3H). LCMS (ES+) [M+H]⁺ 359.1, RT 3.30 minutes (method 1).

### Example 28

### 8-(3,4-Dimethoxyphenyl)-2-(4-ethylpiperazin-1-yl)-7-methylpyrazolo[1,5-a][1,3,5]-triazin-4-amine

To a suspension of dimethyl N-cyanodithioiminocarbonate (4.2 g) in ethanol (100 mL) were added 4-(3,4-dimethoxyphenyl)-3-methyl-1H-pyrazol-5-amine (24.1 mmol) and piperidine (0.12 g, 1.4 mmol). The reaction mixture was heated to reflux for 24 h, then cooled to r.t. The precipitate was filtered and dried. To a suspension of the residue (0.3 mmol) in 1,4-dioxane (2 mL) was added 1-ethylpiperazine (0.42 mmol). The reaction mixture was heated at reflux for 24 h, then concentrated in vacuo. The residue was washed with water, then recrystallized from acetonitrile (3 mL) and ethanol (3 mL), to afford the title compound. LCMS (ES+) [M+H]⁺ 398.2, RT 0.83 minutes (method 4).

### Example 29

### Ethyl 4-[4-amino-8-(3,4-dimethoxyphenyl)-7-methylpyrazolo[1,5-a][1,3,5]triazin-2-yl]-piperazine-1-carboxylate

Prepared from 4-(3,4-dimethoxyphenyl)-3-methyl-1H-pyrazol-5-amine, dimethyl N-cyanodithioiminocarbonate and ethyl piperazine-1-carboxylate according to the method described for Example 28. δ_{H} (DMSO-d₆, 300 MHz) 8.04 (br m, 2H), 7.51 (d, J 2.0 Hz, 1H), 7.14 (dd, J 8.3, 2.0 Hz, 1H), 6.98 (d, J 8.4 Hz, 1H), 4.07 (q, J 7.1 Hz, 2H), 3.79 (s, 3H), 3.78-3.70 (m, 7H), 3.50-3.38 (m, 4H), 2.46 (s, 3H), 1.20 (t, J 7.1 Hz, 3H). LCMS (ES+) [M+H]⁺ 442.2, RT 0.89 minutes (method 4).

### Example 30

### 8-(3,4-Dimethoxyphenyl)-7-methyl-2-(morpholin-4-yl)pyrazolo[1,5-a][1,3,5]triazin-4-amine

Prepared from 4-(3,4-dimethoxyphenyl)-3-methyl-1H-pyrazol-5-amine, dimethyl N-cyanodithioiminocarbonate and morpholine according to the method described for Example 28. δ_{H} (DMSO-d₆, 300 MHz) 7.95 (s, 2H), 7.48 (d, J 2.0 Hz, 1H), 7.16 (dd, J 8.3, 2.0 Hz, 1H), 6.98 (d, J 8.4 Hz, 1H), 3.78 (s, 3H), 3.76 (s, 3H), 3.74-3.60 (m, 8H), 2.46 (s, 3H). LCMS (ES+) [M+H]^{|} 371.2, RT 0.80 minutes (method 4).

### Example 31

### 2-(Azepan-1-yl)-8-(3,4-dimethoxyphenyl)-7-methylpyrazolo[1,5-a][1,3,5]triazin-4-amine

Prepared from 4-(3,4-dimethoxyphenyl)-3-methyl-1H-pyrazol-5-amine, dimethyl N-cyanodithioiminocarbonate and hexamethyleneimine according to the method described for Example 28. LCMS (ES+) [M+H]⁺ 383.2, RT 1.02 minutes (method 4).

### Example 32

### 8-(3,4-Dimethoxyphenyl)-2-(4-isopropylpiperazin-1-yl)-7-methylpyrazolo[1,5-a][1,3,5]-triazin-4-amine

Prepared from 4-(3,4-dimethoxyphenyl)-3-methyl-1H-pyrazol-5-amine, dimethyl N-cyanodithioiminocarbonate and 1-isopropylpiperazine according to the method described for Example 28. LCMS (ES+) [M+H]⁺ 412.2, RT 0.87 minutes (method 4).

### Example 33

### 8-(3,4-Dimethoxyphenyl)-7-methyl-2-(4-methylpiperazin-1-yl)pyrazolo[1,5-a][1,3,5]-triazin-4-amine

Prepared from 4-(3,4-dimethoxyphenyl)-3-methyl-1H-pyrazol-5-amine, dimethyl N-cyanodithioiminocarbonate and 1-methylpiperazine according to the method described for Example 28. LCMS (ES+) [M+H]⁻ 384.2, RT 0.78 minutes (method 4).

### Example 34

### 8-(3,4-Dimethoxyphenyl)-7-methyl-2-[4-(pyridin-2-yl)piperazin-1-yl]pyrazolo[1,5-a]-[1,3,5]triazin-4-amine

Prepared from 4-(3,4-dimethoxyphenyl)-3-methyl-1H-pyrazol-5-amine, dimethyl N-cyanodithioiminocarbonate and 1-(pyridin-2-yl)piperazine according to the method described for Example 28. LCMS (ES+) [M+H]⁻ 447.2, RT 0.93 minutes (method 4).

### Example 35

### 8-(3,4-Dimethoxyphenyl)-7-methyl-2-(4-propylpiperazin-1-yl)pyrazolo[1,5-a][1,3,5 ]-triazin-4-amine

Prepared from 4-(3,4-dimethoxyphenyl)-3-methyl-1H-pyrazol-5-amine, dimethyl N-cyanodithioiminocarbonate and 1-propylpiperazine according to the method described for Example 28. LCMS (ES+) [M+H]⁻ 412.2, RT 0.91 minutes (method 4).

### Example 36

### 2-(4-Benzylpiperazin-1-yl)-8-(3,4-dimethoxyphenyl)-7-methylpyrazolo[1,5-a][1,3,5]-triazin-4-amine

Prepared from 4-(3,4-dimethoxyphenyl)-3-methyl-1H-pyrazol-5-amine, dimethyl N-cyanodithioiminocarbonate and 1-benzylpiperazine according to the method described for Example 28. LCMS (ES+) [M+H]⁻ 460.2, RT 1.01 minutes (method 4).

### Example 37

### 1-[4-(3-(3,4-Dimethoxyphenyl)-7-{[3-(N,S-dimethylsulfonimidoyl)phenyl]methyl-amino}-2-methylpyrazolo[1,5-a]pyrimidin-5-yl)piperazin-1-yl]ethanone

Intermediate 59 (105 mg, 0.18 mmol), 1-acetylpiperazine (34 mg, 0.27 mmol) and DIPEA (0.062 mL, 0.36 mmol) were heated at 110° C. in N,N-dimethylacetamide for 90 minutes, then at 160° C. overnight. Additional 1-acetylpiperazine (34 mg, 0.27 mmol) and DIPEA (0.062mL, 0.36 mmol) were added, and heating was continued for 8 h at 160° C. The reaction mixture was diluted with DCM (15 mL), then washed with saturated aqueous ammonium chloride solution (10 mL) and saturated aqueous sodium hydrogen carbonate solution (10 mL). The organic layer was dried over anhydrous sodium sulfate and solvent was removed in vacuo. The residue was purified by flash chromatography on silica (gradient elution with 0-10% MeOH/DCM). The resulting material (40 mg) was further purified by basic preparative HPLC to afford the title compound (15 mg, 14%) as a colourless powder. δ_{H} (DMSO-d₆, 300 MHz) 8.23 (t, J 6.7 Hz, 1H), 8.00-7.94 (m, 1H), 7.77-7.67 (m, 2H), 7.65-7.52 (m, 2H), 7.15 (dd, J 8.4, 2.0 Hz, 1H), 6.97 (d, J 8.5 Hz, 1H), 5.64 (s, 1H), 4.69 (d, J 6.6 Hz, 2H), 3.78 (s, 3H), 3.76 (s, 3H), 3.67-3.40 (m, 8H), 3.10 (s, 3H), 2.43 (s, 3H), 2.02 (s, 3H) (one methyl peak obscured by solvent peaks). LCMS (ES+) [M+H]⁺ 592, RT 1.88 minutes (method 3).

### Example 38

### 1-[4-(3-(3,4-Dimethoxyphenyl)-2-methyl-7-{[3-(methylsulfonimidoyl)phenyl]methyl-amino}pyrazolo[1,5-a]pyrimidin-5-yl)piperazin-1-yl]ethanone

Intermediate 61 (89 mg, 0.15 mmol) was dissolved in DCM (1.5 mL), then TFA (1.5 mL) was added. The reaction mixture was stirred at r.t. overnight, then concentrated in vacuo and purified by flash column chromatography on silica (gradient elution with 0-10% MeOH/DCM). The residue was further purified by preparative HPLC to afford the title compound (27 mg, 36%). δ_{H} (DMSO-d₆, 300 MHz) 8.22 (t, J 6.6 Hz, 1H), 8.11-8.05 (m, 1H), 7.82 (dt, J 7.8, 1.5 Hz, 1H), 7.74-7.67 (m, 1H), 7.61-7.52 (m, 2H), 7.15 (dd, J 8.4, 2.0 Hz, 1H), 6.97 (d, J 8.5 Hz, 1H), 5.64 (s, 1H), 4.68 (d, J 6.6 Hz, 2H), 4.23-4.17 (m, 1H), 3.78 (s, 3H), 3.76 (s, 3H), 3.67-3.40 (m, 8H), 3.05 (d, J 1.0 Hz, 3H), 2.02 (s, 3H) (one methyl peak obscured by solvent peaks). LCMS (ES+) [M+H]⁻ 578, RT 1.78 minutes (method 3).

### Example 39

### 1-(4-{3-(3,4-Dimethoxyphenyl)-2-methyl-7-[(2-methylpyridin-4-yl)methylamino]-pyrazolo[1,5-a]pyrimidin-5-yl}piperazin-1-yl)ethanone

Intermediate 65 was dissolved in 1:1 DCM/TFA (5 mL) and stirred at r.t. for 3 h. The reaction mixture was cooled and maintained at −20° C. for 3 days, then warmed to r.t. and concentrated in vacuo. The residue was dissolved in DCM (15 mL) and washed with saturated aqueous sodium carbonate solution, then dried over anhydrous sodium sulfate and concentrated in vacuo. The residue was purified by flash column chromatography on C18 silica [gradient elution with 0-100% acetonitrile (containing 0.1% ammonia)/10 mM aqueous ammonium hydrogen carbonate solution (containing 0.1% ammonia)] to afford the title compound (86 mg, 41.1%). δ_{H} (DMSO-d₆, 300 MHz) 8.37 (dd, J 5.2, 0.8 Hz, 1H), 8.12 (t, J 6.7 Hz, 1H), 7.57 (d, J 2.0 Hz, 1H), 7.25-7.22 (m, 1H), 7.20-7.12 (m, 2H), 6.98 (d, J 8.4 Hz, 1H), 5.55 (s, 1H), 4.59 (d, J 6.6 Hz, 2H), 3.79 (s, 3H), 3.76 (s, 3H), 3.64-3.43 (m, 8H), 2.43 (s, 3H), 2.02 (s, 3H) (one methyl signal obscured by solvent peak). LCMS (ES+) [M+H]^{|} 516, RT 1.85 minutes (method 3).

### Example 40

### 1-(4-{3-(1,3-Dimethylindazol-5-yl)-2-methyl-7-[(2-methylpyridin-4-yl)methylamino]-pyrazolo[1,5-a]pyrimidin-5-yl}piperazin-1-yl)ethanone

Prepared from Intermediate 66 according to the method described for Example 39 to afford the title compound (56.3 mg, 37.1%). δ_{H} (DMSO-d₆, 300 MHz) 8.38 (d, J 5.1 Hz, 1H), 8.14 (t, J 6.6 Hz, 1H), 7.97-7.93 (m, 1H), 7.80 (dd, J 8.8, 1.6 Hz, 1H), 7.59-7.53 (m, 1H), 7.25 (s, 1H), 7.22-7.15 (m, 1H), 5.57 (s, 1H), 4.61 (d, J 6.7 Hz, 2H), 3.96 (s, 3H), 3.54 (d, J 26.4 Hz, 8H), 2.53 (s, 3H), 2.44 (s, 3H), 2.02 (d, J 1.0 Hz, 3H) (one methyl signal obscured by solvent peak). LCMS (ES+) [M+H]⁺ 524, RT 1.90 minutes (method 3).

### Example 41

### 5-{5-(4-Acetylpiperazin-1-yl)-2-methyl-7-[(2-methylpyridin-4-yl)methylamino]pyrazolo-[1,5-a]pyrimidin-3-yl}-2-chloro-N-methylbenzamide

Prepared from Intermediate 67 according to the method described for Example 39 to afford the title compound (62.2 mg, 38.3%). δ_{H} (DMSO-d₆, 300 MHz) 8.40-8.33 (m, 2H), 8.21 (t, J 6.6 Hz, 1H), 7.94 (d, J 2.2 Hz, 1H), 7.83 (dd, J 8.5, 2.3 Hz, 1H), 7.47 (d, J 8.5 Hz, 1H), 7.24 (s, 1H), 7.21-7.14 (m, 1H), 5.58 (s, 1H), 4.59 (d, J 6.6 Hz, 2H), 3.66-3.41 (m, 8H), 2.77 (d, J 4.6 Hz, 3H), 2.53 (s, 3H), 2.43 (s, 3H), 2.03 (s, 3H). LCMS (ES+) [M+H]⁺ 547/549, RT 1.72 minutes (method 3).

### Example 42

### 1-[4-(3-(1,3-Dimethylindazol-6-yl)-2-methyl-7-{[3-(methylsulfonyl)phenyl]methyl-amino}pyrazolo[1,5-a]pyrimidin-5-yl)piperazin-1-yl]ethanone

Hydrogen chloride in 1,4-dioxane (4M, 2 mL) was added to a stirred solution of Intermediate 68 (129 mg, 0.088 mmol) in 1,4-dioxane (2 mL). The reaction mixture was stirred at ambient temperature for 1 h and concentrated in vacuo. Purification by flash column chromatography on C18 silica [gradient elution with 0-100% acetonitrile (containing 0.1% ammonia) in 10 mM aqueous ammonium hydrogen carbonate solution (containing 0.1% ammonia)] afforded the title compound (22 mg, 42%) as a pale yellow powder. δ_{H} (DMSO-d₆, 400 MHz) 8.07 (s, 1H), 7.89-7.76 (m, 4H), 7.69-7.62 (m, 2H), 7.55 (d, J 8.5 Hz, 1H), 5.63 (s, 1H), 4.76 (d, J 6.4 Hz, 2H), 3.95 (s, 3H), 3.70-3.60 (m, 4H), 3.59-3.47 (m, 4H), 3.18 (s, 3H), 2.58 (s, 3H), 2.03 (s, 3H) (one methyl signal obscured by solvent peak). LCMS (ES+) [M+H] 587, RT 2.11 minutes (method 3).

### Example 43

### 1-(4-{3-(3,4-Dimethoxyphenyl)-2-methyl-7-[(3-methyl-1,2,4-oxadiazol-5-yl)methyl-amino]pyrazolo[1,5-a]pyrimidin-5-yl}piperazin-1-yl)ethanone

Prepared from Intermediate 69, 1-acetylpiperazine and DIPEA according to the method described for Example 37 to afford the title compound (26 mg, 30.4%). δ_{H} (DMSO-d₆, 300 MHz) 8.01-7.91 (m, 1H), 7.58 (d, J 2.0 Hz, 1H), 7.18 (dd, J 8.3, 2.0 Hz, 1H), 6.99 (d, J 8.4 Hz, 1H), 5.84 (s, 1H), 4.95 (d, J 5.7 Hz, 2H), 3.80 (s, 3H), 3.77 (s, 3H), 3.71-3.47 (m, 8H), 2.32 (s, 3H), 2.04 (s, 3H) (one methyl signal obscured by solvent peak). LCMS (ES+) [M+H]⁺ 507.2, RT 1.88 minutes (method 3).

### Example 44

### Ethyl 4-[7-amino-3-(1,3-dimethyl-1H-indazol-5-yl)-2-methylpyrazolo[1,5-a]pyrimidin-5-yl]piperazine-1-carboxylate

Intermediate 73 (50 mg, 0.09 mmol) was dissolved in formic acid (2 mL), then 10% palladium on carbon (50% wet, 49 mg, 0.02 mmol) was added. The reaction mixture was heated at 70° C. with stirring for 1.5 h, then filtered through glass filter paper, washing with acetonitrile. The filtrate was concentrated in vacuo, and the residue was purified by preparative HPLC, to afford the title compound (11 mg, 26%) as a white solid. δ_{H} (DMSO-d₆, 500 MHz) 7.97 (s, 1H), 7.81 (dd, J 8.7, 1.4 Hz, 1H), 7.55 (d, J 8.8 Hz, 1H), 7.16 (s, 2H), 5.59 (s, 1H), 4.07 (q, J 7.1 Hz, 2H), 3.96 (s, 3H), 3.58-3.52 (m, 4H), 3.50-3.45 (m, 4H), 2.50 (s, 3H, methyl signal under DMSO peak observed in HSQC), 2.48 (s, 3H), 1.20 (t, J 7.1 Hz, 3H). LCMS (ES+) [M+H]⁻ 449.2, RT 2.80 minutes (method 1).

### Example 45

### 1-(4-{3-(3,4-Dimethoxyphenyl)-2-methyl-7-[(5-methyl-1,3,4-oxadiazol-2-yl)methyl-amino]pyrazolo[1,5-a]pyrimidin-5-yl}piperazin-1-yl)ethanone

Intermediate 74 (150 mg, 0.36 mmol), 1-acetylpiperazine (93 mg, 0.72 mmol) and DIPEA (143 mg, 1.09 mmol, 0.19 mL) were dissolved in N,N-dimethylacetamide (0.5 mL). The reaction mixture was heated at 140° C. for 3.5 h. The crude residue was purified by flash column chromatography on C18 silica [gradient elution with 0-100% acetonitrile in 10 mM aqueous ammonium bicarbonate solution (both spiked with 0.1% ammonia)] to afford the title compound (93 mg, 51%) as a beige solid. δ_{H} (DMSO-d₆, 400 MHz) 8.02 (t, J 6.4 Hz, 1H), 7.57 (d, J 2.0 Hz, 1H), 7.17 (dd, J 8.3, 2.0 Hz, 1H), 6.99 (d, J 8.4 Hz, 1H), 5.81 (s, 1H), 4.85 (d, J 6.4 Hz, 2H), 3.80 (s, 3H), 3.77 (s, 3H), 3.70-3.49 (m, 8H), 2.50 (s, 3H), 2.48 (s, 3H), 2.05 (s, 3H).

### Example 46

### 1-[4-(2-Methyl-7-{[3-(methylsulfonyl)phenyl]methylamino}-3-(3-methyl-[1,2,4]triazolo-[4,3-a]pyridin-6-yl)pyrazolo[1,5-a]pyrimidin-5-yl)piperazin-1-yl]ethanone

Hydrogen chloride in 1,4-dioxane (4M, 2 mL) was added to a stirred solution of Intermediate 75 (146 mg, 0.22 mmol) in 1,4-dioxane (2 mL). A solid immediately precipitated. MeOH (1 mL) was added, and the resulting solution was stirred for 1 h. The reaction mixture was concentrated in vacuo. The resulting brown foam was purified by preparative HPLC to afford the title compound (63 mg, 51%). δ_{H} (DMSO-d₆, 400 MHz) 8.61-8.55 (m, 1H), 8.33 (t, J 6.6 Hz, 1H), 8.11-8.02 (m, 1H), 7.87-7.76 (m, 3H), 7.73 (dd, J 9.6, 1.0 Hz, 1H), 7.63 (t, J 7.8 Hz, 1H), 5.69 (s, 1H), 4.72 (d, J 6.4 Hz, 2H), 3.67-3.46 (m, 8H), 3.21 (s, 3H), 2.70 (s, 3H), 2.58 (s, 3H), 2.03 (s, 3H). UPLC (ES+) [M+H]⁻ 574.4, RT 1.59 minutes (method 10).

### Example 47

### 1-[4-(2-Methyl-7-{[3-(methylsulfonyl)phenyl]methylamino}-3-(3-methyl-[1,2,4]triazolo-[4,3-a]pyridin-7-yl)pyrazolo[1,5-a]pyrimidin-5-yl)piperazin-1-yl]ethanone

A suspension of Intermediate 14 (400 mg, 0.64 mmol) and (3-methyl-[1,2,4]-triazolo[4,3-a]pyridin-7-yl)boronic acid (171 mg, 0.97 mmol) in 1,4-dioxane (10 mL) and 1M aqueous potassium phosphate tribasic solution (1.93 mL, 1.93 mmol) was purged using a stream of nitrogen for 10 minutes. Tetrakis(triphenylphosphine)palladium(0) (74 mg, 0.064 mmol) was added and the mixture was purged for a further 5 minutes. The reaction mixture was sealed and heated at 100° C. under microwave irradiation for 3 h, then at 110° C. for 3 h. The reaction mixture was diluted with DCM (20 mL) and washed with water (10 mL). The organic phase was dried over anhydrous sodium sulfate and concentrated in vacuo. The crude residue was purified by flash column chromatography on C18 silica (gradient elution with 0-100% acetonitrile in 10 mM ammonium bicarbonate solution, both containing 0.1% ammonia). The fractions containing product were combined and concentrated, before being taken up in 4M hydrogen chloride in 1,4-dioxane (4 mL). A solid immediately precipitated. MeOH (1 mL) was added and the resulting solution was stirred for 1 h. The reaction mixture was concentrated in vacuo. The resulting solid was purified by preparative HPLC to afford the title compound (20 mg, 5.5%) as a colourless powder. δ_{H} (DMSO-d₆, 300 MHz) 8.38 (t, J 6.7 Hz, 1H), 8.31 (dd, J 7.4, 1.0 Hz, 1H), 8.11-8.06 (m, 1H), 7.87-7.77 (m, 3H), 7.73 (dd, J 7.3, 1.6 Hz, 1H), 7.63 (t, J 7.7 Hz, 1H), 5.72 (s, 1H), 4.72 (d, J 6.6 Hz, 2H), 3.75-3.46 (m, 8H), 3.21 (s, 3H), 2.67 (s, 3H), 2.62 (s, 3H), 2.04 (s, 3H). LCMS (ES+) [M+H]⁺ 574, RT 1.58 minutes (method 3).

### Example 48

### 1-[4-(3-(1,3-Dimethylimidazo[1,5-a]pyridin-6-yl)-2-methyl-7-{[3-(methylsulfonyl)-phenyl]methylamino}pyrazolo[1,5-a]pyrimidin-5-yl)piperazin-1-yl]ethanone

To a mixture of Intermediate 14 (400 mg, 0.64 mmol) in 1,4-dioxane (10 mL) were added Intermediate 79 (491 mg, 1.33 mmol) and 1M aqueous potassium phosphate tribasic solution (1.9 mL, 1.9 mmol). The mixture was purged with nitrogen before tetrakis(triphenylphosphine)palladium(0) (74 mg, 0.064 mmol) was added. The mixture was heated at 95° C. for 5 h, then concentrated in vacuo and purified by flash column chromatography on silica (gradient elution with 0-100% EtOAc in isohexane, followed by 0-100% MeOH in EtOAc). The recovered yellow oil was dissolved in DCM (2 mL) and MeOH (0.5 mL). Hydrogen chloride in 1,4-dioxane (4M, 2 mL) was added. The reaction mixture was stirred for 2 h, then concentrated in vacuo. The residue was purified using preparative HPLC to afford the title compound (17 mg, 14%) as an off-white solid. δ_{H} (300 MHz, DMSO-d₆) 8.28 (t, J 6.8 Hz, 1H), 8.25-8.24 (m, 1H), 8.08 (t, J 1.8 Hz, 1H), 7.86-7.76 (m, 2H), 7.67-7.60 (m, 1H), 7.47 (dd, J 9.5, 1.1 Hz, 1H), 7.07 (dd, J 9.6, 1.3 Hz, 1H), 5.67 (s, 1H), 4.71 (d, J 6.6 Hz, 2H), 3.68-3.46 (m, 8H), 3.21 (s, 3H), 2.55 (s, 3H), 2.53 (s, 3H), 2.38 (s, 3H), 2.03 (s, 3H). LCMS (ES+) [M+H]⁻ 587, RT 1.94 minutes (method 3).

### Example 49

### 1-[4-(2-Methyl-3-(1-methylindazol-6-yl)-7-{[3-(methylsulfonyl)phenyl]methylamino}-pyrazolo[1,5-a]pyrimidin-5-yl)piperazin-1-yl]ethanone

To a solution of Intermediate 14 (400 mg, 0.64 mmol) in 1,4-dioxane (10 mL) were added 1-methyl-6-(4,4,5,5-tetramethyl-1,3,2-dioxaborolan-2-yl)indazole (166 mg, 0.64 mmol) and 1M aqueous potassium phosphate tribasic solution (1.9 mL, 1.9 mmol). The mixture was purged with nitrogen for 15 minutes prior to addition of tetrakis-(triphenylphosphine)palladium(0) (74.4 mg, 0.064 mmol). The mixture was heated at 95° C. for 1 h, then cooled to ambient temperature and concentrated in vacuo. The residue was dry-loaded onto silica for purification by flash column chromatography on silica (gradient elution with 0-100% EtOAc/isohexane, followed by 0-20% MeOH in EtOAc). The resulting yellow gum was dissolved in DCM (10 mL) and MeOH (1 mL), then treated with 4M hydrogen chloride in 1,4-dioxane (10 mL). After 90 minutes, the mixture was concentrated in vacuo. The residue was purified using reverse phase silica flash chromatography (pH 10, gradient elution with 0-100% acetonitrile in water) to afford the title compound (101 mg, 26%) as a white solid. δ_{H} (300 MHz, DMSO-d₆) 8.31-8.24 (m, 1H), 8.11-8.07 (m, 1H), 7.98-7.94 (m, 2H), 7.86-7.78 (m, 2H), 7.73 (dd, J 8.5, 0.8 Hz, 1H), 7.68-7.60 (m, 1H), 7.57 (dd, J 8.5, 1.3 Hz, 1H), 5.69 (s, 1H), 4.72 (d, J 6.6 Hz, 2H), 4.03 (s, 3H), 3.69-3.61 (m, 2H), 3.61-3.47 (m, 6H), 3.21 (s, 3H), 2.59 (s, 3H), 2.03 (s, 3H). LCMS (ES+) [M+H]⁺ 573 RT 2.01 minutes (method 3).

### Example 50

### 1-[4-(3-(2,1,3-Benzoxadiazol-5-yl)-2-methyl-7-{[3-(methylsulfonyl)phenyl]methyl-amino}pyrazolo[1,5-a]pyrimidin-5-yl)piperazin-1-yl]ethanone

To a solution of Intermediate 14 (400 mg, 0.64 mmol) in 1,4-dioxane (10 mL) were added 5-(4,4,5,5-tetramethyl-1,3,2-dioxaborolan-2-yl)benzo[c][1,2,5]oxadiazole (180 mg, 0.71 mmol) and 1M aqueous potassium phosphate tribasic solution (1.9 mL, 1.9 mmol). The solution was purged with nitrogen for 20 minutes prior to addition of tetrakis(triphenylphosphine)palladium(0) (74.4 mg, 0.064 mmol). The mixture was heated at 95° C. for 1 h, then cooled to ambient temperature and concentrated in vacuo. The residue was dry-loaded onto silica before being purified by flash column chromatography on silica (gradient elution with 0-100% EtOAc/isohexane, followed by 0-5% MeOH in EtOAc). The resulting yellow gum was dissolved in DCM (10 mL) and MeOH (1 mL), then treated with 4M hydrogen chloride in 1,4-dioxane (10 mL). After stirring for 90 minutes, the mixture was concentrated in vacuo. The residue was purified using reverse phase silica flash chromatography (pH 10, gradient elution with 0-100% acetonitrile/water) to afford the title compound (55 mg, 15%) as a bright yellow solid. δ_{H} (300 MHz, DMSO-d₆) 8.46-8.36 (m, 2H), 8.12-8.08 (m, 1H), 8.08-8.04 (m, 1H), 8.02 (dd, J 9.5, 1.0 Hz, 1H), 7.87-7.77 (m, 2H), 7.68-7.59 (m, 1H), 5.73 (s, 1H), 4.73 (d, J 6.5 Hz, 2H), 3.75-3.64 (m, 2H), 3.62-3.48 (m, 6H), 3.21 (s, 3H), 2.63 (s, 3H), 2.04 (s, 3H). LCMS (ES+) [M+H]⁺ 561, RT 2.28 minutes (method 3).

### Example 51

### 5-(5-(4-Acetylpiperazin-1-yl)-2-methyl-7-{[3-(methylsulfonyl)phenyl]methylamino}-pyrazolo[1,5-a]pyrimidin-3-yl)-1-methylindolin-2-one

To a solution of Intermediate 14 (240 mg, 0.39 mmol) in 1,4-dioxane (6 mL) were added 1M aqueous potassium phosphate tribasic solution (1.2 mL, 1.2 mmol) and 1-methyl-5-(4,4,5,5-tetramethyl-1,3,2-dioxaborolan-2-yl)indolin-2-one (116 mg, 0.425 mmol). The mixture was purged with nitrogen for 15 minutes before tetrakis(triphenyl-phosphine)palladium(0) (45 mg, 0.039 mmol) was added. The mixture was heated at 95° C. for 18 h, then cooled to ambient temperature and concentrated in vacuo. The residue was dry-loaded onto silica and purified using flash column chromatography on silica (gradient elution with 0-100% EtOAc/isohexane, followed by 0-10% MeOH/EtOAc). The resulting yellow foam was dissolved in DCM (5 mL) and MeOH (1 mL), then treated with 4M hydrogen chloride in 1,4-dioxane (5 mL). After 1 h, the mixture was concentrated in vacuo. The residue was purified using reverse phase silica flash chromatography (pH 10, gradient elution with 0-100% acetonitrile/water) to afford the title compound (77 mg, 34%) as a white solid. δ_{H} (300 MHz, DMSO-d₆) 8.22 (t, J 6.6 Hz, 1H), 8.11-8.06 (m, 1H), 7.86-7.77 (m, 2H), 7.66-7.59 (m, 3H), 7.00 (d, J 8.0 Hz, 1H), 5.64 (s, 1H), 4.70 (d, J 6.6 Hz, 2H), 3.63-3.44 (m, 10H), 3.21 (s, 3H), 3.14 (s, 3H), 2.48 (s, 3H), 2.02 (s, 3H). LCMS (ES+) [M+H]⁺ 588, RT 1.95 minutes (method 10).

### Example 52

### 5-(5-(4-Acetylpiperazin-1-yl)-2-methyl-7-{[3-(methylsulfonyl)phenyl]methylamino}-pyrazolo[1,5-a]pyrimidin-3-yl)-3H-isobenzofuran-1-one

To a solution of Intermediate 14 (110 mg, 0.140 mmol) in DMF (4 mL) were added 5-(4,4,5,5-tetramethyl-1,3,2-dioxaborolan-2-yl)isobenzofuran-1(3H)-one (53.0 mg, 0.195 mmol) and cesium carbonate (170 mg, 0.53 mmol). The mixture was purged with nitrogen for 10 minutes before tetrakis(triphenylphosphine)palladium(0) (20 mg, 0.018 mmol) was added. The mixture was heated at 95° C. for 90 minutes, then cooled to ambient temperature and concentrated in vacuo. The residue was dry-loaded onto silica and purified by flash column chromatography on silica (gradient elution with 0-100% EtOAc/isohexane, followed by 0-10% MeOH/EtOAc). The resulting yellow foam was dissolved in DCM (1 mL) and treated with TFA (1 mL). After 90 minutes, the mixture was concentrated in vacuo. The residue was purified using reverse phase silica flash chromatography (pH 10, gradient elution with 0-100% acetonitrile/water) to afford the title compound (37 mg, 47%) as a white solid. δ_{H} (300 MHz, DMSO-d₆) 8.35 (t, J 6.6 Hz, 1H), 8.13-8.03 (m, 3H), 7.86-7.76 (m, 3H), 7.63 (t, J 7.7 Hz, 1H), 5.71 (s, 1H), 5.43 (s, 2H), 4.72 (d, J 6.6 Hz, 2H), 3.72-3.45 (m, 8H), 3.21 (s, 3H), 2.59 (s, 3H), 2.03 (s, 3H). LCMS (ES+) [M+H]⁺ 575, RT 2.04 minutes (method 10).

### Example 53

### 4-(7-Amino-2-methylpyrazolo[1,5-a]pyrimidin-5-yl)-N-(4-methoxy-3-methylphenyl)-piperazine-1-carboxamide

To Intermediate 77 (86.5 mg, 0.26 mmol) dissolved in DCM (5 mL) was added 4M hydrogen chloride in 1,4-dioxane (1 mL). The reaction mixture was stirred under nitrogen at r.t. for 5 h, then concentrated in vacuo. To the resulting crude pale yellow solid (60.4 mg, 0.26 mmol) was added phenyl N-(4-methoxy-3-methylphenyl)carbamate (71.3 mg, 0.28 mmol), followed by acetonitrile (5 mL) and DIPEA (0.23 mL, 1.3 mmol). The reaction mixture was stirred at r.t. under nitrogen for approximately 24 h. A solid precipitate was formed which was filtered off under reduced pressure. The off-white solid was washed with acetonitrile/isohexane to afford the title compound (71 mg, 0.18 mmol) as a pale pink solid. δ_{H} (300 MHz, DMSO-d₆) 8.33 (s, 1H), 7.23-7.18 (m, 2H), 7.03 (s, 2H), 6.85-6.77 (m, 1H), 5.71 (s, 1H), 5.53 (s, 1H), 3.73 (s, 3H), 3.31 (s, 4H), 2.28 (s, 3H), 2.11 (s, 3H) (2×CH₂ signals under solvent peak). LCMS (ES+) [M+H]⁺ 396.2, RT 1.699 minutes (method 3).

### Example 54

### 5-{5-(4-Acetylpiperazin-1-yl)-2-methyl-7-[(3-methyl-1,2,4-oxadiazol-5-yl)methylamino]-pyrazolo[1,5-a]pyrimidin-3-yl}-2-chloro-N-methylbenzamide

To Intermediate 82 (295 mg, 0.54 mmol) dissolved in 1,4-dioxane (10 mL) were added potassium carbonate (166 mg, 1.19 mmol) and Intermediate 21 (189 mg, 0.64 mmol). The reaction mixture was purged with nitrogen for 5 minutes, then [1,1′-bis-(diphenylphosphino)ferrocene]dichloropalladium(II) complex with DCM (74 mg, 0.09 mmol) was added. The reaction mixture was purged with nitrogen for 2 minutes, then heated at 100° C. under nitrogen for 6 h and left to cool to r.t. overnight. The reaction mixture was filtered through a pad of celite, washing with DCM. The solvent was removed in vacuo. The resulting dark brown oil was purified by flash column chromatography on silica (gradient elution with 0-100% EtOAc/isohexane, followed by 0-20% MeOH/DCM). To the resulting material (155 mg, 0.24 mmol) dissolved in DCM (10 mL) was added 4M hydrogen chloride in 1,4-dioxane (2 mL). The reaction mixture was stirred under nitrogen at r.t. for 2 h, then concentrated in vacuo. The resulting brown solid was dissolved in 10% MeOH/DCM (25 mL) and washed with saturated aqueous sodium hydrogen carbonate solution (25 mL). The organic layer was separated and the aqueous phase was washed with further DCM (25 mL). The organic phases were combined and dried with anhydrous sodium sulfate, then filtered under reduced pressure. The solvent was removed in vacuo. The resulting brown solid was purified by preparative HPLC to afford the title compound (31 mg, 24%). δ_{H} (300 MHz, DMSO-d₆) 8.36 (q, J 4.5 Hz, 1H), 8.06 (t, J 6.5 Hz, 1H), 7.95 (d, J 2.3 Hz, 1H), 7.84 (dd, J 8.5 Hz, 2.3 Hz, 1H), 7.48 (d, J 8.4 Hz, 1H), 5.87 (s, 1H), 4.96 (d, J 6.6 Hz, 2H), 3.71-3.46 (m, 8H), 2.78 (d, J 4.7, 3H), 2.52 (s, 3H), 2.32 (s, 3H), 2.04 (s, 3H). UPLC (ES+) [M+H]⁺ 538.4, RT 1.886 minutes (method 10).

### Example 55

### 1-(4-{3-[3-(Difluoromethoxy)-4-methoxyphenyl]-2-methyl-7-[(3-methyl-1,2,4-oxadiazol-5-yl)methylamino]pyrazolo[1,5-a]pyrimidin-5-yl}piperazin-1-yl)ethanone

To Intermediate 82 (295 mg, 0.54 mmol) dissolved in 1,4-dioxane (10 mL) were added potassium carbonate (182 mg, 1.30 mmol) and 2-[3-(difluoromethoxy)-4-methoxy-phenyl]-4,4,5,5-tetramethyl-1,3,2-dioxaborolane (203 mg, 0.68 mmol). The reaction mixture was purged with nitrogen for 5 minutes prior to addition of [1,1′-bis(diphenyl-phosphino)ferrocene]dichloropalladium(II) complex with DCM (61 mg, 0.075 mmol). The reaction mixture was purged with nitrogen for 2 minutes, then heated at 100° C. under nitrogen for 9 h. The reaction mixture was filtered through a pad of celite, washing with DCM. The solvent was removed in vacuo. The resulting dark brown oil was purified by flash column chromatography on silica (gradient elution with 0-100% EtOAc/isohexane, followed by 0-20% MeOH/DCM). The crude material was dissolved in DCM (10 mL) and 4M hydrogen chloride in 1,4-dioxane (2 mL) was added. The reaction mixture was stirred under nitrogen at r.t. for 2 h, then concentrated in vacuo. The resulting brown solid was dissolved in 10% MeOH/DCM (25 mL) and washed with saturated aqueous sodium hydrogen carbonate solution (25 mL). The organic layer was separated and the aqueous phase was washed with further DCM (25 mL). The organic phases were combined and dried with anhydrous sodium sulfate, then filtered under reduced pressure. The solvent was removed in vacuo. The resulting brown solid was purified by preparative HPLC to afford the title compound (31 mg, 24%) as an off-white solid. δ_{H} (300 MHz, DMSO-d₆) 8.01 (t, J 6.6 Hz, 1H), 7.77 (d, J 2.2 Hz, 1H), 7.55 (dd, J 8.6, 2.2 Hz, 1H), 7.19 (d, J 8.7 Hz, 1H), 7.08 (t, J 74.7 Hz, 1H), 5.84 (s, 1H), 4.95 (d, J 6.5 Hz, 2H), 3.85 (s, 3H), 3.71-3.45 (m, 8H), 2.32 (s, 3H), 2.04 (s, 3H) (one methyl signal obscured under solvent peaks). UPLC (ES+) [M+H]⁺ 543.4, RT 2.402 minutes (method 10).

### Example 56

### 2-{3-(3,4-Dimethoxyphenyl)-7-[(2,4-dimethylthiazol-5-yl)methylamino]-2-methyl-pyrazolo[1,5-a]pyrimidin-5-yl}-1,3,4,7,8,8a-hexahydropyrrolo[1,2-a]pyrazin-6-one

Intermediate 78 (176 mg, 0.30 mmol) in 1,4-dioxane (5 mL) and water (1 mL) was treated with 3,4-dimethoxyphenylboronic acid (65 mg, 0.36 mmol), tetrakis-(triphenylphosphine)palladium(0) (35 mg, 0.029 mmol) and potassium phosphate tribasic (127 mg, 0.60 mmol). The reaction mixture was heated at 100° C. for 4 h, then cooled and left overnight. The reaction mixture was concentrated in vacuo, then partitioned between DCM and water. The material was purified by flash column chromatography on silica (gradient elution with 20-100% EtOAc/isohexane). The resulting yellow oil was taken up in 4M hydrogen chloride in 1,4-dioxane (5 mL), with the inclusion of DCM and MeOH to aid solution. The reaction mixture was stirred for 48 h, then concentrated in vacuo. The residue was partitioned between DCM and saturated aqueous sodium bicarbonate solution. The organic layer was concentrated in vacuo and purified by preparative HPLC to afford the title compound (32 mg, 20%) as a white solid. δ_{H} (300 MHz, DMSO-d₆) 7.94 (t, J 6.4 Hz, 1H), 7.62 (d, J 2.0 Hz, 1H), 7.13 (dd, J 8.4, 2.0 Hz, 1H), 6.98 (d, J 8.5 Hz, 1H), 5.74 (s, 1H), 4.69 (d, J 6.4 Hz, 2H), 4.66-4.57 (m, 1H), 4.46 (d, J 9.3 Hz, 1H), 3.86 (d, J 9.4 Hz, 1H), 3.80 (s, 3H), 3.77 (s, 3H), 3.66-3.50 (m, 1H), 2.91-2.59 (m, 3H), 2.48 (s, 3H), 2.38 (s, 3H), 2.34-2.23 (m, 2H), 2.22-2.05 (m, 1H), 1.69-1.52 (m, 1H) (one methyl signal obscured by solvent peak). LCMS (ES+) [M+H]⁺ 548, RT 2.03 minutes (method 3).

### Examples 57 to 89

### General Method

To the appropriate amine (2 eq.) were added Intermediate 84 (26 mg, 0.048 mmol, 1 eq.), acetonitrile (1.2 mL) and DIPEA (26 μL, 0.15 mmol, 3 eq.). The reaction mixture was heated at 100° C. under microwave irradiation for 2 h, then concentrated in vacuo. To the residue was added TFA (1 mL). The reaction mixture was stirred at 45° C. for up to 2 days, then concentrated in vacuo and purified by preparative HPLC, to yield the title compound. Retention time (RT), observed mass (M) and purity (%) were measured by Method 11.

| Ex. | Name | RT | M | Purity |
|---|---|---|---|---|
| 57 | 2-{4-[7-Amino-3-(3,4-dimethoxyphenyl)-2-methylpyrazolo[1,5-a] | 3.28 | 480.3 | 100 |
| | pyrimidin-5-yl]piperazin-1-yl}-1-(pyrrolidin-1-yl)ethanone | | | |
| 58 | 3-(3,4-Dimethoxyphenyl)-2-methyl-5-(4-methylpiperazin-1-yl)- | 3.11 | 383.2 | 100 |
| | pyrazolo[1,5-a]pyrimidin-7-amine | | | |
| 59 | 3-(3,4-Dimethoxyphenyl)-2-methyl-5-(4-methyl-1,4-diazepan-1- | 3.13 | 397.2 | 100 |
| | yl)pyrazolo[1,5-a]pyrimidin-7-amine | | | |
| 60 | 4-[7-Amino-3-(3,4-dimethoxyphenyl)-2-methylpyrazolo[1,5-a]- | 3.68 | 383.2 | 100 |
| | pyrimidin-5-yl]piperazin-2-one | | | |
| 61 | Ethyl N-{1-[7-amino-3-(3,4-dimethoxyphenyl)-2-methylpyrazolo- | 4.22 | 455.2 | 93.19 |
| | [1,5-a]pyrimidin-5-yl]piperidin-4-yl}carbamate | | | |
| 62 | 1-[7-Amino-3-(3,4-dimethoxyphenyl)-2-methylpyrazolo[1,5-a]- | 3.51 | 411.2 | 100 |
| | pyrimidin-5-yl]piperidine-3-carboxamide | | | |
| 63 | 1-{4-[7-Amino-3-(3,4-dimethoxyphenyl)-2-methylpyrazolo[1,5-a] | 3.88 | 425.2 | 100 |
| | pyrimidin-5-yl]-1,4-diazepan-1-yl}ethanone | | | |
| 64 | 2-[7-Amino-3-(3,4-dimethoxyphenyl)-2-methylpyrazolo[1,5-a]- | 3.94 | 423.2 | 100 |
| | pyrimidin-5-yl]-1,3,4,7,8,8a-hexahydropyrrolo[1,2-a]pyrazin-6-one | | | |
| 65 | 2-[7-Amino-3-(3,4-dimethoxyphenyl)-2-methylpyrazolo[1,5-a]- | 3.75 | 408.2 | 98.26 |
| | pyrimidin-5-yl]-1,3,3a,5,6,6a-hexahydrocyclopenta[c]pyrrol-4-one | | | |
| 66 | 3-(3,4-Dimethoxyphenyl)-5-{3-[(dimethylamino)methyl]azetidin- | 2.83 | 397.2 | 88.42 |
| | 1-yl}-2-methylpyrazolo[1,5-a]pyrimidin-7-amine | | | |
| 67 | 3-(3,4-Dimethoxyphenyl)-2-methyl-5-[4-(2-methylpyrazol-3-yl)- | 4.14 | 448.2 | 100 |
| | piperidin-1-yl]pyrazolo[1,5-a]pyrimidin-7-amine | | | |
| 68 | 3-(3,4-Dimethoxyphenyl)-2-methyl-5-[3-(1-methylimidazol-2-yl)- | 3.01 | 434.2 | 100 |
| | pyrrolidin-1-yl]pyrazolo[1,5-a]pyrimidin-7-amine | | | |
| 69 | 1-[7-Amino-3-(3,4-dimethoxyphenyl)-2-methylpyrazolo[1,5-a]- | 3.09 | 383.2 | 100 |
| | pyrimidin-5-yl]azetidine-3-carboxamide | | | |
| 70 | 1-[7-Amino-3-(3,4-dimethoxyphenyl)-2-methylpyrazolo[1,5-a]- | 3.33 | 411.2 | 100 |
| | pyrimidin-5-yl]-N,N-dimethylazetidine-3-carboxamide | | | |
| 71 | 3-(3,4-Dimethoxyphenyl)-2-methyl-5-(2-oxa-5-azabicyclo[2.2.1]- | 3.56 | 382.2 | 100 |
| | heptan-5-yl)pyrazolo[1,5-a]pyrimidin-7-amine | | | |
| 72 | 3-(3,4-Dimethoxyphenyl)-5-(6,6-dimethyl-3-azabicyclo[3.1.0]- | 4.40 | 394.2 | 97.48 |
| | hexan-3-yl)-2-methylpyrazolo[1,5-a]pyrimidin-7-amine | | | |
| 73 | 1-[7-Amino-3-(3,4-dimethoxyphenyl)-2-methylpyrazolo[1.5-a\|- | 3.42 | 411.2 | 100 |
| | pyrimidin-5-yl]piperidine-4-carboxamide | | | |
| 74 | rac-(1R,5S)-8-[7-Amino-3-(3,4-dimethoxyphenyl)-2-methyl- | 4.36 | 408.2 | 98 |
| | pyrazolo[1,5-a]pyrimidin-5-yl]-8-azabicyclo[3.2.1]octan-3-one | | | |
| 75 | 3-(3,4-Dimethoxyphenyl)-2-methyl-5-[4-(methylsulfonyl)- | 4.24 | 447.2 | 94.71 |
| | piperazin-1-yl]pyrazolo[1,5-a]pyrimidin-7-amine | | | |
| 76 | 3-(3,4-Dimethoxyphenyl)-5-[3-(methoxymethyl)azetidin-1-yl]-2- | 3.47 | 384.2 | 100 |
| | methylpyrazolo[1,5-a]pyrimidin-7-amine | | | |
| 77 | 1-[7-Amino-3-(3,4-dimethoxyphenyl)-2-methylpyrazolo[1,5-a]- | 3.74 | 453.3 | 98 |
| | pyrimidin-5-yl]-N,N-diethylpyrrolidine-3-carboxamide | | | |
| 78 | 3-(3,4-Dimethoxyphenyl)-5-(4-methoxypiperidin-1-yl)-2-methyl- | 4.18 | 398.2 | 98 |
| | pyrazolo[1,5-a]pyrimidin-7-amine | | | |
| 79 | Allyl N-{1-[7-amino-3-(3,4-dimethoxyphenyl)-2-methylpyrazolo- | 4.38 | 467.2 | 92.93 |
| | [1,5-a]pyrimidin-5-yl]piperidin-4-yl}carbamate | | | |
| 80 | 3-(3,4-Dimethoxyphenyl)-5-(3-methoxyazetidin-1-yl)-2-methyl- | 3.48 | 370.2 | 100 |
| | pyrazolo[1,5-a]pyrimidin-7-amine | | | |
| 81 | N-{1-[7-Amino-3-(3,4-dimethoxyphenyl)-2-methylpyrazolo[1,5-a] | 3.60 | 439.2 | 100 |
| | pyrimidin-5-yl]pyrrolidin-3-yl}-N-ethylacetamide | | | |
| 82 | 3-(3,4-Dimethoxyphenyl)-5-[3-(methoxymethyl)pyrrolidin-1-yl]- | 3.65 | 398.2 | 88.98 |
| | 2-methylpyrazolo[1,5-a]pyrimidin-7-amine | | | |
| 83 | 3-(3,4-Dimethoxyphenyl)-5-[3-(imidazol-1-yl)pyrrolidin-1-yl]-2- | 3.82 | 420.2 | 100 |
| | methylpyrazolo[1,5-a]pyrimidin-7-amine | | | |
| 84 | 3-(3,4-Dimethoxyphenyl)-2-methyl-5-[2-(1-methylpyrazol-4-yl)- | 4.12 | 450.2 | 100 |
| | morpholin-4-yl]pyrazolo[1,5-a]pyrimidin-7-amine | | | |
| 85 | 3-(3,4-Dimethoxyphenyl)-2-methyl-5-[2-(5-methyl-1,2,4- | 4.45 | 452.2 | 98.34 |
| | oxadiazol-3-yl)morpholin-4-yl]pyrazolo[1,5-a]pyrimidin-7-amine | | | |
| 86 | 3-(3,4-Dimethoxyphenyl)-5-[3-(1,1-dioxo-1,4-thiazinan-4-yl)- | 3.36 | 473.2 | 100 |
| | azetidin-1-yl]-2-methylpyrazolo[1,5-a]pyrimidin-7-amine | | | |
| 87 | 3-(3,4-Dimethoxyphenyl)-2-methyl-5-[3-(morpholin-4-yl)- | 3.07 | 425.2 | 100 |
| | azetidin-1-yl]pyrazolo[1,5-a]pyrimidin-7-amine | | | |
| 88 | 3-(3,4-Dimethoxyphenyl)-5-[4-(dimethylamino)piperidin-1-yl]-2- | 3.13 | 411.2 | 100 |
| | methylpyrazolo[1,5-a]pyrimidin-7-amine | | | |
| 89 | {4-[7-Amino-3-(3,4-dimethoxyphenyl)-2-methylpyrazolo[1,5-a]- | 4.00 | 482.3 | 100 |
| | pyrimidin-5-yl]piperazin-1-yl}(morpholin-4-yl)methanone | | | |

### Examples 90 to 122

### General Method

To the appropriate amine (2 eq.) were added Intermediate 86 (26 mg, 0.05 mmol, 1 eq.), acetonitrile (1.2 mL) and DIPEA (19 mg, 0.15 mmol, 3 eq.). The reaction mixture was heated at 100° C. under microwave irradiation for 2 h, then concentrated in vacuo. To the reaction mixture was added TFA (2 mL). The reaction mixture was stirred at r.t. overnight, then concentrated in vacuo and purified by preparative HPLC, to yield the title compound. Retention time (RT), observed mass (M) and purity (%) were measured by Method 11.

| Ex. | Name | RT | M | Purity |
|---|---|---|---|---|
| 90 | 2-(4-{3-(3,4-Dimethoxyphenyl)-2-methyl-7-[(2-methylpyridin-4- | 2.95 | 585.3 | 100 |
| | yl)methylamino]pyrazolo[1,5-a]pyrimidin-5-yl}piperazin-1-yl)-1- | | | |
| | (pyrrolidin-1-yl)ethanone | | | |
| 91 | 3-(3,4-Dimethoxyphenyl)-2-methyl-5-(4-methylpiperazin-1-yl)-N- | 2.80 | 488.3 | 100 |
| | [(2-methylpyridin-4-yl)methyl]pyrazolo[1,5-a]pyrimidin-7-amine | | | |
| 92 | 3-(3,4-Dimethoxyphenyl)-2-methyl-5-(4-methyl-1,4-diazepan-1- | 2.84 | 502.3 | 100 |
| | yl)-N-[(2-methylpyridin-4-yl)methyl]pyrazolo[1,5-a]pyrimidin-7- | | | |
| | amine | | | |
| 93 | 4-{3-(3,4-Dimethoxyphenyl)-2-methyl-7-[(2-methylpyridin-4-yl)- | 3.22 | 488.2 | 100 |
| | methylamino]pyrazolo[1,5-a]pyrimidin-5-yl}piperazin-2-one | | | |
| 94 | Ethyl N-(1-{3-(3,4-dimethoxyphenyl)-2-methyl-7-[(2-methyl- | 3.70 | 560.3 | 86.4 |
| | pyridin-4-yl)methylamino]pyrazolo[1,5-a]pyrimidin-5-yl}- | | | |
| | piperidin-4-yl)carbamate | | | |
| 95 | 1-{3-(3,4-Dimethoxyphenyl)-2-methyl-7-[(2-methylpyridin-4-yl)- | 3.26 | 516.3 | 100 |
| | methylamino]pyrazolo[1,5-a]pyrimidin-5-yl}piperidine-3- | | | |
| | carboxamide | | | |
| 96 | 1-(4-{3-(3,4-Dimethoxyphenyl)-2-methyl-7-[(2-methylpyridin-4- | 3.40 | 530.3 | 100 |
| | yl)methylamino]pyrazolo[1,5-a]pyrimidin-5-yl}-1,4-diazepan-1- | | | |
| | yl)ethanone | | | |
| 97 | 2-{3-(3,4-Dimethoxyphenyl)-2-methyl-7-[(2-methylpyridin-4-yl)- | 3.39 | 528.3 | 100 |
| | methylamino]pyrazolo[1,5-a]pyrimidin-5-yl}-1,3,4,7,8,8a- | | | |
| | hexahydropyrrolo[1,2-a]pyrazin-6-one | | | |
| 98 | 2-{3-(3,4-Dimethoxyphenyl)-2-methyl-7-[(2-methylpyridin-4-yl)- | 3.51 | 513.3 | 100 |
| | methylamino]pyrazolo[1,5-a]pyrimidin-5-yl}-1,3,3a,5,6,6a- | | | |
| | hexahydrocyclopenta[c]pyrrol-4-one | | | |
| 99 | 3-(3,4-Dimethoxyphenyl)-2-methyl-5-[4-(2-methylpyrazol-3-yl)- | 3.65 | 553.3 | 99 |
| | piperidin-1-yl]-N-[(2-methylpyridin-4-yl)methyl]pyrazolo[1,5-a]- | | | |
| | pyrimidin-7-amine | | | |
| 100 | 3-(3,4-Dimethoxyphenyl)-2-methyl-5-[3-(1-methylimidazol-2- | 2.84 | 539.3 | 99 |
| | yl)pyrrolidin-1-yl]-N-[(2-methylpyridin-4-yl)methyl]pyrazolo[1,5- | | | |
| | a]pyrimidin-7-amine | | | |
| 101 | 3-(3,4-Dimethoxyphenyl)-2-methyl-5-(4-methyl-2,3,4a,5,7,7a- | 2.85 | 530.3 | 99 |
| | hexahydropyrrolo[3,4-b][1,4]oxazin-6-yl)-N-[(2-methylpyridin-4- | | | |
| | yl)methyl]pyrazolo[1,5-a]pyrimidin-7-amine | | | |
| 102 | 1-{3-(3,4-Dimethoxyphenyl)-2-methyl-7-[(2-methylpyridin-4-yl)- | 2.92 | 488.2 | 99 |
| | methylamino]pyrazolo[1,5-a]pyrimidin-5-yl}azetidine-3- | | | |
| | carboxamide | | | |
| 103 | 1-{3-(3,4-Dimethoxyphenyl)-2-methyl-7-[(2-methylpyridin-4-yl)- | 3.15 | 516.3 | 99 |
| | methylamino]pyrazolo[1,5-a]pyrimidin-5-yl}-N,N-dimethyl- | | | |
| | azetidine-3-carboxamide | | | |
| 104 | 3-(3,4-Dimethoxyphenyl)-2-methyl-N-[(2-methylpyridin-4-yl)- | 3.33 | 487.2 | 99 |
| | methyl]-5-(2-oxa-5-azabicyclo[2.2.1]heptan-5-yl)pyrazolo[1,5-a]- | | | |
| | pyrimidin-7-amine | | | |
| 105 | 3-(3,4-Dimethoxyphenyl)-5-(6,6-dimethyl-3-azabicyclo[3.1.0]- | 4.07 | 499.3 | 93.91 |
| | hexan-3-yl)-2-methyl-N-[(2-methylpyridin-4-yl)methyl]pyrazolo- | | | |
| | [1,5-a]pyrimidin-7-amine | | | |
| 106 | 1-{3-(3,4-Dimethoxyphenyl)-2-methyl-7-[(2-methylpyridin-4-yl)- | 3.21 | 516.3 | 100 |
| | methylamino]pyrazolo[1,5-a]pyrimidin-5-yl}piperidine-4- | | | |
| | carboxamide | | | |
| 107 | rac-(1R,5S]-8-{3-(3,4-Dimethoxyphenyl-2-methyl-7-[(2-methyl- | 3.65 | 513.3 | 96.31 |
| | pyridin-4-yl)methylamino]pyrazolo[1,5-a]pyrimidin-5-yl}-8-aza- | | | |
| | bicyclo[3.2.1]octan-3-one | | | |
| 108 | 3-(3,4-Dimethoxyphenyl)-2-methyl-N-[(2-methylpyridin-4-yl)- | 3.56 | 552.2 | 99 |
| | methyl]-5-[4-(methylsulfonyl)piperazin-1-yl]pyrazolo[1,5-a]- | | | |
| | pyrimidin-7-amine | | | |
| 109 | 3-(3,4-Dimethoxyphenyl)-5-[3-(methoxymethyl)azetidin-1-yl]-2- | 3.26 | 489.3 | 97 |
| | methyl-N-[(2-methylpyridin-4-yl)methyl]pyrazolo[1,5-a]- | | | |
| | pyrimidin-7-amine | | | |
| 110 | 1-{3-(3,4-Dimethoxyphenyl)-2-methyl-7-[(2-methylpyridin-4-yl)- | 3.55 | 558.3 | 100 |
| | methylamino]pyrazolo[1,5-a]pyrimidin-5-yl}-N,N-diethyl- | | | |
| | pyrrolidine-3-carboxamide | | | |
| 111 | 3-(3,4-Dimethoxyphenyl)-5-(4-methoxypiperidin-1-yl)-2-methyl- | 3.65 | 503.3 | 100 |
| | N-[(2-methylpyridin-4-yl)methyl]pyrazolo[1,5-a]pyrimidin-7- | | | |
| | amine | | | |
| 112 | Allyl N-(1-{3-(3,4-dimethoxyphenyl)-2-methyl-7-[(2-methyl- | 3.83 | 572.3 | 100 |
| | pyridin-4-yl)methylamino]pyrazolo[1,5-a]pyrimidin-5-yl}- | | | |
| | piperidin-4-yl)carbamate | | | |
| 113 | 3-(3,4-Dimethoxyphenyl)-5-(3-methoxyazetidin-1-yl)-2-methyl- | 3.27 | 475.2 | 98 |
| | N-[(2-methylpyridin-4-yl)methyl]pyrazolo[1,5-a]pyrimidin-7- | | | |
| | amine | | | |
| 114 | N-(1-{3-(3,4-Dimethoxyphenyl)-2-methyl-7-[(2-methylpyridin-4- | 3.39 | 544.3 | 99 |
| | yl)methylamino]pyrazolo[1,5-a]pyrimidin-5-yl}pyrrolidin-3-yl)- | | | |
| | N-ethylacetamide | | | |
| 115 | 3-(3,4-Dimethoxyphenyl)-5-[3-(methoxymethyl)pyrrolidin-1-yl]- | 3.47 | 503.3 | 95.43 |
| | 2-methyl-N-[(2-methylpyridin-4-yl)methyl]pyrazolo[1,5-a]- | | | |
| | pyrimidin-7-amine | | | |
| 116 | 3-(3,4-Dimethoxyphenyl)-5-[3-(imidazol-1-yl)pyrrolidin-1-yl]-2- | 3.53 | 525.3 | 87.81 |
| | methyl-N-[(2-methylpyridin-4-yl)methyl]pyrazolo[1,5-a]- | | | |
| | pyrimidin-7-amine | | | |
| 117 | 3-(3,4-Dimethoxyphenyl)-2-methyl-5-[2-(1-methylpyrazol-4-yl)- | 3.51 | 555.3 | 99 |
| | morpholin-4-yl]-N-[(2-methylpyridin-4-yl)methyl]pyrazolo[1,5- | | | |
| | a]pyrimidin-7-amine | | | |
| 118 | 3-(3,4-Dimethoxyphenyl)-2-methyl-5-[2-(5-methyl-1,2,4- | 3.67 | 557.3 | 95 |
| | oxadiazol-3-yl)morpholin-4-yl]-N-[(2-methylpyridin-4-yl)- | | | |
| | methyl]pyrazolo[1,5-a]pyrimidin-7-amine | | | |
| 119 | 3-(3,4-Dimethoxyphenyl)-5-[3-(1,1-dioxo-1,4-thiazinan-4-yl)- | 3.18 | 578.3 | 100 |
| | azetidin-1-yl]-2-methyl-N-[(2-methylpyridin-4-yl)methyl]- | | | |
| | pyrazolo[1,5-a]pyrimidin-7-amine | | | |
| 120 | 3-(3,4-Dimethoxyphenyl)-2-methyl-N-[(2-methylpyridin-4-yl)- | 2.87 | 530.3 | 100 |
| | methyl]-5-[3-(morpholin-4-yl)azetidin-1-yl]pyrazolo[1,5-a]- | | | |
| | pyrimidin-7-amine | | | |
| 121 | 3-(3,4-Dimethoxyphenyl)-5-[4-(dimethylamino)piperidin-1-yl]-2- | 2.87 | 516.3 | 99 |
| | methyl-N-[(2-methylpyridin-4-yl)methyl]pyrazolo[1,5-a]- | | | |
| | pyrimidin-7-amine | | | |
| 122 | (4-{3-(3,4-Dimethoxyphenyl)-2-methyl-7-[(2-methylpyridin-4- | 3.47 | 587.3 | 99 |
| | yl)methylamino]pyrazolo[1,5-a]pyrimidin-5-yl}piperazin-1-yl)- | | | |
| | (morpholin-4-yl)methanone | | | |

1. A compound of formula (I) or an N-oxide thereof, or a pharmaceutically acceptable salt or solvate thereof: 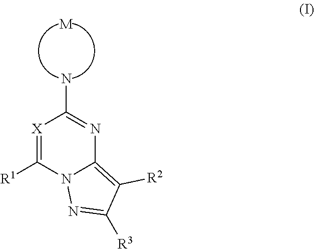
wherein
X represents N or CH;
M represents the residue of an optionally substituted saturated four-, five-, six- or seven-membered monocyclic ring containing one nitrogen atom and 0, 1, 2 or 3 additional heteroatoms independently selected from N, O and S, but containing no more than one O or S atom; or
M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom; or
M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom; or
M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom;
R¹ and R² independently represent hydrogen, halogen, cyano, nitro, hydroxy, trifluoromethyl, trifluoromethoxy, —OR^{a}, —SR^{a}, —SOR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b} or —SO₂NR^{b}R^{c}; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, heteroaryl or heteroaryl(C₁₋₆)alkyl, any of which groups may be optionally substituted by one or more substituents;
R³ represents hydrogen, halogen, cyano, trifluoromethyl or C₁₋₆ alkyl;
R^{a} represents hydrogen; or R^{a} represents C₁₋₆ alkyl, aryl, aryl(C₁₋₆)alkyl, heteroaryl or heteroaryl(C₁₋₆)alkyl, any of which groups may be optionally substituted by one or more substituents;
R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or
R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R^{d} represents hydrogen; or C₁₋₆ alkyl, C₃₋₇ cycloalkyl, aryl, C₃₋₇ heterocycloalkyl or heteroaryl, any of which groups may be optionally substituted by one or more sub stituents; and
R^{e} represents C₁₋₆ alkyl, aryl or heteroaryl, any of which groups may be optionally substituted by one or more substituents.

2. The compound as claimed in claim 1 wherein R¹ represents —NR^{b}R^{c}.

3. The compound as claimed in claim 1 represented by formula (IIA), or a pharmaceutically acceptable salt or solvate thereof:

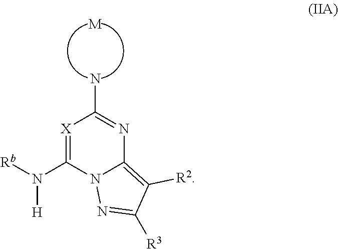

4. The compound as claimed in claim 1 wherein M represents the residue of an azetidin-1-yl, pyrrolidin-1-yl, piperidin-1-yl, morpholin-4-yl, piperazin-1-yl, azepan-1-yl or [1,4]diazepan-1-yl ring, or M represents the residue of a 1,2,3,3a,4,5,6,6a-octahydrocyclopenta[c]pyrrol-2-yl, 2,3,4,4a,5,6,7,7a-octahydropyrrolo-[3,4-b][1,4]oxazin-6-yl, 1,2,3,4,6,7,8,8a-octahydropyrrolo[1,2-a]-pyrazin-2-yl, 3-aza-bicyclo[3.1.0]hexan-3-yl, 2-oxa-5-azabicyclo[2.2.1]heptan-5-yl, 8-azabicyclo[3.2.1]-octan-8-yl or 2-oxa-6-azaspiro[3.3]heptan-6-yl ring system, any of which may be optionally substituted by one, two or three substituents independently selected from halogen, C₁₋₆ alkyl, benzyl, heteroaryl, (C₁₋₆)alkylheteroaryl, C₁₋₆ alkoxy, C₁₋₆ alkoxy-(C₁₋₆)alkyl, C₁₋₆ alkylsulfonyl, oxo, C₂₋₆ alkylcarbonyl, C₂₋₆ alkoxycarbonyl, di(C₁₋₆)alkyl-amino, di(C₁₋₆)alkylamino(C₁₋₆)alkyl, morpholinyl, dioxothiomorpholinyl, N—[(C₁₋₆)-alkyl]-N—[(C₂₋₆)alkylcarbonyl]amino, C₂₋₆ alkoxycarbonylamino, C₃₋₆ alkenyloxy-carbonylamino, aminocarbonyl, di(C₁₋₆)alkylaminocarbonyl, (C₁₋₆ alkoxy)(C₁₋₆ alkyl)-phenylaminocarbonyl, morpholinylcarbonyl and pyrrolidinylcarbonyl(C₁₋₆)alkyl.

5. The compound as claimed in claim 1 wherein R² represents hydrogen; or R² represents aryl, C₃₋₇ heterocycloalkyl or heteroaryl, any of which groups may be optionally substituted by one or two substituents independently selected from halogen, C₁₋₆ alkyl, C₁₋₆ alkoxy, difluoromethoxy, C₁₋₆ alkylsulfonyl, oxo and C₁₋₆ alkylaminocarbonyl.

6. The compound of formula (I) as defined in claim 1 as herein specifically disclosed in any one of the Examples.

7. (canceled)

8. (canceled)

9. A pharmaceutical composition comprising a compound of formula (I) as defined in claim 1 or an N-oxide thereof, or a pharmaceutically acceptable salt or solvate thereof, in association with a pharmaceutically acceptable carrier.

10. (canceled)

11. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 1 or an N-oxide thereof, or a pharmaceutically acceptable salt or solvate thereof.

12. The compound as claimed in claim 2 represented by formula (IIA), or a pharmaceutically acceptable salt or solvate thereof:

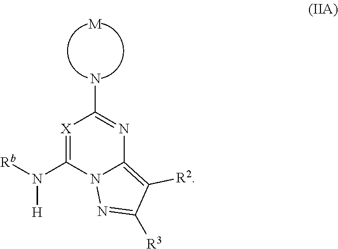

13. The compound as claimed in claim 2 wherein M represents the residue of an azetidin-1-yl, pyrrolidin-1-yl, piperidin-1-yl, morpholin-4-yl, piperazin-1-yl, azepan-1-yl or [1,4]diazepan-1-yl ring, or M represents the residue of a 1,2,3,3a,4,5,6,6a-octahydrocyclopenta[c]pyrrol-2-yl, 2,3,4,4a,5,6,7,7a-octahydropyrrolo-[3,4-b][1,4]oxazin-6-yl, 1,2,3,4,6,7,8,8a-octahydropyrrolo[1,2-a]-pyrazin-2-yl, 3-aza-bicyclo[3.1.0]hexan-3-yl, 2-oxa-5-azabicyclo[2.2.1]heptan-5-yl, 8-azabicyclo[3.2.1]-octan-8-yl or 2-oxa-6-azaspiro[3.3]heptan-6-yl ring system, any of which may be optionally substituted by one, two or three substituents independently selected from halogen, C₁₋₆ alkyl, benzyl, heteroaryl, (C₁₋₆)alkylheteroaryl, C₁₋₆ alkoxy, C₁₋₆ alkoxy-(C₁₋₆)alkyl, C₁₋₆ alkylsulfonyl, oxo, C₂₋₆ alkylcarbonyl, C₂₋₆ alkoxycarbonyl, di(C₁₋₆)alkyl-amino, di(C₁₋₆)alkylamino(C₁₋₆)alkyl, morpholinyl, dioxothiomorpholinyl, N—[(C₁₋₆)-alkyl]-N—[(C₂₋₆)alkylcarbonyl]amino, C₂₋₆ alkoxycarbonylamino, C₃₋₆ alkenyloxy-carbonylamino, aminocarbonyl, di(C₁₋₆)alkylaminocarbonyl, (C₁₋₆ alkoxy)(C₁₋₆ alkyl)-phenylaminocarbonyl, morpholinylcarbonyl and pyrrolidinylcarbonyl(C₁₋₆)alkyl.

14. The compound as claimed in claim 2 wherein R² represents hydrogen; or R² represents aryl, C₃₋₇ heterocycloalkyl or heteroaryl, any of which groups may be optionally substituted by one or two substituents independently selected from halogen, C₁₋₆ alkyl, C₁₋₆ alkoxy, difluoromethoxy, C₁₋₆ alkylsulfonyl, oxo and C₁₋₆ alkylaminocarbonyl.

15. The compound as claimed in claim 3 wherein M represents the residue of an azetidin-1-yl, pyrrolidin-1-yl, piperidin-1-yl, morpholin-4-yl, piperazin-1-yl, azepan-1-yl or [1,4]diazepan-1-yl ring, or M represents the residue of a 1,2,3,3a,4,5,6,6a-octahydrocyclopenta[c]pyrrol-2-yl, 2,3,4,4a,5,6,7,7a-octahydropyrrolo-[3,4-b][1,4]oxazin-6-yl, 1,2,3,4,6,7,8,8a-octahydropyrrolo[1,2-a]-pyrazin-2-yl, 3-aza-bicyclo[3.1.0]hexan-3-yl, 2-oxa-5-azabicyclo[2.2.1]heptan-5-yl, 8-azabicyclo[3.2.1]-octan-8-yl or 2-oxa-6-azaspiro[3.3]heptan-6-yl ring system, any of which may be optionally substituted by one, two or three substituents independently selected from halogen, C₁₋₆ alkyl, benzyl, heteroaryl, (C₁₋₆)alkylheteroaryl, C₁₋₆ alkoxy, C₁₋₆ alkoxy-(C₁₋₆)alkyl, C₁₋₆ alkylsulfonyl, oxo, C₂₋₆ alkylcarbonyl, C₂₋₆ alkoxycarbonyl, di(C₁₋₆)alkyl-amino, di(C₁₋₆)alkylamino(C₁₋₆)alkyl, morpholinyl, dioxothiomorpholinyl, N—[(C₁₋₆)-alkyl]-N—[(C₂₋₆)alkylcarbonyl]amino, C₂₋₆ alkoxycarbonylamino, C₃₋₆ alkenyloxy-carbonylamino, aminocarbonyl, di(C₁₋₆)alkylaminocarbonyl, (C₁₋₆ alkoxy)(C₁₋₆ alkyl)-phenylaminocarbonyl, morpholinylcarbonyl and pyrrolidinylcarbonyl(C₁₋₆)alkyl.

16. The compound as claimed in claim 3 wherein R² represents hydrogen; or R² represents aryl, C₃₋₇ heterocycloalkyl or heteroaryl, any of which groups may be optionally substituted by one or two substituents independently selected from halogen, C₁₋₆ alkyl, C₁₋₆ alkoxy, difluoromethoxy, C₁₋₆ alkylsulfonyl, oxo and C₁₋₆ alkylaminocarbonyl.

17. The compound as claimed in claim 15 wherein R² represents hydrogen; or R² represents aryl, C₃₋₇ heterocycloalkyl or heteroaryl, any of which groups may be optionally substituted by one or two substituents independently selected from halogen, C₁₋₆ alkyl, C₁₋₆ alkoxy, difluoromethoxy, C₁₋₆ alkylsulfonyl, oxo and C₁₋₆ alkylaminocarbonyl.

18. A pharmaceutical composition comprising a compound of formula (I) as defined in claim 2 or an N-oxide thereof, or a pharmaceutically acceptable salt or solvate thereof, in association with a pharmaceutically acceptable carrier.

19. A pharmaceutical composition comprising a compound of formula (I) as defined in claim 17 or an N-oxide thereof, or a pharmaceutically acceptable salt or solvate thereof, in association with a pharmaceutically acceptable carrier.

20. A pharmaceutical composition comprising a compound of formula (I) as defined in claim 6 or an N-oxide thereof, or a pharmaceutically acceptable salt or solvate thereof, in association with a pharmaceutically acceptable carrier.

21. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 3 or an N-oxide thereof, or a pharmaceutically acceptable salt or solvate thereof.

22. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 17 or an N-oxide thereof, or a pharmaceutically acceptable salt or solvate thereof.

23. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 6 or an N-oxide thereof, or a pharmaceutically acceptable salt or solvate thereof.
